(19) United States
(12) Patent Application Publication
(10) Pub. No.: US 2024/0228486 A1
(43) Pub. Date: Jul. 11, 2024

(54) SULFONE- AND SULFOXIMINE-BASED SELECTIVE PARP1 INHIBITORS

(71) Applicant: Enliven Inc., Boulder, CO (US)

(72) Inventors: Joseph P. LYSSIKATOS, Boulder, CO (US); Li REN, Superior, CO (US)

(21) Appl. No.: 18/545,788

(22) Filed: Dec. 19, 2023

Related U.S. Application Data
(60) Provisional application No. 63/433,905, filed on Dec. 20, 2022.

(51) Int. Cl. C07D 471/04; C07D 401/12; C07D 401/14

(52) U.S. Cl. CPC C07D 471/04; C07D 401/12; C07D 401/14

(57) ABSTRACT

Provided herein are compounds and compositions for selective inhibition of PARP1 over PARP2, methods of preparing said compounds and compositions, and their use in the treatment of various cancers, such as pancreatic, prostate, bladder, endometrial, lung, colorectal, ovarian, peritoneal, or breast cancer.

### CROSS-REFERENCE TO RELATED APPLICATION

This application claims priority to and benefit of U.S. Provisional Patent Application No. 63/433,905, filed on Dec. 20, 2022, the disclosure of which is hereby incorporated herein by reference in its entirety.

### FIELD

Provided herein are compounds and compositions for selective inhibition of PARP1 over PARP2, methods of preparing said compounds and compositions, and their use in the treatment of various cancers, such as pancreatic, prostate, bladder, endometrial, lung, colorectal, ovarian, peritoneal, or breast cancer.

### BACKGROUND

The covalent attachment of ADP-ribose to proteins (ADP-ribosylation) is a regulatory modality critically involved in modulating a cell's response to a variety of stresses including viral infection and DNA damage. ADP-ribosylation generally occurs on Glu, Asp or Lys residues of the target protein and comes in two forms depending upon whether a single ADP-ribose is attached to the target (mono-ADP ribosylation or MARylation) or multiple ADP-ribose moieties are appended to the target to form either branched or single-chain polymers (poly-ADP ribosylation or PARylation).

In higher eukaryotes, the enzymes that catalyze ADP-ribosylation are termed PARPs (poly-ADP-ribose polymerases). Based upon overall sequence homology, 17 members of the PARP family have been identified in humans. Of these, 1 is devoid of discernable catalytic activity (PARP13), 12 are mono-ADP ribose polymerases (PARP3, 4, 6-12 and 14-16) and 4 are poly-ADP ribose polymerases (PARP1, 2, Tankyrase 1, 2). In terms of total cellular PARylation activity, PARP1 and PARP2 are responsible for the vast majority of PARylation in cells with the former reportedly contributing more than 90%.

PARP1 is the predominant isoform involved in most of the cellular responses to DNA damage (DDR), but to varying degree dependent upon the type of damage and repair process involved. PARP1 knockout mice revealed that while viable and phenotypically normal, these animals exhibited increased genomic instability and enhanced sensitivity to treatments that induce DNA damage (e.g., ionizing radiation or exposure to alkylating agents). PARP2 is highly homologous to PARP1 and partially redundant with PARP1 function in the activation of the DNA damage response. Accordingly, the engineered deletion of both genes is early embryonic lethal in mice.

PARP1 binds to most types of DNA lesions and becomes activated to PARylate itself (auto-PARylation), creating ADP-ribose polymers that act as scaffolds for binding/recruitment of DNA repair enzymes. These repair enzymes are involved in the resolution of single-stranded lesions including breaks, nicks, base, or nucleotide modifications (via base-excision repair (BER), nucleotide excision repair (NER) or mismatch repair (MMR)) and double-stranded breaks (via either homologous recombination (HR) or the more common non-homologous end-joining (NHEJ)), as well as stalled replication forks during normal DNA synthesis. Whereas PARP1 is critical for NHEJ, NER and MMR, it is believed to play an important but non-essential role for both HR and BER as well as the surveillance of normal DNA replication. Once recruitment is complete, the PARylated PARP1 enzyme falls off the DNA lesion to allow for the repair to progress.

Genetic alterations in the genes coding for factors involved in the DDR that result in loss of function or expression are common in cancer. Accordingly, cancers harboring such alterations are defective for certain repair pathways and are therefore rendered dependent upon the remaining, semi-redundant pathways to resolve particular types of DNA damage. This new dependency creates what is known as a synthetic lethal condition in which the targeting of the remaining intact pathway will substantially reduce the cancer cell's ability to repair the DNA leading to widespread catastrophic DNA damage and ultimately cell death while sparing normal wildtype cells with a fully competent DDR. Consequently, tumors with mutations that impair HR or BER (the two major repair pathways not fully dependent upon PARP1) or that induce replication stress, have the potential to create an enhanced dependency upon PARP1 activity to survive. As an example, germline damaging mutations in BRCA1 or BRCA2, key mediators of HR, are highly correlated with the development of breast, bladder, colorectal, endometrial, lung, ovarian, pancreatic and prostate cancers. Loss of function in either of these factors profoundly eliminates the HR repair pathway in these tumors. As such, it has now been demonstrated both preclinically and clinically that small-molecule mediated inhibition of PARP1 activity elicits significant anti-tumor activity in cancers harboring either BRCA1 or 2 loss-of-function mutations. Beyond BRCA1/2, other commonly mutated DDR-associated factors involved in HR have been shown to be synthetically lethal with PARP1 inhibition. Importantly, other recurrent cancer-associated genetic alterations in other DDR repair pathways including double-stranded break repair (i.e., NBS1, MRE11) and BER (i.e., OGG1, Polq) have been shown to synergize with PARP inhibitors setting up additional therapeutic opportunities for this mechanism of action.

Given the promise of targeting PARP1 for the treatment of certain tumor types defective in one or more elements of the DDR, several small molecule PARP inhibitors have been clinically evaluated and most were ultimately approved for the treatment of ovarian, fallopian, peritoneal, breast and prostate cancers with HR deficiencies (e.g., BRCA1/2 mutations). These inhibitors include veliparib, olaparib, talazoparib, niraparib and rucaparib. These first-generation inhibitors have been shown to be essentially equipotent on PARP1 and PARP2 and to exhibit varying degrees of selectivity vs the other PARP family members. In addition, beyond simply inhibiting PARP activity, these agents have been shown to possess the added ability to lock PARP1 onto DNA lesions that then sterically hinders the ability of the DDR-associated factors to access the damaged site and repair the DNA.

Employing PARP inhibitors in the context of combinations with chemotherapies that elicit DNA damage may represent an important therapeutic opportunity to address a broader range of cancers that harbor an intact DDR. For example, therapies that induce double-stranded DNA breaks (e.g., topoisomerase I and II inhibitors) or DNA adducts (e.g., platinum-based drugs or alkylating agents) or interfere with DNA replication (e.g., anti-metabolites, nucleoside analogs) have been shown to effectively combine with various PARP1 inhibitors in preclinical studies and many of these combinations are currently undergoing clinical evaluation for the treatment of a variety of tumor types irrespective of mutational status. In addition, PARP inhibitor combinations with small molecule inhibitors that regulate the various repair pathways of the DDR (e.g., ATR, CHK1, WEE1) or targeted therapies (e.g., EGFR, MEK or VEGFR) are also being evaluated.

The clinical deployment of PARP inhibitors, whether as single agents or in combination, is limited by certain on-target toxicities. The predominant, likely on-target, dose-limiting (≥grade 3) toxicities are associated with the hematologic compartment, particularly anemia, neutropenia and to a lesser extent thrombocytopenia. Importantly, these same toxicities are associated with many of the combination therapies either being considered or currently undergoing clinical evaluation, most notably chemotherapeutic agents that induce DNA damage or replication stress or therapies that target the regulation of the DDR such as ATR, ATM, WEE1 or CHK1.

However, many of the clinical trials evaluating these combinations have reported significantly enhanced toxicities, particularly those related to myelosuppression. Therefore, next generation PARP inhibitors with an improved hematologic safety profile would represent an important advance for the field offering the promise of greater efficacy with a reduced risk of additive or synergistic toxicity in combination with other DDR-targeting agents. One approach currently being evaluated are PARP inhibitors with greater selectivity for PARP1 over PARP2. As mentioned above, all the approved PARP1 inhibitors possess limited or no selectivity vs PARP2. Because PARP2 has been specifically and exclusively linked to the maintenance of hematopoiesis particularly regarding the myelo-erythroid compartment, it is believed that inhibition of PARP2 is likely to be the driver of the anemia and potentially the other hematologic toxicities. Accordingly, efforts are underway to discover and develop PARP1-selective inhibitors for a better safety profile and improved combinability with standard of care chemotherapeutic agents. In fact, early pre-clinical and clinical data have reported reduced hematotoxicity with the PARP1-selective agent AZD-5305.

Thus, there is a need for improved PARP inhibitors and, more specifically, selective PARP1 inhibitors.

### BRIEF SUMMARY

In one aspect, provided herein is a compound of formula (I)

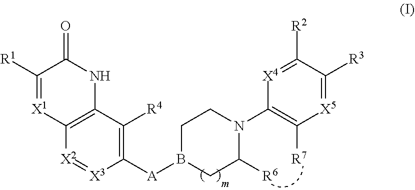

or a pharmaceutically acceptable salt, solvate, hydrate, or co-crystal thereof, or a mixture of any of the foregoing, wherein:
   X¹, X² and X³ are each independently N or C—R¹;
   X⁴ and X⁵ are each independently N or CH;
   A is —S(═O)₂—, —S(═O)(═NH)— or —S(═O)(═N—CH₃)—;
   B is N or CH;
   R¹ is H, halogen, C₁-C₄ alkyl, C₁-C₄ fluoroalkyl, C₁-C₄ alkoxy, or C₃-C₆ cycloalkyl;
   R² is H, halogen, C₁-C₄ alkyl, or C₁-C₄ fluoroalkyl;
   R³ is halogen or —C(═O)NH—R⁵;
   R⁴ is H, halogen, C₁-C₄ alkyl, or C₁-C₄ fluoroalkyl; and
   each R⁵ is independently H, CD₃, C₁-C₄ alkyl, C₁-C₄ fluoroalkyl, C₁-C₄ alkoxy, C₃-C₆ cycloalkyl, or 4- to 7-membered heterocycloalkyl;
   R⁶ is H; and
   R⁷ is H, halogen, CN, C₁-C₄ alkyl, C₁-C₄ fluoroalkyl, C₁-C₄ alkoxy, or C₃-C₆ cycloalkyl; or
   R⁶ and R⁷ are taken together with the atoms to which they are attached to form a ring, wherein R⁶ is —(CH₂)ₙ— and R⁷ is E, wherein E is —O—, —S—, —NR⁵—, or —CR⁵—;
   m is an integer of 1 or 2, wherein when R⁶ is H, then m is 1; and
   n is an integer of 1 or 2.

In some embodiments, the compound is a compound of formula (I-1)

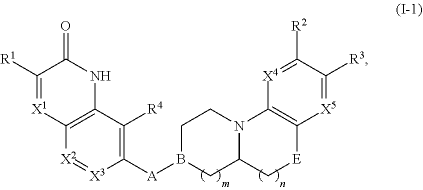

wherein:
E is —O—, —S—, —NR⁵—, or —CR⁵—;
m is an integer of 1 or 2; and
n is an integer of 1 or 2.

In some embodiments, the compound is a compound of formula (I-2)

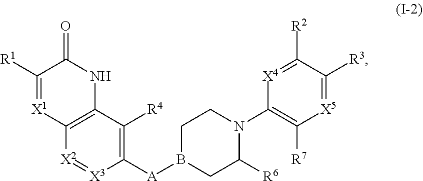

wherein:
R⁶ is H; and
R⁷ is H, halogen, CN, C₁-C₄ alkyl, C₁-C₄ fluoroalkyl, C₁-C₄ alkoxy, or C₃-C₆ cycloalkyl.

In some embodiments, A is —S(O)₂—. In some embodiments, A is S(O)(═NH)—. In some embodiments, A is —S(O)(═N—CH₃)—. In some embodiments, B is N. In some embodiments, B is CH.

In some embodiments, the compound the compound is a compound of formula (I-1-a), (I-1-b), (I-1-c), (I-1-d), (I-1-e), or (I-1-f):

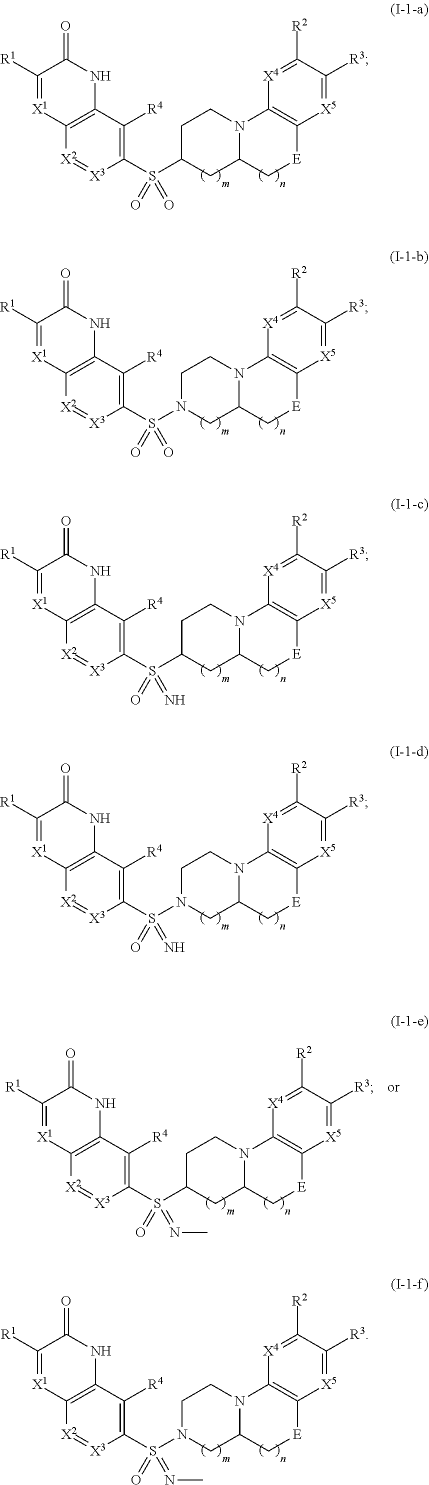

In some embodiments, the compound is a compound of formula (I-2-a), (I-2-b), (I-2-c), (I-2-d), (I-2-e), or (I-2-f):

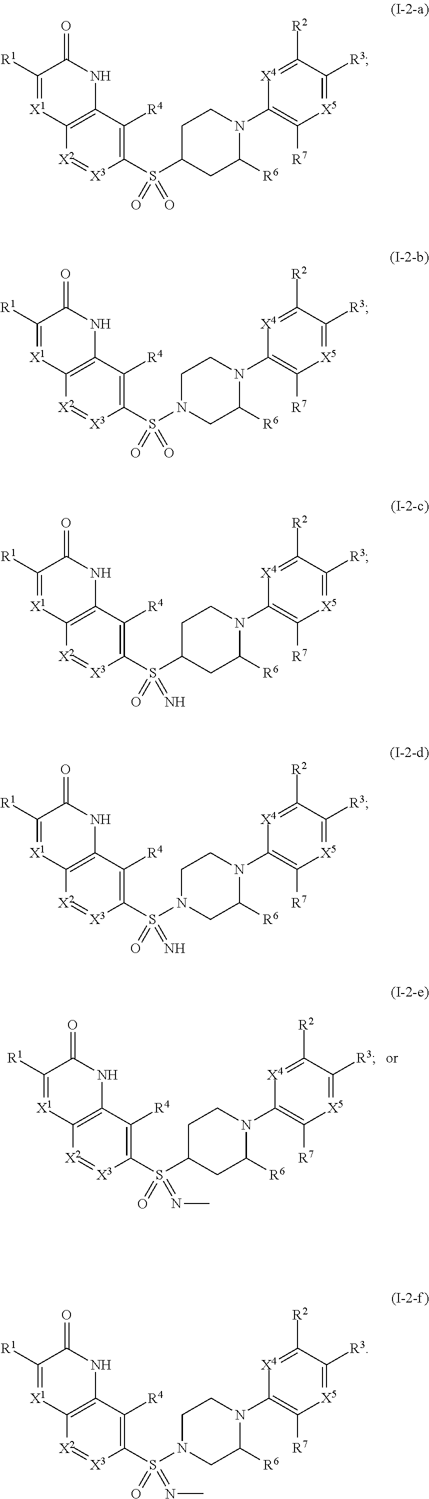

In some embodiments, X¹ is N. In some embodiments, X¹ is CH. In some embodiments, X² is N. In some embodiments, X² is CH. In some embodiments, one of X¹ and X² is N and the other of X¹ and X² is CH. In some embodiments, X³ is N. In some embodiments, X³ is CH or CF. In some embodiments, X⁴ is N. In some embodiments, X⁴ is CH. In some embodiments, X⁵ is N. In some embodiments, X⁵ is CH. In some embodiments, one of X¹ and X² is N and the other of X¹ and X² is CH; X³ is CH or CF; X⁴ is CH; and X⁵ is N or CH.

In some embodiments, R¹ is H or C₁-C₄ alkyl. In some embodiments, R¹ is H, —CH₃ or —CH₂CH₃. In some embodiments, R² is H or halogen. In some embodiments, R² is C₁-C₄ alkyl or C₁-C₄ fluoroalkyl. In some embodiments, R³ is halogen. In some embodiments, R³ is F or Cl. In some embodiments, R³ is —C(═O)NH—R⁵. In some embodiments, R⁵ is C₁-C₄ alkyl. In some embodiments, R⁵ is —CH₃. In some embodiments, R⁴ is H or halogen. In some embodiments, R⁴ is C₁-C₄ alkyl or C₁-C₄ fluoroalkyl. In some embodiments, R⁶ is H and R⁷ is H. In some embodiments, R⁶ is H and R⁷ is halogen. In some embodiments, R⁷ is F.

In another aspect, provided herein is a compound is selected from the group consisting of:

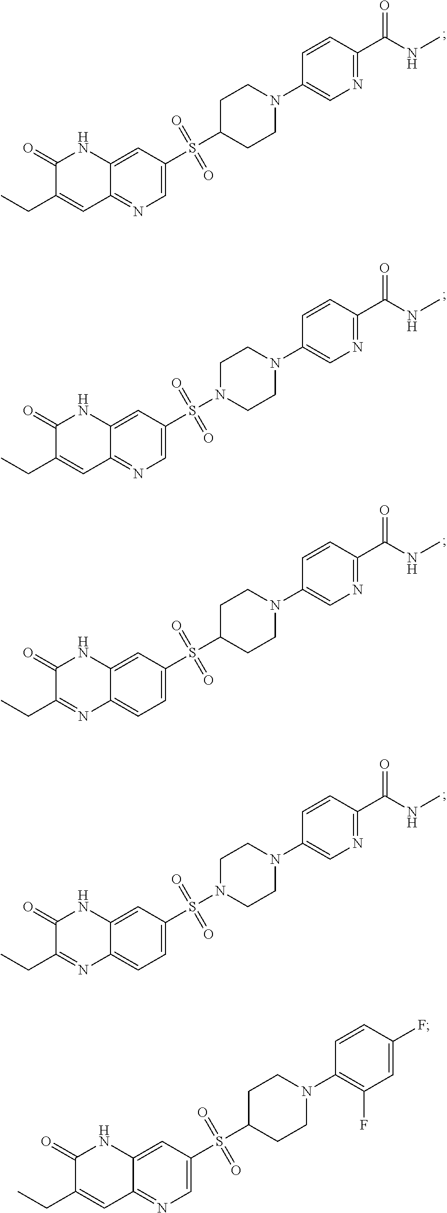

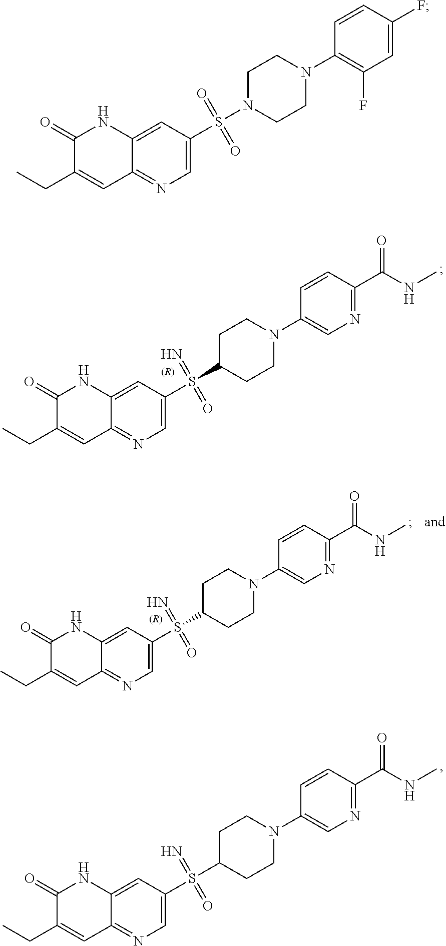

or a pharmaceutically acceptable salt, solvate, hydrate, or co-crystal thereof, or a mixture of any of the foregoing.

In some embodiments, provided herein is a pharmaceutical composition comprising a compound as described herein, or a pharmaceutically acceptable salt, solvate, hydrate, or co-crystal thereof, or a mixture of any of the foregoing, and one or more pharmaceutically acceptable excipients.

In another aspect, provided herein is a method of inhibiting PARP1 enzymatic activity in a cell, comprising exposing the cell with an effective amount of a compound according to the present disclosure, or a pharmaceutically acceptable salt, solvate, hydrate, or co-crystal thereof, or a mixture of any of the foregoing, or a pharmaceutical composition according to the present disclosure. In some embodiments, the cell is a cancer cell.

In another aspect, provided herein is a method of treating a cancer or neoplastic disease in a human in need thereof, comprising administering to the human a compound according to the present disclosure, or a pharmaceutically acceptable salt, solvate, hydrate, or co-crystal thereof, or a mixture of any of the foregoing, or a pharmaceutical composition according to the present disclosure.

In some embodiments, the cancer or neoplastic disease is associated with one or more mutations in the DNA damage response activation. In some embodiments, the one or more mutations in the DNA damage response activation is a mutation of ATM, ATR, BAP1, BARD1, BLM, BRCA1, BRCA2, BRIP1, CDK12, CHEK1, CHEK2, CCNE1 amplification, MRE11A, NBN, PALB2, PP2R2ARAD50, RAD51, RAD51B, RAD51C, RAD51D, RAD54L, XRCC2 and the FANC family of proteins. In some embodiments, the cancer or neoplastic disease is associated with one or more mutations in a DNA damage repair enzyme. In some embodiments, the repair enzyme assists in the repair of single-stranded lesions including breaks, nicks, base, or nucleotide modifications (via base-excision repair (BER), nucleotide excision repair (NER) or mismatch repair (MMR)) and double-stranded breaks (via either homologous recombination (HR) or the more common non-homologous end-joining (NHEJ)). In some embodiments, the cancer or neoplastic disease is pancreatic, prostate, bladder, endometrial, lung, colorectal, ovarian, peritoneal, or breast cancer. In some embodiments, the method further comprises administering one or more pharmaceutical agents that induces DNA damage and/or replicative stress, inhibits a protein or enzyme that regulates DDR or DNA replication, or induces anti-tumor immune response, or any combination(s) thereof. In some embodiments, the one or more pharmaceutical agents that induces DNA damage and/or replicative stress is an alkylating agent (e.g., temozolimide, nitrosoureas, mitomycin C), platinum-based anti-neoplastic agent (e.g., cisplatin, oxaliplatin, carboplatin), anti-metabolite (e.g., 5-fluorouracil (5-FU), gemcitabine, fludarabine, methotrexate), topoisomerase inhibitor (e.g., etoposide, irinotecan), ionizing radiation, radiomimetic, or other radiotherapy. In some embodiments, the one or more pharmaceutical agents that inhibits a protein or enzyme that regulates DDR or DNA replication is an inhibitor of ATR, ATM, DNA-PK, CHK1, CHK2, WEE1, MYT1, polθ, EGFR, ErbB2, MET, FGFR1-4, VEGFR2, MEK, ERK, PI3K, AKT, mTORC1, or mTORC2. In some embodiments, the one or more pharmaceutical agents induces anti-tumor immune response is anti-PD1 therapy, anti-PD-L1 therapy, anti-CTLA4 therapy, cancer vaccine, T-cell-based therapy, T-cell engager bi-specific antibody, or oncolytic virus.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### I. Definitions

As used herein, the following definitions shall apply unless otherwise indicated. Further, if any term or symbol used herein is not defined as set forth below, it shall have its ordinary meaning in the art.

The term “excipient” as used herein means an inert or inactive substance that may be used in the production of a drug or pharmaceutical, such as a tablet containing a compound of the present disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The terms “individual”, “subject” and “patient” refer to mammals and includes humans and non-human mammals. Examples of patients include, but are not limited to, mice, rats, hamsters, guinea pigs, pigs, rabbits, cats, dogs, goats, sheep, cows, and humans. In some embodiments, patient refers to a human.

As used herein, the term “mammal” includes, but is not limited to, humans, mice, rats, guinea pigs, monkeys, dogs, cats, horses, cows, pigs, and sheep.

“Pharmaceutically acceptable” refers to safe and non-toxic, and suitable for in vivo or for human administration.

As used herein, the term “alkyl”, by itself or as part of another substituent, means, unless otherwise stated, a straight or branched chain hydrocarbon radical, having the number of carbon atoms designated (e.g., C₁-C₆ means one to six carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. In some embodiments, the term “alkyl” may encompass C₁-C₆ alkyl, C₂-C₆ alkyl, C₃-C₆ alkyl, C₄-C₆ alkyl, C₅-C₆ alkyl, C₁-C₅ alkyl, C₂-C₅ alkyl, C₃-C₅ alkyl, C₄-C₅ alkyl, C₁-C₄ alkyl, C₂-C₄ alkyl, C₃-C₄ alkyl, C₁-C₃ alkyl, C₂-C₃ alkyl, or C₁-C₂ alkyl.

The term “cycloalkyl,” “carbocyclic,” or “carbocycle” refers to hydrocarbon rings having the indicated number of ring atoms (e.g., C₃-C₆ cycloalkyl means 3-6 carbons) and being fully saturated or having no more than one double bond between ring vertices. As used herein, “cycloalkyl,” “carbocyclic,” or “carbocycle” is also meant to refer to bicyclic, polycyclic and spirocyclic hydrocarbon rings such as, for example, bicyclo [2.2.1]heptane, pinane, bicyclo [2.2.2]octane, adamantane, norborene, spirocyclic C₅₋₁₂ alkane, etc. In some embodiments, “cycloalkyl” encompasses C₃-C₇ cycloalkyl, C₄-C₇ cycloalkyl, C₅-C₇ cycloalkyl, C₅-C₇ cycloalkyl, C₃-C₆ cycloalkyl, C₄-C₆ cycloalkyl, C₅-C₆ cycloalkyl, C₃-C₅ cycloalkyl, C₄-C₅ cycloalkyl, or C₃-C₄ cycloalkyl.

The term “heteroalkyl,” by itself or in combination with another term, means, unless otherwise stated, a stable straight or branched chain hydrocarbon radical, consisting of the stated number of carbon atoms and from one to three heteroatoms selected from the group consisting of O, N, Si and S, and wherein the nitrogen and sulfur atoms can optionally be oxidized and the nitrogen heteroatom can optionally be quaternized. The heteroatom(s) O, N and S can be placed at any interior position of the heteroalkyl group. The heteroatom Si can be placed at any position of the heteroalkyl group, including the position at which the alkyl group is attached to the remainder of the molecule. A “heteroalkyl” can contain up to three units of unsaturation, and also include mono- and poly-halogenated variants, or combinations thereof. Examples include —CH₂—CH₂—O—CH₃, —CH₂—CH₂—O—CF₃, —CH₂—CH₂—NH—CH₃, —CH₂—CH₂—N(CH₃)—CH₃, —CH₂—S—CH₂—CH₃, —S(O)—CH₃, —CH₂—CH₂—S(O)₂—CH₃, —CH═CHO—CH₃, —Si(CH₃)₃, —CH₂—CH═N—OCH₃, and —CH═CH═N(CH₃)—CH₃. Up to two heteroatoms can be consecutive, such as, for example, —CH₂—NH—OCH₃ and —CH₂—O—Si(CH₃)₃.

The term “heterocycloalkyl,” “heterocyclic,” or “heterocycle” refers to a cycloalkyl radical group having the indicated number of ring atoms (e.g., 5-6 membered heterocycloalkyl) that contain from one to five heteroatoms selected from the group consisting of N, O, and S, wherein the nitrogen and sulfur atoms are optionally oxidized, nitrogen atom(s) are optionally quaternized, as ring atoms. Unless otherwise stated, a “heterocycloalkyl,” “heterocyclic,” or “heterocycle” ring can be a monocyclic, a bicyclic, spirocyclic or a polycylic ring system. Non-limiting examples of “heterocycloalkyl,” “heterocyclic,” or “heterocycle” rings include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-5-oxide, thiomorpholine-S,S-oxide, piperazine, pyran, pyridone, 3-pyrroline, thiopyran, pyrone, tetrahydrofuran, tetrhydrothiophene, quinuclidine, tropane and the like. A “heterocycloalkyl,” “heterocyclic,” or “heterocycle” group can be attached to the remainder of the molecule through one or more ring carbons or heteroatoms. In some embodiments, “heterocycloalkyl” encompasses 4- to 8-membered heterocycloalkyl, 5- to 8-membered heterocycloalkyl, 6- to 8-membered heterocycloalkyl, 7- to 8-membered heterocycloalkyl, 4- to 7-membered heterocycloalkyl, 5- to 7-membered heterocycloalkyl, 6- to 7-membered heterocycloalkyl, 4- to 6-membered heterocycloalkyl, 5- to 6-membered heterocycloalkyl, or 4- to 5-membered heterocycloalkyl.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms. In some embodiments, an alkyl (or alkylene) group will have 10 or fewer carbon atoms.

The term “heteroalkylene” by itself or as part of another substituent means a divalent radical, saturated or unsaturated or polyunsaturated, derived from heteroalkyl, as exemplified by —CH₂—CH₂—S—CH₂CH₂—, —CH₂—S—CH₂—CH₂—NH—CH₂—, —O—CH₂—CH═CH—, —CH₂—CH═C(H)CH₂—O—CH₂— and —S—CH₂—C≡C—. For heteroalkylene groups, heteroatoms can also occupy either or both of the chain termini (e.g., alkyleneoxy, alkylenedioxy, alkyleneamino, alkylenediamino, and the like).

The term “heterocycloalkylene” by itself or as part of another substituent means a divalent radical, saturated or unsaturated or polyunsaturated, derived from heterocycloalkyl. For heterocycloalkylene groups, heteroatoms can also occupy either or both of the chain termini.

The terms “alkoxy” and “alkylamino” are used in their conventional sense, and refer to those alkyl groups attached to the remainder of the molecule via an oxygen atom or an amino group, respectively.

The term “heterocycloalkoxy” refers to a heterocycloalkyl-O— group in which the heterocycloalkyl group is as previously described herein.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “C₁-C₄haloalkyl” is mean to include trifluoromethyl, 2,2,2-trifluoroethyl, 4-chlorobutyl, 3-bromopropyl, difluoromethyl, and the like.

The term “aryl” means, unless otherwise stated, a polyunsaturated, typically aromatic, hydrocarbon group, which can be a single ring or multiple rings (up to three rings) which are fused together. In some embodiments, “aryl” encompasses C₆-C₁₄ aryl, C₅-C₁₄ aryl, C₁₀-C₁₄ aryl, C₁₂-C₁₄ aryl, C₆-C₁₂ aryl, C₈-C₁₂ aryl, C₁₀-C₁₂ aryl, C₆-C₁₁ aryl, C₅-C₁₀ aryl, or C₆-C₈ aryl. The term “heteroaryl” refers to aryl groups (or rings) that contain from one to five heteroatoms selected from the group consisting of N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl groups include phenyl, naphthyl and biphenyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. In some embodiments, the term “heteroaryl” encompasses 5- to 10-membered heteroaryl, 6- to 10-membered heteroaryl, 7- to 10-membered heteroaryl, 8- to 10-membered heteroaryl, 9- to 10-membered heteroaryl, 5- to 9-membered heteroaryl, 6- to 9-membered heteroaryl, 7- to 9-membered heteroaryl, 8- to 9-membered heteroaryl, 5- to 8-membered heteroaryl, 6- to 8-membered heteroaryl, 7- to 8-membered heteroaryl, 5- to 7-membered heteroaryl, 6- to 7-membered heteroaryl, or 5- to 6-membered heteroaryl.

The above terms (e.g., “alkyl,” “aryl” and “heteroaryl”), in some embodiments, will include both substituted and unsubstituted forms of the indicated radical.

As used herein, the term “heteroatom” is meant to include oxygen (O), nitrogen (N), sulfur (S) and silicon (Si).

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution, but differ with regard to the arrangement of the atoms or groups in space.

As used herein, a wavy line “ [symbol] ” that intersects a bond in a chemical structure indicates the point of attachment of the atom to which the wavy bond is connected in the chemical structure to the remainder of a molecule, or to the remainder of a fragment of a molecule.

As used herein, the representation of a group (e.g., X^{a}) in parenthesis followed by a subscript integer range (e.g., (X^{a})₀₋₁) means that the group can have the number of occurrences as designated by the integer range. For example, (X^{a})₀₋₁ means the group X^{a} can be absent or can occur one time.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the present disclosure can contain asymmetric or chiral centers, and therefore exist in different stereoisomeric forms. It is intended that all stereoisomeric forms of the compounds of the present disclosure, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

As used herein, the term “solvate” refers to an association or complex of one or more solvent molecules and a compound of the present disclosure. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water. Certain compounds of the present disclosure can exist in unsolvated forms as well as solvated forms, including hydrated forms. In general, the solvated forms are equivalent to unsolvated forms and are intended to be encompassed within the scope of the present disclosure.

The term “co-crystal” as used herein refers to a solid that is a crystalline single phase material composed of two or more different molecular or ionic compounds generally in a stoichiometric ratio which are neither solvates nor simple salts. A co-crystal consists of two or more components that form a unique crystalline structure having unique properties. Co-crystals are typically characterized by a crystalline structure, which is generally held together by freely reversible, non-covalent interactions. As used herein, a co-crystal refers to a compound of the present disclosure and at least one other component in a defined stoichiometric ratio that form a crystalline structure.

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein, the term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

Certain compounds of the present disclosure possess asymmetric carbon atoms (optical centers) or double bonds; the racemates, diastereomers, geometric isomers, regioisomers and individual isomers (e.g., separate enantiomers) are all intended to be encompassed within the scope of the present disclosure.

The compounds of the present disclosure can also contain unnatural proportions of atomic isotopes at one or more of the atoms that constitute such compounds. For example, the present disclosure also embraces isotopically-labeled variants of the present disclosure which are identical to those recited herein, but for the fact that one or more atoms are replaced by an atom having the atomic mass or mass number different from the predominant atomic mass or mass number usually found in nature for the atom. All isotopes of any particular atom or element as specified are contemplated within the scope of the compounds of the present disclosure and include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically labeled compounds of the present disclosure (e.g., those labeled with ³H or ¹⁴C) are useful in compound and/or substrate tissue distribution assays. Tritiated (³H) and carbon-14 (¹⁴C) isotopes are useful for their ease of preparation and detectability. Further substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present disclosure can generally be prepared by following procedures analogous to those disclosed in the Schemes and/or in the Examples herein below, by substituting an isotopically labeled reagent for a non-isotopically labeled reagent.

“Treating” or “treatment” of a disease in a patient refers to inhibiting the disease or arresting its development; or ameliorating or causing regression of the disease. As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the present disclosure contemplate any one or more of these aspects of treatment.

“Preventing”, “prevention”, or “prophylaxis” of a disease in a patient refers to preventing the disease from occurring in a patient that is predisposed or does not yet display symptoms of the disease.

The phrase “therapeutically effective amount” means an amount of a compound of the present disclosure that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The terms “cancer” and “cancerous” refer to or describe the physiological condition in mammals that is typically characterized by unregulated cell growth.

It is appreciated that certain features of the present disclosure, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present invention and are disclosed herein just as if each and every such sub-combination of chemical groups was individually and explicitly disclosed herein.

### II. Compounds

In one aspect, provided herein is a compound of formula (I)

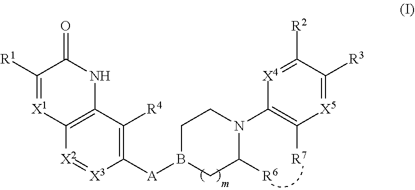

or a pharmaceutically acceptable salt, solvate, hydrate, or co-crystal thereof, or a mixture of any of the foregoing, wherein:
   X¹, X² and X³ are each independently N or C—R¹;
   X⁴ and X⁵ are each independently N or CH;
   A is —S(═O)₂—, —S(═O)(═NH)— or —S(═O)(═N—CH₃)—;
   B is N or CH;
   R¹ is H, halogen, C₁-C₄ alkyl, C₁-C₄ fluoroalkyl, C₁-C₄ alkoxy, or C₃-C₆ cycloalkyl;
   R² is H, halogen, C₁-C₄ alkyl, or C₁-C₄ fluoroalkyl;
   R³ is halogen or —C(═O)NH—R⁵;
   R⁴ is H, halogen, C₁-C₄ alkyl, or C₁-C₄ fluoroalkyl; and
   each R⁵ is independently H, CD₃, C₁-C₄ alkyl, C₁-C₄ fluoroalkyl, C₁₋₄ alkoxy, C₃-C₆ cycloalkyl, or 4- to 7-membered heterocycloalkyl;
   R⁶ is H; and
   R⁷ is H, halogen, CN, C₁-C₄ alkyl, C₁-C₄ fluoroalkyl, C₁-C₄ alkoxy, or C₃-C₆ cycloalkyl; or
   R⁶ and R⁷ are taken together with the atoms to which they are attached to form a ring, wherein R⁶ is —(CH₂)ₙ— and R⁷ is E, wherein E is —O—, —S—, —NR⁵—, or —CR⁵—;
   m is an integer of 1 or 2, wherein when R⁶ is H, then m is 1; and
   n is an integer of 1 or 2.

In some embodiments, the compound of formula (I) is a compound of formula (I-1), or a pharmaceutically acceptable salt, solvate, hydrate, or co-crystal thereof, or a mixture of any of the foregoing,

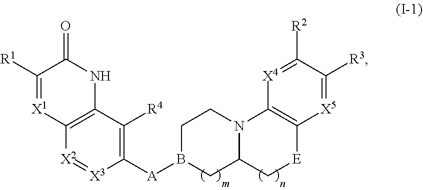

wherein:
E is —O—, —S—, —NR⁵—, or —CR⁵—;
m is an integer of 1 or 2; and
n is an integer of 1 or 2.

In other embodiments, the compound of formula (I) is a compound of formula (I-1), or a pharmaceutically acceptable salt, solvate, hydrate, or co-crystal thereof, or a mixture of any of the foregoing,

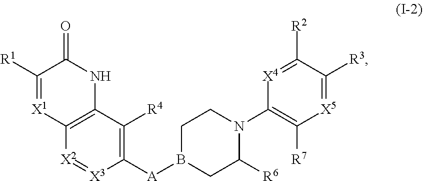

wherein:
R⁶ is H; and
R⁷ is H, halogen, CN, C₁-C₄ alkyl, C₁-C₄ fluoroalkyl, C₁-C₄ alkoxy, or C₃-C₆ cycloalkyl.

In some embodiments, A is —S(═O)₂—, —S(═O)(═NH)— or —S(═O)(═N—CH₃)—. In some embodiments, A is —S(═O)₂— or —S(═O)(═NH)—. In some embodiments, A is —S(═O)(═NH)— or —S(═O)(═N—CH₃)—. In some embodiments, A is —S(═O)₂— or —S(═O)(═N—CH₃)—. In some embodiments, A is —S(═O)₂—. In some embodiments, A is —S(═O)(═NH)—. In some embodiments, A is —S(═O)(═N—CH₃)—.

In some embodiments, B is N or CH. In some embodiments, B is N. In other embodiments, B is CH.

In some embodiments, A is —S(═O)₂—, —S(═O)(═NH)— or —S(═O)(═N—CH₃)— and B is N. In some embodiments, A is —S(═O)₂—, —S(═O)(═NH)— or —S(═O)(═N—CH₃)— and B is CH. In some embodiments, A is —S(═O)₂— and B is N or CH. In some embodiments, A is S(═O)(═NH)— and B is N or CH. In some embodiments, A is —S(═O)(═N—CH₃)— and B is N or CH. In some embodiments, A is —S(═O)₂— and B is N. In some embodiments, A is —S(═O)₂— and B is CH. In some embodiments, A is —S(═O)(═NH)— and B is N. In some embodiments, A is —S(═O)(═NH)— and B is CH. In some embodiments, A is —S(═O)(═N—CH₃)— and B is N. In some embodiments, A is —S(═O)(═N—CH₃)— and B is CH.

In some embodiments, X¹ is N or C—R¹. In some embodiments, X¹ is N or CH. In some embodiments, X¹ is N. In some embodiments, X¹ is C—R¹. In some embodiments, X¹ is CH. In some embodiments, X² is N or C—R¹. In some embodiments, X² is N or CH. In some embodiments, X² is N. In some embodiments, X² is C—R¹. In some embodiments, X² is CH. In some embodiments, X¹ is N and X² is N. In some embodiments, X¹ is CH and X² is CH. In some embodiments, one of X¹ and X² is N and the other of X¹ and X² is CH. In some embodiments, X¹ is N and X² is CH. In some embodiments, X¹ is CH and X² is N. In some embodiments, X³ is N or C—R¹. In some embodiments, X³ is N, CH or CF. In some embodiments, X³ is N. In some embodiments, X³ is C—R¹. In some embodiments, X³ is CH or CF. In some embodiments, X³ is CH. In some embodiments, X³ is CF.

In some embodiments, X⁴ is N or CH. In some embodiments, X⁴ is N. In some embodiments, X⁴ is CH. In some embodiments, X⁵ is N or CH. In some embodiments, X⁵ is N. In some embodiments, X⁵ is CH.

In some embodiments, one of X¹ and X² is N and the other of X¹ and X² is C—R¹; X³ is C—R¹; X⁴ is CH; and X⁵ is N or CH. In some embodiments, one of X¹ and X² is N and the other of X¹ and X² is CH; X³ is CH or CF; X⁴ is CH; and X⁵ is N or CH.

In some embodiments, each R¹ is independently H, halogen, C₁-C₄ alkyl, C₁-C₄ fluoroalkyl, C₁-C₄ alkoxy, or C₃-C₆ cycloalkyl. In some embodiments, each R¹ is independently H, halogen, C₁-C₄ alkyl, C₁-C₄ fluoroalkyl, or C₁-C₄ alkoxy. In some embodiments, each R¹ is independently H, halogen, C₁-C₄ alkyl, C₁-C₄ alkoxy, or C₃-C₆ cycloalkyl. In some embodiments, each R¹ is independently H, halogen, C₁-C₄ fluoroalkyl, C₁-C₄ alkoxy, or C₃-C₆ cycloalkyl. In some embodiments, each R¹ is independently halogen, C₁-C₄ alkyl, C₁-C₄ fluoroalkyl, C₁-C₄ alkoxy, or C₃-C₆ cycloalkyl. In some embodiments, each R¹ is independently H, halogen, C₁-C₄ alkyl, or C₁-C₄ fluoroalkyl. In some embodiments, each R¹ is independently C₁-C₄ alkyl, C₁-C₄ fluoroalkyl, C₁-C₄ alkoxy, or C₃-C₆ cycloalkyl. In some embodiments, each R¹ is independently C₁-C₄ alkyl, C₁-C₄ fluoroalkyl, or C₁-C₄ alkoxy. In some embodiments, each R¹ is independently C₁-C₄ fluoroalkyl, C₁-C₄ alkoxy, or C₃-C₆ cycloalkyl. In some embodiments, each R¹ is independently H, halogen or C₁-C₄ alkyl. In some embodiments, each R¹ is independently H or halogen. In some embodiments, each R¹ is independently halogen or C₁-C₄ alkyl. In some embodiments, each R¹ is independently halogen or C₁-C₄ fluoroalkyl. In some embodiments, each R¹ is independently H, C₁-C₄ alkyl, C₁-C₄ fluoroalkyl, or C₁-C₄ alkoxy. In some embodiments, each R¹ is independently H, C₁-C₄ alkyl, C₁-C₄ fluoroalkyl, or C₁-C₄ alkoxy. In some embodiments, each R¹ is independently H, C₁-C₄ alkyl, or C₁-C₄ fluoroalkyl. In some embodiments, each R¹ is independently H, C₁-C₄ alkyl, or C₁-C₄ alkoxy. In some embodiments, each R¹ is independently C₁-C₄ alkyl, C₁-C₄ fluoroalkyl, or C₁-C₄ alkoxy. In some embodiments, each R¹ is independently H or C₁-C₄ alkyl. In some embodiments, each R¹ is independently C₁-C₄ alkyl or C₁-C₄ fluoroalkyl. In some embodiments, each R¹ is independently C₁-C₄ alkyl or C₁-C₄ alkoxy. In some embodiments, each R¹ is independently C₁-C₄ fluoroalkyl or C₁-C₄ alkoxy.

In some embodiments, R¹ is H.

In some embodiments, R¹ is halogen. In some embodiments wherein R¹ is halogen, R¹ is F or Cl. In some embodiments, R¹ is F. In some embodiments, R¹ is Cl.

In some embodiments, R¹ is C₁-C₄ alkyl. In some embodiments, R¹ is C₁-C₃ alkyl. In some embodiments, R¹ is C₂-C₄ alkyl. In some embodiments, R¹ is C₁-C₂ alkyl. In some embodiments wherein R¹ is C₁-C₄ alkyl, R¹ is —CH₃, —CH₂CH₃, —(CH₂)₂CH₃, —CH₂(CH₃)₂, —(CH₂)₃CH₃, —(CH₂)₃CH₃, —CH₂CH(CH₃)₂, —CH(CH₃)CH₂CH₃, or —CH(CH₃)₃. In some embodiments, R¹ is —CH₃ or —CH₂CH₃. In some embodiments, R¹ is —CH₃. In some embodiments, R¹ is —CH₂CH₃.

In some embodiments, R¹ is C₁-C₄ fluoroalkyl. In some embodiments wherein R¹ is C₁-C₄ fluoroalkyl, R¹ is C₁-C₄ fluoroalkyl wherein the aliphatic portions of R¹ are substituted by from 1 to 5 fluorine atoms. In some embodiments, R¹ is a C₁-C₃ fluoroalkyl. In some embodiments, R¹ is a C₁-C₂ fluoroalkyl. In some embodiments, R¹ is a C₂-C₄ fluoroalkyl. In some embodiments, R¹ is —CH₂F, —CHF₂, —CF₃, —CHFCH₃, —CH₂CH₂F, —CH₂CHF₂, —CF₂CH₃, —CH₂CF₃, —CHFCH₂F, —CF₂CF₃, or —(CH₂)₂CH₃. In some embodiments, R¹ is —CF₃ or —CH₂CF₃. In some embodiments, R¹ is —CF₃. In some embodiments, R¹ is —CH₂CF₃.

In some embodiments, R¹ is C₁-C₄ alkoxy. In some embodiments, R¹ is C₁-C₃ alkoxy. In some embodiments, R¹ is C₂-C₄ alkoxy. In some embodiments, R¹ is a C₁-C₂ alkoxy. In some embodiments, R¹ is —OCH₃, —OCH₂CH₃, —O(CH₂)₂CH₃, —OCH₂(CH₃)₂, —O(CH₂)₃CH₃, —O(CH₂)₃CH₃, —OCH₂CH(CH₃)₂, —OCH(CH₃)CH₂CH₃, or —OCH(CH₃)₃. In some embodiments, R¹ is —OCH₃ or —OCH₂CH₃. In some embodiments, R¹ is —OCH₃. In some embodiments, R¹ is —OCH₂CH₃.

In some embodiments, R¹ is C₃-C₆ cycloalkyl. In some embodiments, R¹ is C₃-C₅ cycloalkyl. In some embodiments, R¹ is C₃-C₄ cycloalkyl. In some embodiments, R¹ is C₄-C₆ cycloalkyl. In some embodiments, R¹ is C₄-C₅ cycloalkyl. In some embodiments, R¹ is C₅-C₆ cycloalkyl. In some embodiments, R¹ is cyclopropyl, cyclobutyl, cyclopentyl or cyclohexyl. In some embodiments, R¹ is cyclopropyl, cyclobutyl, or cyclopentyl. In some embodiments, R¹ is cyclopropyl or cyclobutyl. In some embodiments, R¹ is cyclobutyl, cyclopentyl or cyclohexyl. In some embodiments, R¹ is cyclobutyl or cyclopentyl. In some embodiments, R¹ is cyclopentyl or cyclohexyl. In some embodiments, R¹ is cyclopropyl. In some embodiments, R¹ is cyclobutyl. In some embodiments, R¹ is cyclopentyl. In some embodiments, R¹ is cyclohexyl.

In some embodiments, R² is H, halogen, C₁-C₄ alkyl, or C₁-C₄ fluoroalkyl. In some embodiments, R² is H, halogen, or C₁-C₄ alkyl. In some embodiments, R² is H, C₁-C₄ alkyl, or C₁-C₄ fluoroalkyl. In some embodiments, R² is H, halogen, or C₁-C₄ fluoroalkyl. In some embodiments, R² is halogen, C₁-C₄ alkyl, or C₁-C₄ fluoroalkyl. In some embodiments, R² is H or halogen. In some embodiments, R² is H or C₁-C₄ alkyl. In some embodiments, R² is H or C₁-C₄ fluoroalkyl. In some embodiments, R² is halogen or C₁-C₄ alkyl. In some embodiments, R² is halogen or C₁-C₄ fluoroalkyl. In some embodiments, R² is C₁-C₄ alkyl or C₁-C₄ fluoroalkyl. In some embodiments, R² is H.

In some embodiments, R² is halogen. In some embodiments wherein R² is halogen, R² is F or Cl. In some embodiments, R² is F. In some embodiments, R² is Cl.

In some embodiments, R² is C₁-C₄ alkyl. In some embodiments, R² is C₁-C₃ alkyl. In some embodiments, R² is C₂-C₄ alkyl. In some embodiments, R² is C₁-C₂ alkyl. In some embodiments wherein R² is C₁-C₄ alkyl, R² is —CH₃, —CH₂CH₃, —(CH₂)₂CH₃, —CH₂(CH₃)₂, —(CH₂)₃CH₃, —(CH₂)₃CH₃, —CH₂CH(CH₃)₂, —CH(CH₃)CH₂CH₃, or —CH(CH₃)₃. In some embodiments, R² is —CH₃ or —CH₂CH₃. In some embodiments, R² is —CH₃. In some embodiments, R² is —CH₂CH₃.

In some embodiments, R² is C₁-C₄ fluoroalkyl. In some embodiments wherein R² is C₁-C₄ fluoroalkyl, R² is C₁-C₄ fluoroalkyl wherein the aliphatic portions of R² are substituted by from 1 to 5 fluorine atoms. In some embodiments, R² is a C₁-C₃ fluoroalkyl. In some embodiments, R² is a C₁-C₂ fluoroalkyl. In some embodiments, R² is a C₂-C₄ fluoroalkyl. In some embodiments, R² is —CH₂F, —CHF₂, —CF₃, —CHFCH₃, —CH₂CH₂F, —CH₂CHF₂, —CF₂CH₃, —CH₂CF₃, —CHFCH₂F, —CF₂CF₃, or —(CH₂)₂CH₃. In some embodiments, R² is —CF₃ or —CH₂CF₃. In some embodiments, R² is —CF₃. In some embodiments, R² is —CH₂CF₃.

In some embodiments, R³ is halogen or —C(═O)NH—R⁵. In some embodiments, R³ is halogen. In some embodiments, R³ is —C(═O)NH—R⁵. In some embodiments wherein R³ is halogen, R³ is F or Cl. In some embodiments, R³ is F. In some embodiments, R³ is Cl. In some embodiments wherein R³ is —C(═O)NH—R⁵, R⁵ is H, C₁-C₄ alkyl, or C₁-C₄ fluoroalkyl. In some embodiments wherein R³ is —C(═O)NH—R⁵, R⁵ is H. In some embodiments wherein R³ is —C(═O)NH—R⁵, R⁵ is C₁-C₄ alkyl. In some embodiments wherein R³ is —C(═O)NH—R⁵, R⁵ is C₁-C₄ fluoroalkyl. In some embodiments wherein R³ is —C(═O)NH—R⁵, R³ is —C(═O)NH—CH₃.

In some embodiments, R⁴ is H, halogen, C₁-C₄ alkyl, or C₁-C₄ fluoroalkyl. In some embodiments, R⁴ is H, halogen, or C₁-C₄ alkyl. In some embodiments, R⁴ is H, C₁-C₄ alkyl, or C₁-C₄ fluoroalkyl. In some embodiments, R⁴ is H, halogen, or C₁-C₄ fluoroalkyl. In some embodiments, R⁴ is halogen, C₁-C₄ alkyl, or C₁-C₄ fluoroalkyl. In some embodiments, R⁴ is H or halogen. In some embodiments, R⁴ is H or C₁-C₄ alkyl. In some embodiments, R⁴ is H or C₁-C₄ fluoroalkyl. In some embodiments, R⁴ is halogen or C₁-C₄ alkyl. In some embodiments, R⁴ is halogen or C₁-C₄ fluoroalkyl. In some embodiments, R⁴ is C₁-C₄ alkyl or C₁-C₄ fluoroalkyl. In some embodiments, R⁴ is H.

In some embodiments, R⁴ is halogen. In some embodiments wherein R⁴ is halogen, R⁴ is F or Cl. In some embodiments, R⁴ is F. In some embodiments, R⁴ is Cl.

In some embodiments, R⁴ is C₁-C₄ alkyl. In some embodiments, R⁴ is C₁-C₃ alkyl. In some embodiments, R⁴ is C₂-C₄ alkyl. In some embodiments, R⁴ is C₁-C₂ alkyl. In some embodiments wherein R⁴ is C₁-C₄ alkyl, R⁴ is —CH₃, —CH₂CH₃, —(CH₂)₂CH₃, —CH₂(CH₃)₂, —(CH₂)₃CH₃, —(CH₂)₃CH₃, —CH₂CH(CH₃)₂, —CH(CH₃)CH₂CH₃, or —CH(CH₃)₃. In some embodiments, R⁴ is —CH₃ or —CH₂CH₃. In some embodiments, R⁴ is —CH₃. In some embodiments, R⁴ is —CH₂CH₃.

In some embodiments, R⁴ is C₁-C₄ fluoroalkyl. In some embodiments wherein R⁴ is C₁-C₄ fluoroalkyl, R⁴ is C₁-C₄ fluoroalkyl wherein the aliphatic portions of R⁴ are substituted by from 1 to 5 fluorine atoms. In some embodiments, R⁴ is a C₁-C₃ fluoroalkyl. In some embodiments, R⁴ is a C₁-C₂ fluoroalkyl. In some embodiments, R⁴ is a C₂-C₄ fluoroalkyl. In some embodiments, R⁴ is —CH₂F, —CHF₂, —CF₃, —CHFCH₃, —CH₂CH₂F, —CH₂CHF₂, —CF₂CH₃, —CH₂CF₃, —CHFCH₂F, —CF₂CF₃, or —(CH₂)₂CH₃. In some embodiments, R⁴ is —CF₃ or —CH₂CF₃. In some embodiments, R⁴ is —CF₃. In some embodiments, R⁴ is —CH₂CF₃.

In some embodiments, each R⁵ is independently H, CD₃, C₁-C₄ alkyl, C₁-C₄ fluoroalkyl, C₁₋₄ alkoxy, C₃-C₆ cycloalkyl, or 4- to 7-membered heterocycloalkyl. In some embodiments, each R⁵ is independently H, CD₃, C₁-C₄ alkyl, C₁-C₄ fluoroalkyl, or C₁₋₄ alkoxy. In some embodiments, each R⁵ is independently H, CD₃, C₁-C₄ alkyl, or C₁-C₄ fluoroalkyl. In some embodiments, each R⁵ is independently H, CD₃, C₁-C₄ alkyl, or C₁₋₄ alkoxy. In some embodiments, each R⁵ is independently H, CD₃, or C₁-C₄ alkyl. In some embodiments, each R⁵ is independently C₁-C₄ alkyl, C₁-C₄ fluoroalkyl, or C₁₋₄ alkoxy. In some embodiments, each R⁵ is independently C₁-C₄ fluoroalkyl, C₁₋₄ alkoxy, or C₃-C₆ cycloalkyl. In some embodiments, each R⁵ is independently C₁-C₄ fluoroalkyl, C₁₋₄ alkoxy, C₃-C₆ cycloalkyl, or 4- to 7-membered heterocycloalkyl. In some embodiments, each R⁵ is independently C₁₋₄ alkoxy, C₃-C₆ cycloalkyl, or 4- to 7-membered heterocycloalkyl. In some embodiments, each R⁵ is independently C₃-C₆ cycloalkyl or 4- to 7-membered heterocycloalkyl. In some embodiments, each R⁵ is independently H, C₁-C₄ alkyl, or C₁-C₄ fluoroalkyl. In some embodiments, each R⁵ is independently H or C₁-C₄ alkyl. In some embodiments, each R⁵ is independently H or C₁-C₄ fluoroalkyl. In some embodiments, each R⁵ is independently C₁-C₄ alkyl or C₁-C₄ fluoroalkyl. In some embodiments R⁵ is H.

In some embodiments, R⁵ is C₁-C₄ alkyl. In some embodiments, R⁵ is C₁-C₃ alkyl. In some embodiments, R⁵ is C₂-C₄ alkyl. In some embodiments, R⁵ is C₁-C₂ alkyl. In some embodiments wherein R⁵ is C₁-C₄ alkyl, R⁵ is —CH₃, —CH₂CH₃, —(CH₂)₂CH₃, —CH₂(CH₃)₂, —(CH₂)₃CH₃, —(CH₂)₃CH₃, —CH₂CH(CH₃)₂, —CH(CH₃)CH₂CH₃, or —CH(CH₃)₃. In some embodiments, R⁵ is —CH₃ or —CH₂CH₃. In some embodiments, R⁵ is —CH₃. In some embodiments, R⁵ is —CH₂CH₃.

In some embodiments, R⁵ is C₁-C₄ fluoroalkyl. In some embodiments, R⁵ is C₁-C₄ fluoroalkyl. In some embodiments wherein R⁵ is C₁-C₄ fluoroalkyl, R⁵ is C₁-C₄ fluoroalkyl wherein the aliphatic portions of R⁵ are substituted by from 1 to 5 fluorine atoms. In some embodiments, R⁵ is a C₁-C₃ fluoroalkyl. In some embodiments, R⁵ is a C₁-C₂ fluoroalkyl. In some embodiments, R⁵ is a C₂-C₄ fluoroalkyl. In some embodiments, R⁵ is —CH₂F, —CHF₂, —CF₃, —CHFCH₃, —CH₂CH₂F, —CH₂CHF₂, —CF₂CH₃, —CH₂CF₃, —CHFCH₂F, —CF₂CF₃, or —(CH₂)₂CH₃. In some embodiments, R⁵ is —CF₃ or —CH₂CF₃. In some embodiments, R⁵ is —CF₃. In some embodiments, R⁵ is —CH₂CF₃.

In some embodiments, R⁵ is C₁-C₄ alkoxy. In some embodiments, R⁵ is C₁-C₃ alkoxy. In some embodiments, R⁵ is C₂-C₄ alkoxy. In some embodiments, R⁵ is a C₁-C₂ alkoxy. In some embodiments, R⁵ is —OCH₃, —OCH₂CH₃, —O(CH₂)₂CH₃, —OCH₂(CH₃)₂, —O(CH₂)₃CH₃, —O(CH₂)₃CH₃, —OCH₂CH(CH₃)₂, —OCH(CH₃)CH₂CH₃, or —OCH(CH₃)₃. In some embodiments, R⁵ is —OCH₃ or —OCH₂CH₃. In some embodiments, R⁵ is —OCH₃. In some embodiments, R⁵ is —OCH₂CH₃.

In some embodiments, R⁵ is C₃-C₆ cycloalkyl. In some embodiments, R⁵ is C₃-C₅ cycloalkyl. In some embodiments, R⁵ is C₃-C₄ cycloalkyl. In some embodiments, R⁵ is C₄-C₆ cycloalkyl. In some embodiments, R⁵ is C₄-C₅ cycloalkyl. In some embodiments, R⁵ is C₅-C₆ cycloalkyl. In some embodiments, R⁵ is cyclopropyl, cyclobutyl, cyclopentyl or cyclohexyl. In some embodiments, R⁵ is cyclopropyl, cyclobutyl, or cyclopentyl. In some embodiments, R⁵ is cyclopropyl or cyclobutyl. In some embodiments, R⁵ is cyclobutyl, cyclopentyl or cyclohexyl. In some embodiments, R⁵ is cyclobutyl or cyclopentyl. In some embodiments, R⁵ is cyclopentyl or cyclohexyl. In some embodiments, R⁵ is cyclopropyl. In some embodiments, R⁵ is cyclobutyl. In some embodiments, R⁵ is cyclopentyl. In some embodiments, R⁵ is cyclohexyl.

In some embodiments, R⁵ is 4- to 7-membered heterocycloalkyl. In some embodiments, R⁵ is 4- to 6-membered heterocycloalkyl. In some embodiments, R⁵ is 4- to 5-membered heterocycloalkyl. In some embodiments, R⁵ is 5- to 7-membered heterocycloalkyl. In some embodiments, R⁵ is 5- to 6-membered heterocycloalkyl. In some embodiments, R⁵ is 6- to 7-membered heterocycloalkyl.

In some embodiments, R⁶ is H.

In some embodiments, R⁷ is H, halogen, CN, C₁-C₄ alkyl, C₁-C₄ fluoroalkyl, C₁-C₄ alkoxy, or C₃-C₆ cycloalkyl. In some embodiments, R⁷ is H, halogen, CN, C₁-C₄ alkyl, C₁-C₄ fluoroalkyl, or C₁-C₄ alkoxy. In some embodiments, R⁷ is H, halogen, CN, C₁-C₄ alkyl, C₁-C₄ fluoroalkyl, or C₃-C₆ cycloalkyl. In some embodiments, R⁷ is H, halogen, CN, C₁-C₄ alkyl, C₁-C₄ alkoxy, or C₃-C₆ cycloalkyl. In some embodiments, R⁷ is H, halogen, CN, C₁-C₄ fluoroalkyl, C₁-C₄ alkoxy, or C₃-C₆ cycloalkyl. In some embodiments, R⁷ is H, halogen, C₁-C₄ alkyl, C₁-C₄ fluoroalkyl, C₁-C₄ alkoxy, or C₃-C₆ cycloalkyl. In some embodiments, R⁷ is H, CN, C₁-C₄ alkyl, C₁-C₄ fluoroalkyl, C₁-C₄ alkoxy, or C₃-C₆ cycloalkyl. In some embodiments, R⁷ is halogen, CN, C₁-C₄ alkyl, C₁-C₄ fluoroalkyl, C₁-C₄ alkoxy, or C₃-C₆ cycloalkyl. In some embodiments, R⁷ is halogen, CN, C₁-C₄ alkyl, C₁-C₄ fluoroalkyl, or C₁-C₄ alkoxy. In some embodiments, R⁷ is halogen, CN, C₁-C₄ alkyl, or C₁-C₄ fluoroalkyl. In some embodiments, R⁷ is halogen, CN, or C₁-C₄ alkyl. In some embodiments, R⁷ is halogen or CN. In some embodiments, R⁷ is H, halogen, CN, or C₁-C₄ alkyl. In some embodiments, R⁷ is C₁-C₄ alkyl, C₁-C₄ fluoroalkyl, C₁-C₄ alkoxy, or C₃-C₆ cycloalkyl. In some embodiments, R⁷ is C₁-C₄ alkyl, C₁-C₄ fluoroalkyl, or C₁-C₄ alkoxy.

In some embodiments, R⁷ is H. In some embodiments, R⁷ is halogen. In some embodiments, R⁷ is F. In some embodiments, R⁷ is CN.

In some embodiments, R⁷ is C₁-C₄ alkyl. In some embodiments, R⁷ is C₁-C₃ alkyl. In some embodiments, R⁷ is C₂-C₄ alkyl. In some embodiments, R⁷ is C₁-C₂ alkyl. In some embodiments wherein R⁷ is C₁-C₄ alkyl, R⁷ is —CH₃, —CH₂CH₃, —(CH₂)₂CH₃, —CH₂(CH₃)₂, —(CH₂)₃CH₃, —(CH₂)₃CH₃, —CH₂CH(CH₃)₂, —CH(CH₃)CH₂CH₃, or —CH(CH₃)₃. In some embodiments, R⁷ is —CH₃ or —CH₂CH₃. In some embodiments, R⁷ is —CH₃. In some embodiments, R⁷ is —CH₂CH₃.

In some embodiments, R⁷ is C₁-C₄ fluoroalkyl. In some embodiments, R⁷ is C₁-C₄ fluoroalkyl. In some embodiments wherein R⁷ is C₁-C₄ fluoroalkyl, R⁷ is C₁-C₄ fluoroalkyl wherein the aliphatic portions of R⁷ are substituted by from 1 to 5 fluorine atoms. In some embodiments, R⁷ is a C₁-C₃ fluoroalkyl. In some embodiments, R⁷ is a C₁-C₂ fluoroalkyl. In some embodiments, R⁷ is a C₂-C₄ fluoroalkyl. In some embodiments, R⁷ is —CH₂F, —CHF₂, —CF₃, —CHFCH₃, —CH₂CH₂F, —CH₂CHF₂, —CF₂CH₃, —CH₂CF₃, —CHFCH₂F, —CF₂CF₃, or —(CH₂)₂CH₃. In some embodiments, R⁷ is —CF₃ or —CH₂CF₃. In some embodiments, R⁷ is —CF₃. In some embodiments, R⁷ is —CH₂CF₃.

In some embodiments, R⁷ is C₁-C₄ alkoxy. In some embodiments, R⁷ is C₁-C₃ alkoxy. In some embodiments, R⁷ is C₂-C₄ alkoxy. In some embodiments, R⁷ is a C₁-C₂ alkoxy. In some embodiments, R⁷ is —OCH₃, —OCH₂CH₃, —O(CH₂)₂CH₃, —OCH₂(CH₃)₂, —O(CH₂)₃CH₃, —O(CH₂)₃CH₃, —OCH₂CH(CH₃)₂, —OCH(CH₃)CH₂CH₃, or —OCH(CH₃)₃. In some embodiments, R⁷ is —OCH₃ or —OCH₂CH₃. In some embodiments, R⁷ is —OCH₃. In some embodiments, R⁷ is —OCH₂CH₃.

In some embodiments, R⁷ is C₃-C₆ cycloalkyl. In some embodiments, R⁷ is C₃-C₅ cycloalkyl. In some embodiments, R⁷ is C₃-C₄ cycloalkyl. In some embodiments, R⁷ is C₄-C₆ cycloalkyl. In some embodiments, R⁷ is C₄-C₅ cycloalkyl. In some embodiments, R⁷ is C₅-C₆ cycloalkyl. In some embodiments, R⁷ is cyclopropyl, cyclobutyl, cyclopentyl or cyclohexyl. In some embodiments, R⁷ is cyclopropyl, cyclobutyl, or cyclopentyl. In some embodiments, R⁷ is cyclopropyl or cyclobutyl. In some embodiments, R⁷ is cyclobutyl, cyclopentyl or cyclohexyl. In some embodiments, R⁷ is cyclobutyl or cyclopentyl. In some embodiments, R⁷ is cyclopentyl or cyclohexyl. In some embodiments, R⁷ is cyclopropyl. In some embodiments, R⁷ is cyclobutyl. In some embodiments, R⁷ is cyclopentyl. In some embodiments, R⁷ is cyclohexyl.

In some embodiments, R⁶ is H and R⁷ is H or halogen.

In some embodiments, wherein R⁶ and R⁷ are taken together with the atoms to which they are attached to form a ring, wherein R⁶ is —(CH₂)ₙ— and R⁷ is E, wherein E is —O—, —S—, —NR⁵—, or —CR⁵—.

In some embodiments, m is an integer of 1 or 2, wherein when R⁶ is H, then m is 1. In other embodiments wherein R⁶ and R⁷ are taken together with the atoms to which they are attached to form a ring, m is an integer of 1 or 2. In some embodiments wherein R⁶ and R⁷ are taken together with the atoms to which they are attached to form a ring, m is an integer of 1. In other embodiments wherein R⁶ and R⁷ are taken together with the atoms to which they are attached to form a ring, m is an integer of 2.

In some embodiments wherein R⁶ and R⁷ are taken together with the atoms to which they are attached to form a ring and R⁶ is —(CH₂)ₙ—, n is an integer of 1 or 2. In some embodiments, n is an integer of 1. In other embodiments, n is an integer of 2.

In some embodiments wherein R⁶ and R⁷ are taken together with the atoms to which they are attached to form a ring and R⁷ is E, E is —O—, —S—, —NR⁵—, or —CR⁵—. In some embodiments, E is —O—, —NR⁵— or —CR⁵—. In some embodiments, E is —S—, —NR⁵— or —CR⁵—. In some embodiments, E is —O—, —S—, or —NR⁵—. In some embodiments, E is —S—, —NR⁵— or —CR⁵—. In some embodiments, E is —O— or —S—. In some embodiments, E is —O— or —NR⁵—. In some embodiments, E is —O— or —CR⁵—. In some embodiments, E is —S— or —NR⁵—. In some embodiments, E is —S— or —CR⁵—. In some embodiments, E is —NR⁵— or —CR⁵—. In some embodiments, E is —O—. In some embodiments, E is —S—. In some embodiments, E is —NR⁵—. In some embodiments, E is —CR⁵—.

In some embodiments, the compound is a compound of formula (I-A), (I-B), (I-C), (I-D), (I-E), (I-F), (I-G), or (I-H), or a pharmaceutically acceptable salt, solvate, hydrate, or co-crystal thereof, or a mixture of any of the foregoing:

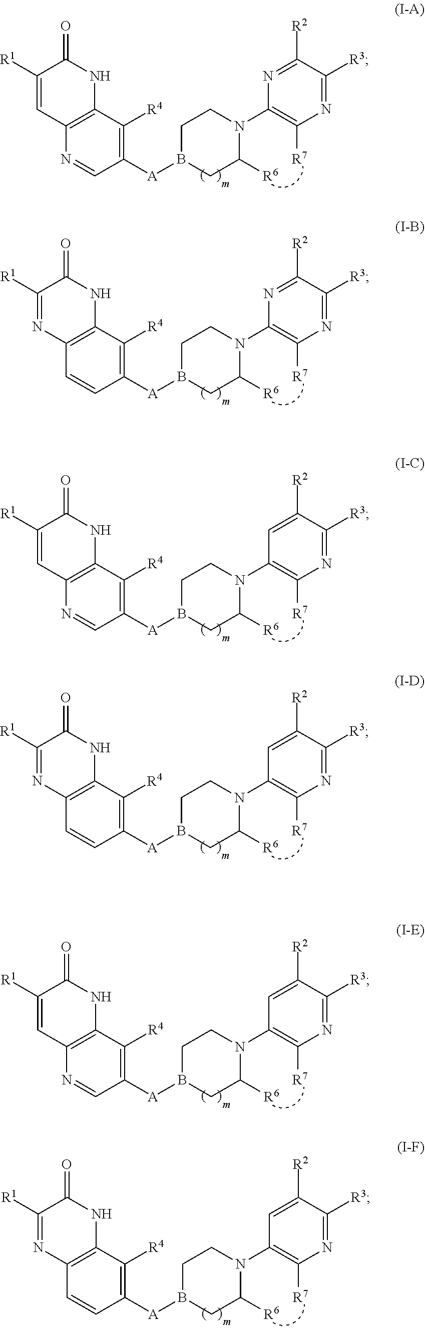

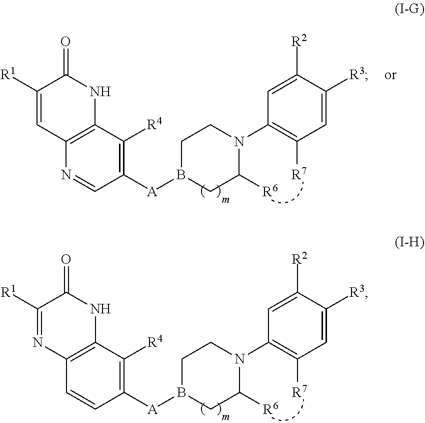

wherein A, B, R¹, R², R³, R⁴, R⁵, R⁶, R⁷, E, m and/or n are as defined for Formula (I). In some embodiments, the compound is a compound of formula (I-A). In some embodiments, the compound is a compound of formula (I-B). In some embodiments, the compound is a compound of formula (I-C). In some embodiments, the compound is a compound of formula (I-D). In some embodiments, the compound is a compound of formula (I-E). In some embodiments, the compound is a compound of formula (I-F). In some embodiments, the compound is a compound of formula (I-G). In some embodiments, the compound is a compound of formula (I-G). In some embodiments, R⁶ is H, m is 1, and R⁷ is H, halogen, CN, C₁-C₄ alkyl, C₁-C₄ fluoroalkyl, C₁-C₄ alkoxy, or C₃-C₆ cycloalkyl. In some embodiments, R⁶ and R⁷ are taken together with the atoms to which they are attached to form a ring, wherein R⁶ is —(CH₂)ₙ— and R⁷ is E, wherein E is —O—, —S—, —NR¹—, or —CR⁵—; m is an integer of 1 or 2, and n is an integer of 1 or 2.

In some embodiments, the compound is a compound of formula (I-1-a), (I-1-b), (I-1-c), (I-1-d), (I-1-e), or (I-1-f), or a pharmaceutically acceptable salt, solvate, hydrate, or co-crystal thereof, or a mixture of any of the foregoing:

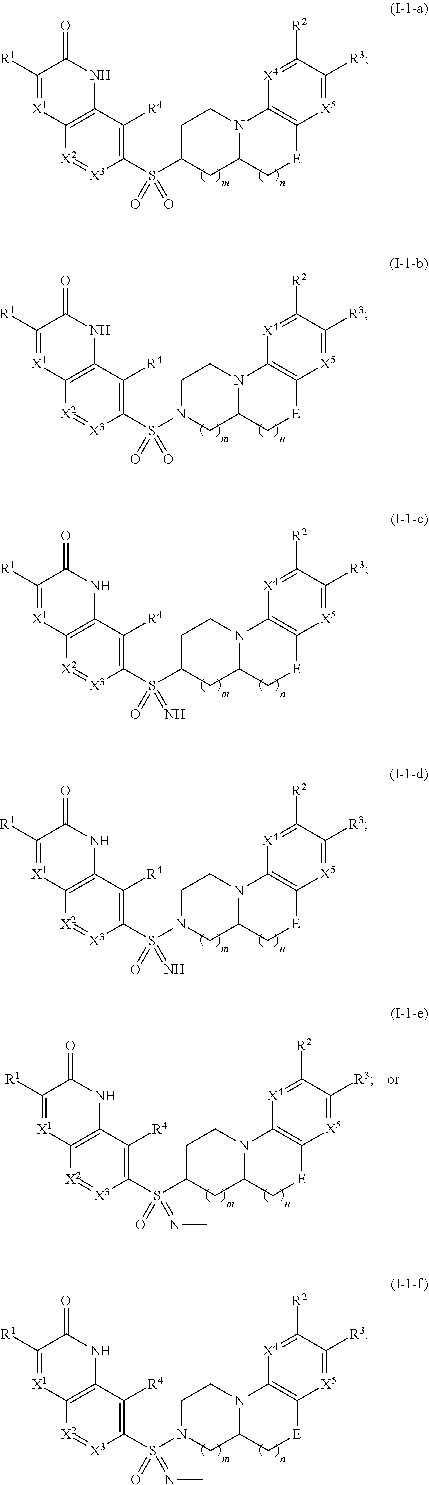

wherein X¹, X², X³, X⁴, X⁵, R₁, R₂, R₃, R₄, R₅, E, m and/or n are as defined for Formula (I) or Formula (I-1). In some embodiments, the compound is a compound of formula (I-1-a). In some embodiments, the compound is a compound of formula (I-1-b). In some embodiments, the compound is a compound of formula (I-1-c). In some embodiments, the compound is a compound of formula (I-1-d). In some embodiments, the compound is a compound of formula (I-1-e). In some embodiments, the compound is a compound of formula (I-1-f).

In other embodiments, the compound is a compound of formula (I-2-a), (I-2-b), (I-2-c), (I-2-d), (I-2-e), or (I-2-f), or a pharmaceutically acceptable salt, solvate, hydrate, or co-crystal thereof, or a mixture of any of the foregoing:

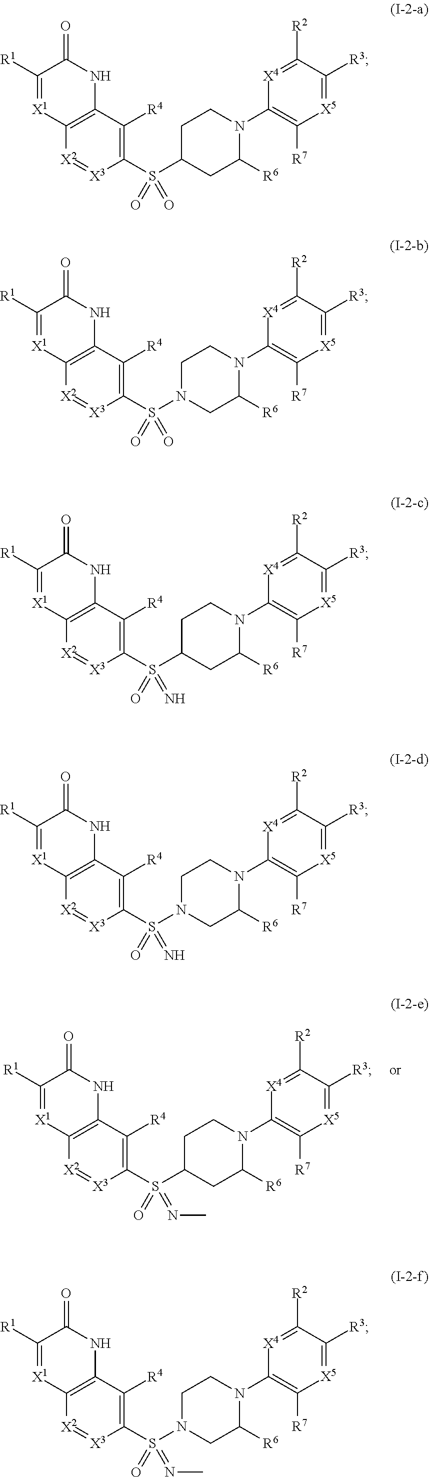

wherein X¹, X², X³, X⁴, X⁵, R¹, R², R³, and R⁴ are as defined for Formula (I) or Formula (I-2). In some embodiments, the compound is a compound of formula (I-2-a). In some embodiments, the compound is a compound of formula (I-2-b). In some embodiments, the compound is a compound of formula (I-2-c). In some embodiments, the compound is a compound of formula (I-2-d). In some embodiments, the compound is a compound of formula (I-2-e). In some embodiments, the compound is a compound of formula (I-2-f).

In some embodiments, the compound is a compound of formula (I-2-a) or (I-2-b), wherein X¹ is N or CH, X² is N or CH, X³ is CH, X⁴ is CH; X⁵ is N or CH, R¹ is C₁-C₃ alkyl, R² is H, R³ is halogen or —C(═O)NH—R⁵, R⁴ is H, R⁶ is H, and R⁷ is H or halogen. In some embodiments, the compound is a compound of formula (I-2-c) or (I-2-d), wherein X¹ is CH, X² is N or CH, X³ is CH, X⁴ is CH; X⁵ is N, R¹ is C₁-C₃ alkyl, R² is H, R³ is halogen or —C(═O)NH—R⁵, R⁴ is H, R⁶ is H, and R⁷ is H or halogen

In some embodiments, provided is a compound selected from the compounds in Table 1, or a pharmaceutically acceptable salt, solvate, hydrate, or co-crystal thereof, or a mixture of any of the foregoing.

TABLE 1

| Cmpd | |
|---|---|
| No. | Structure |
| 2.1 | 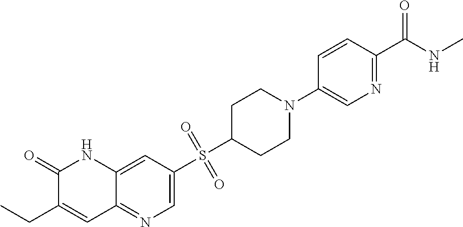 [SMILES: CCc1cc2ncc(S(=O)(=O)C3CCN(c4ccc(C(=O)NC)nc4)CC3)cc2[nH]c1=O] |
| 2.2 | 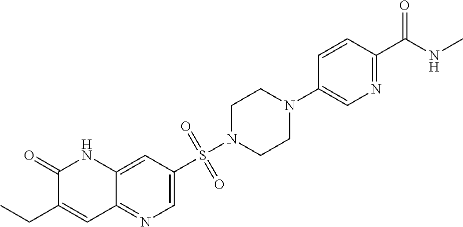 [SMILES: CCc1cc2ncc(S(=O)(=O)N3CCN(c4ccc(C(=O)NC)nc4)CC3)cc2[nH]c1=O] |
| 2.3 | 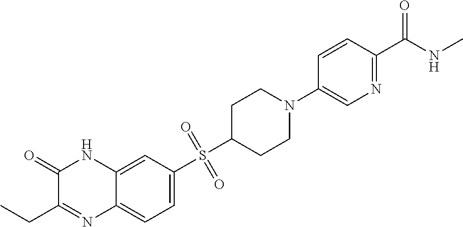 [SMILES: CCc1nc2ccc(S(=O)(=O)C3CCN(c4ccc(C(=O)NC)nc4)CC3)cc2[nH]c1=O] |
| 2.4 | 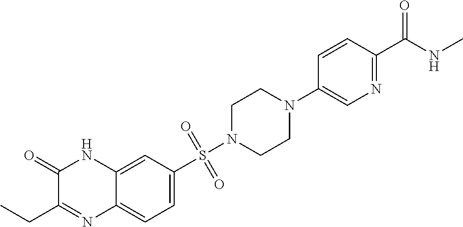 [SMILES: CCc1nc2ccc(S(=O)(=O)N3CCN(c4ccc(C(=O)NC)nc4)CC3)cc2[nH]c1=O] |
| 2.5 | 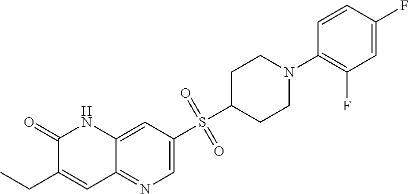 [SMILES: CCc1cc2ncc(S(=O)(=O)C3CCN(c4ccc(F)cc4F)CC3)cc2[nH]c1=O] |
| 2.6 | 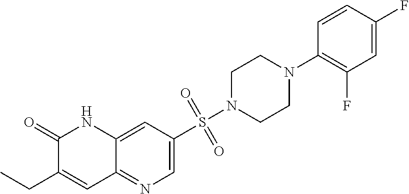 [SMILES: CCc1cc2ncc(S(=O)(=O)N3CCN(c4ccc(F)cc4F)CC3)cc2[nH]c1=O] |
| 2.7 | 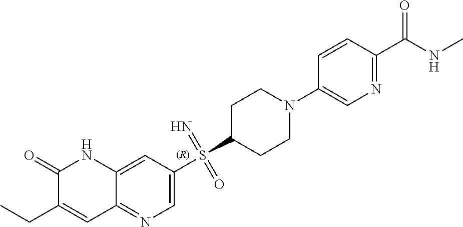 |
| 2.7-1 | 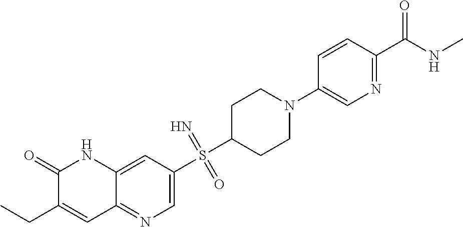 [SMILES: CCc1cc2ncc(S(=N)(=O)C3CCN(c4ccc(C(=O)NC)nc4)CC3)cc2[nH]c1=O] |
| 2.8 | 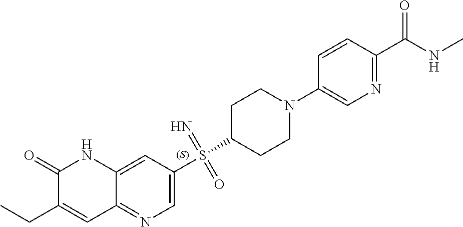 |
| 2.7-1 | 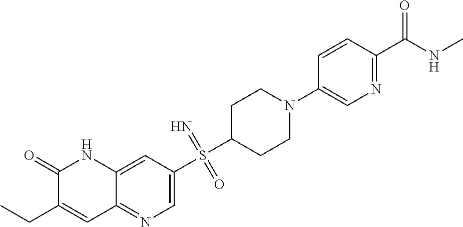 [SMILES: CCc1cc2ncc(S(=N)(=O)C3CCN(c4ccc(C(=O)NC)nc4)CC3)cc2[nH]c1=O] |

Although certain compounds described in Table 1 are presented as specific stereoisomers and/or in a non-stereochemical form, it is understood that any or all stereochemical forms, including any enantiomeric or diastereomeric forms, and any tautomers or other forms of any of the compounds of Table 1 are herein described. In some embodiments, the compound described herein is selected from Compound Nos. 2.1-2.8.

This disclosure also includes all salts, such as pharmaceutically acceptable salts, of compounds referred to herein. This disclosure also includes any or all of the stereochemical forms, including any enantiomeric or diastereomeric forms, and any tautomers or other forms, such as N-oxides, solvates, hydrates, or isotopomers, of the compounds described. The present disclosure also includes co-crystals of the compounds described herein. Unless stereochemistry is explicitly indicated in a chemical structure or name, the structure or name is intended to embrace all possible stereoisomers of a compound depicted. In addition, where a specific stereochemical form is depicted, it is understood that other stereochemical forms are also embraced by the invention. All forms of the compounds are also embraced by the invention, such as crystalline or non-crystalline forms of the compounds. Compositions comprising a compound of the invention are also intended, such as a composition of substantially pure compound, including a specific stereochemical form thereof. Compositions comprising a mixture of compounds of the invention in any ratio are also embraced by the invention, including mixtures of two or more stereochemical forms of a compound of the invention in any ratio, such that racemic, non-racemic, enantioenriched and scalemic mixtures of a compound are embraced.

In the descriptions herein, it is understood that every description, variation, embodiment, or aspect of a moiety can be combined with every description, variation, embodiment, or aspect of other moieties the same as if each and every combination of descriptions is specifically and individually listed. For example, every description, variation, embodiment, or aspect provided herein with respect to X¹ of formula (I) may be combined with every description, variation, embodiment, or aspect of X², X³, X⁴, X⁵, A, B, R¹, R², R³, R⁴, R⁵, R⁶, R⁷, E, m and/or n, the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae. For example, all descriptions, variations, embodiments, or aspects of formula (I), where applicable, apply equally to any of formulae (I), (I-1), (I-2), (I-A), (I-B), (I-C), (I-D), (I-E), (I-F), (I-G), (I-H), (I-1-a), (I-1-b), (I-1-c), (I-1-d), (I-1-e), (I-1-f), (I-2-a), (I-2-b), (I-2-c), (I-2-d), (I-2-e), and (I-2-f) detailed herein, and are equally described, the same as if each and every description, variation, embodiment or aspect were separately and individually listed for all formulae.

### III. General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

The intermediates described in the following preparations may contain a number of nitrogen, hydroxy, and acid protecting groups such as esters. The variable protecting group may be the same or different in each occurrence depending on the particular reaction conditions and the particular transformations to be performed. The protection and deprotection conditions are well known to the skilled artisan and are described in the literature. See e.g., Greene and Wuts, Protective Groups in Organic Synthesis, (T. Greene and P. Wuts, eds., 2d ed. 1991).

Certain stereochemical centers have been left unspecified and certain substituents have been eliminated in the following schemes for the sake of clarity and are not intended to limit the teaching of the schemes in any way. Furthermore, individual isomers, enantiomers, and diastereomers may be separated or resolved by one of ordinary skill in the art at any convenient point in the synthesis of compounds of the invention, by methods such as selective crystallization techniques or chiral chromatography (See e.g., J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

The compounds of the present invention, or a pharmaceutically acceptable salt, solvate, hydrate, or co-crystal thereof, or a mixture of any of the foregoing, may be prepared by a variety of procedures known in the art, some of which are illustrated in the Examples below. The specific synthetic steps for each of the routes described may be combined in different ways, to prepare compounds of the present disclosure, or salts thereof. The products of each step can be recovered by conventional methods well known in the art, including extraction, evaporation, precipitation, chromatography, filtration, trituration, and crystallization. The reagents and starting materials are readily available to one of ordinary skill in the art. Others may be made by standard techniques of organic and heterocyclic chemistry which are analogous to the syntheses of known structurally-similar compounds and the procedures described in the Examples which follow including any novel procedures.

Compounds of formula (I) can be prepared according to Scheme A, Scheme B, Scheme C, Scheme D, Scheme E, Scheme F, Scheme G, or Scheme H, wherein X¹, X², X³, X⁴, X⁵, A, B, R¹, R², R³, R⁴, R⁵, R⁶, R⁷, E, m and n are as defined for formulae (I), (I-1), (I-2), (I-1-a), (I-1-b), (I-1-c), (I-1-d), (I-1-e), (I-1-f), (I-2-a), (I-2-b), (I-2-c), (I-2-d), (I-2-e), or (I-2-f). or any applicable variation thereof as detailed herein.

Compounds of formula (I) may be prepared according to general synthetic scheme shown in Scheme A. In Scheme A, oxo-substituted N-heteroaromatic fused bicyclic compounds A-a are coupled with thiolated piperidinyl (B═CH) compounds A-b to provide intermediate thioether compounds A-c. The intermediate compounds A-c are treated with suitable reagents to induce oxidation (and imine formation, if applicable) on the thioether moiety to provide compounds of Formula (I). Schemes B and C illustrate general synthetic schemes for the compounds of Formula (I-1) and Formula (I-2).

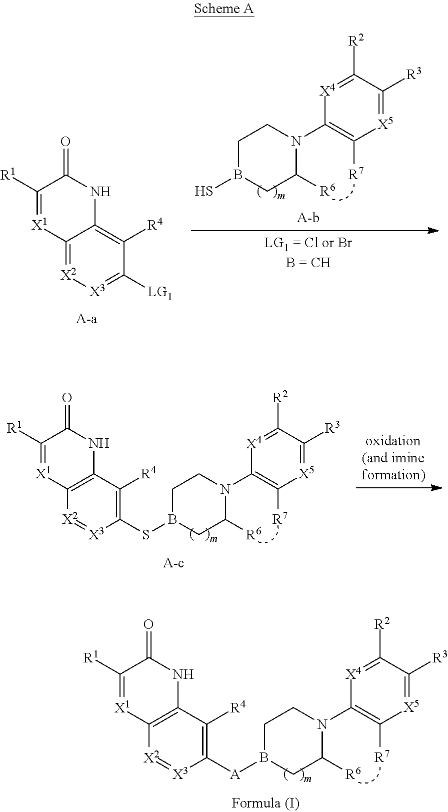

Compounds of formula (I-1) may be prepared according to general synthetic scheme shown in Scheme B. In Scheme B, oxo-substituted N-heteroaromatic fused bicyclic compounds A-a are treated with suitable thiolating reagents, such as benzyl mercaptan, to provide intermediate thioether compounds B-b. The thioether compounds B-b are subjected to further oxidation (and imine formation, if applicable), such as with thiocyanate or m-chloroperoxybenzoic acid and/or ammonium carbamate with (diacetoxyiodo)benzene, to provide sulfone or sulfoximine-modified compounds B-c. As illustrated in Scheme B, the sulfone or sulfoximine-modified compounds B-c are prepared such that a suitable leaving group is installed on the sulfur atom of the “A” moiety. The compounds B-c are subsequently coupled to fused N-heterocyclic compounds B-d to provide compounds of Formula (I-1).

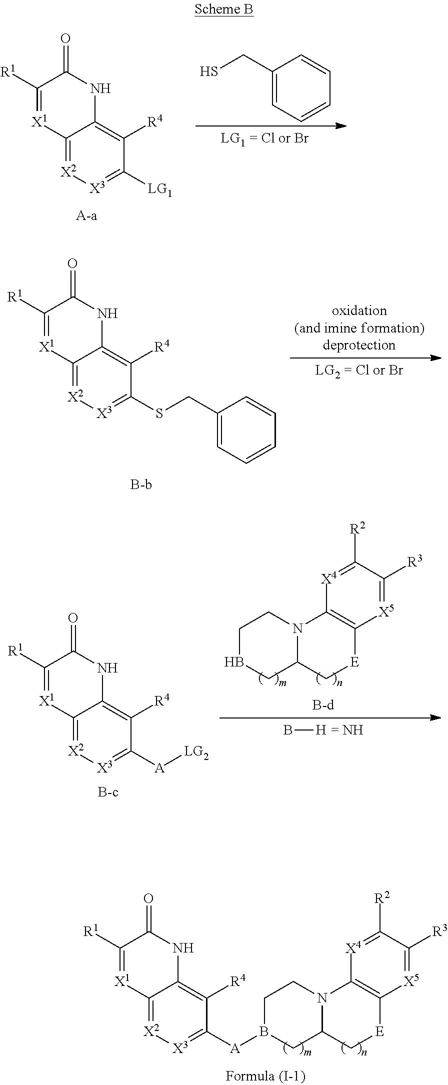

Compounds of formula (I-2) may be prepared according to general synthetic scheme shown in Scheme C. In Scheme C, oxo-substituted N-heteroaromatic fused bicyclic compounds A-a are treated with suitable thiolated piperidinyl (B═CH) compounds C-b to provide intermediate thioether compounds C-c. The nitrogen atom of the thiolated piperidinyl (B═CH) compounds C-b may be optionally protected by a suitable protecting group PG for an amine, such as a Boc protecting group. The thioether compounds C-c are subjected to further oxidation (and imine formation, if applicable) to provide sulfone or sulfoximine-modified compounds C-d. The sulfone or sulfoximine-modified compounds C-d may subsequently be deprotected to remove any protecting groups present to provide intermediate compounds C-e. The compounds C-e are then coupled with (hetero)aromatic rings C-f to provide compounds of Formula (I-2).

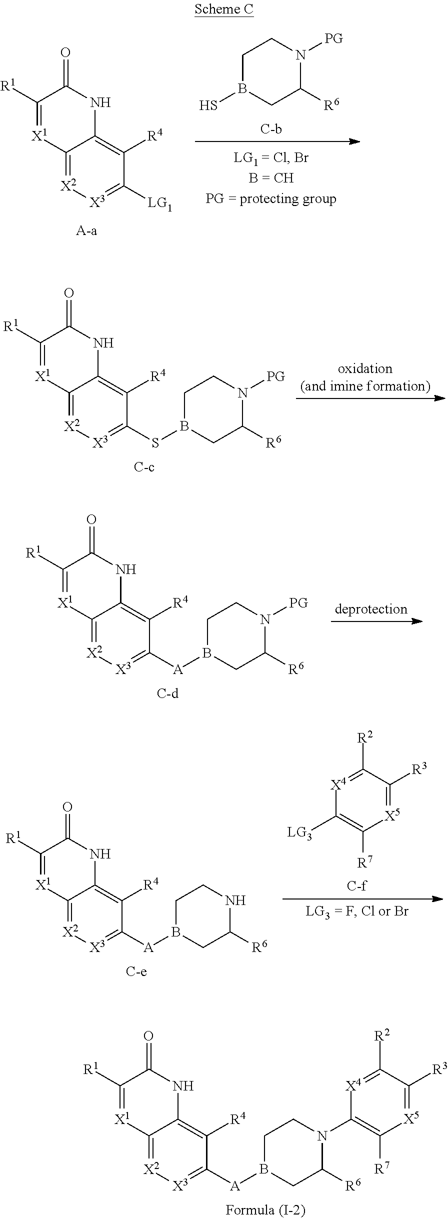

Schemes D-F illustrate exemplary synthetic schemes for preparing the compounds A-a. In Scheme D, an exemplary scheme for the preparation of intermediate compound A-a-1, wherein X¹ is CH, is shown. Schemes E-F illustrate exemplary schemes for the preparation of intermediate compound A-a-2, wherein X¹ is N.

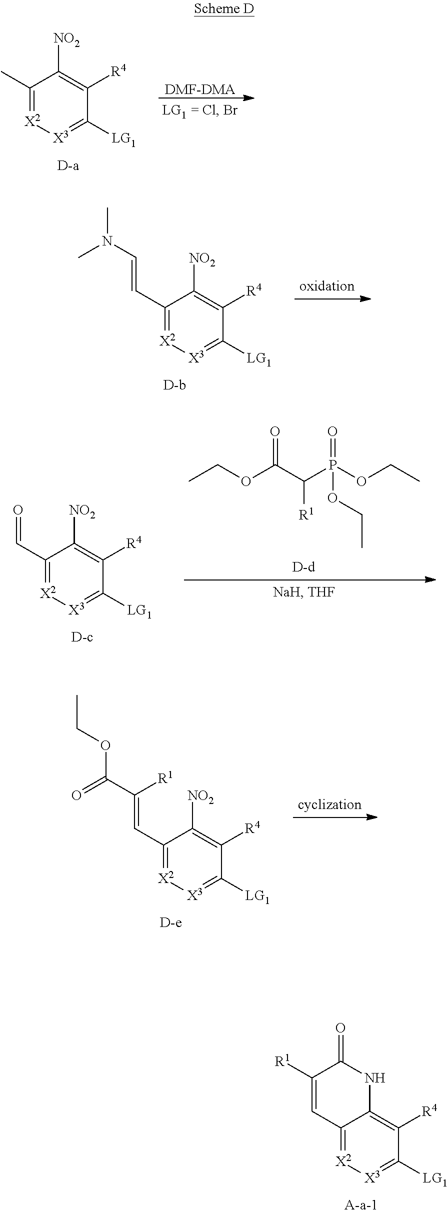

In Scheme D, nitrosylated (hetero)aromatic compounds D-a are treated with N,N-dimethylformamide dimethyl acetal (DMF-DMA) to provide intermediate compounds D-b. The intermediate compounds D-b are oxidized, such as with sodium (meta)periodate, to give formylated, nitrosylated intermediate compounds D-c. The intermediate compounds D-c are subsequently reacted with phosphonate esters D-d to provide the compounds D-e. The compounds D-e are reduced with suitable reducing agents, such as elemental iron with ammonium chloride, to enable cyclization to form oxo-substituted N-heteroaromatic fused bicyclic compounds A-a-1.

In Scheme E, nitrosylated (hetero)aromatic compounds E-a are coupled to amino-substituted esters E-b under suitable conditions, such as in the presence of a base, e.g., DIEA, to provide intermediate compounds E-c. The coupled intermediate compounds E-c are treated with suitable reducing agents, such as elemental iron with ammonium chloride, to enable cyclization to form the cyclized compounds E-d. The cyclized compounds E-d are oxidized, such as with DDQ or HBr, to provide oxo-substituted N-heteroaromatic fused bicyclic compounds A-a-2.

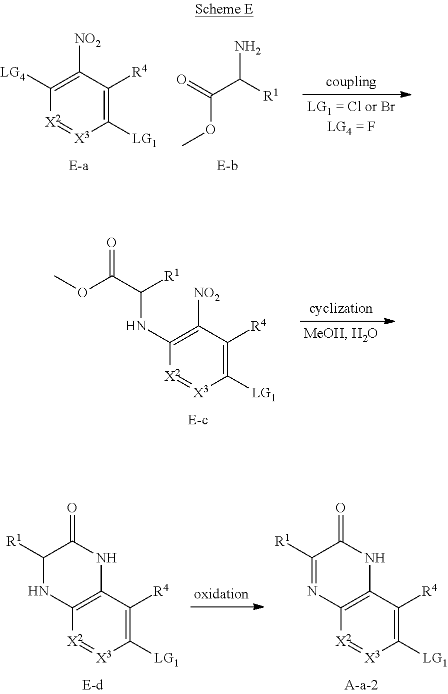

In Scheme F, as an alternative to Scheme E, aminated (hetero)aromatic compounds F-a are reacted with R¹-substituted methyl 2-oxopropanoate compounds F-b under suitable conditions, such as in the presence of acetic acid, to both couple the compounds F-a and F-b together and to cyclize the coupled reaction intermediate to directly provide the oxo-substituted N-heteroaromatic fused bicyclic compounds A-a-2.

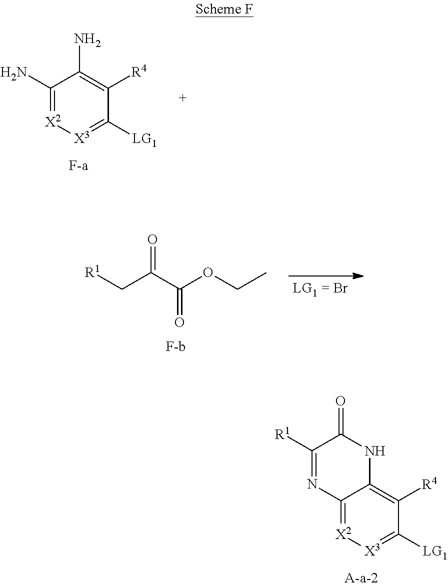

Scheme G below illustrates an exemplary synthetic scheme for the preparation of N-heterocyclic fused compounds B-d, as utilized in Scheme B above. In Scheme G, hydroxyalkyl-substituted piperidinyl or piperazinyl compounds G-a are cyclized in the presence of thionyl chloride (SOCl₂) to provide fused bicyclic compounds G-b. The fused bicyclic compounds G-b are oxidized, such as with sodium (meta)periodate, to give the corresponding sulfite compounds G-c. The intermediate compounds G-c are coupled with E-substituted (hetero)aromatic compounds G-d with suitable reagents, such as sodium hydride, to provide the compounds G-e. The compounds G-e are cyclized under suitable conditions, such as in the presence of Pd-PEPPSI-IPentCl, to give the protected tricyclic compounds G-f, which may be subsequently deprotected to provide N-heterocyclic fused compounds B-d.

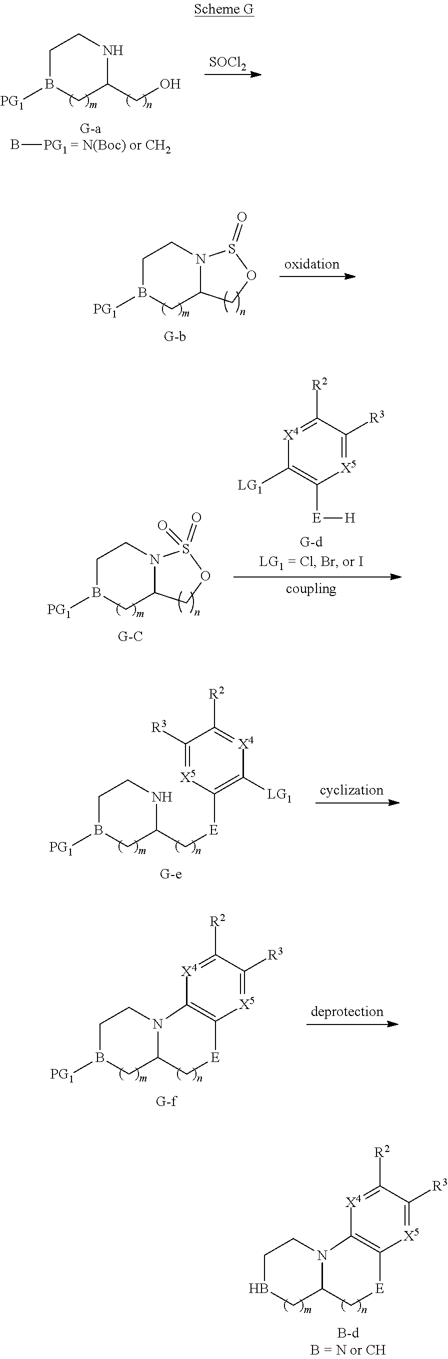

In Scheme H, an exemplary synthesis for an intermediate compound H-d is illustrated. Starting (hetero)aromatic compounds H-a are coupled with protected piperidinyl (B ═CH) or piperazinyl (B═N) compounds H-b to give the coupled intermediate compounds H-c. The compounds H-c are deprotected to provide the corresponding free intermediate compounds H-d.

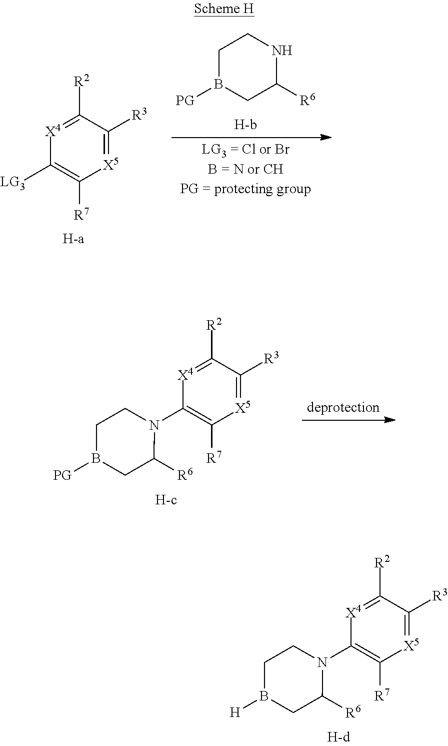

It should be recognize that the ring cyclization, coupling, oxidation/imine formation and deprotection can be carried out in one or more different orders than described herein.

It should be recognized that the present disclosure also provides for any intermediates of the compounds and methods for synthesizing the compounds as described herein. In another aspect, provided herein are general intermediates as described in any one of Schemes A through H above, or compound-specific intermediates as described in the examples below. It should be further recognized that the present disclosure also provides for synthetic methods comprising any individual step or combination of individual process steps, or compositions of synthetic intermediates and/or reaction products as described herein.

### IV. Pharmaceutical Compositions and Formulations

Any of the compounds described herein may be formulated as a pharmaceutically acceptable composition.

Pharmaceutical compositions of any of the compounds detailed herein are embraced by this disclosure. Thus, the present disclosure includes pharmaceutical compositions comprising a compound as detailed herein, or a pharmaceutically acceptable salt, solvate, hydrate, or co-crystal thereof, or a mixture of any of the foregoing, and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound, or a pharmaceutically acceptable salt, solvate, hydrate, or co-crystal thereof, or a mixture of any of the foregoing, as detailed herein are provided, such as compositions of substantially pure compounds. In some embodiments, a composition containing a compound, or a pharmaceutically acceptable salt, solvate, hydrate, or co-crystal thereof, or a mixture of any of the foregoing, as detailed herein is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound selected from a compound of Table 1 intends a composition that contains no more than 35% impurity, wherein the impurity denotes a compound other than the compound of Table 1. In one variation, a composition of substantially pure compound, or a pharmaceutically acceptable salt, solvate, hydrate, or co-crystal thereof, or a mixture of any of the foregoing, is provided wherein the composition contains no more than 25% impurity. In another variation, a composition of substantially pure compound, or a pharmaceutically acceptable salt, solvate, hydrate, or co-crystal thereof, or a mixture of any of the foregoing, is provided wherein the composition contains or no more than 20% impurity. In still another variation, a composition of substantially pure compound, or a pharmaceutically acceptable salt, solvate, hydrate, or co-crystal thereof, or a mixture of any of the foregoing, is provided wherein the composition contains or no more than 10% impurity. In a further variation, a composition of substantially pure compound, or a pharmaceutically acceptable salt, solvate, hydrate, or co-crystal thereof, or a mixture of any of the foregoing, is provided wherein the composition contains no more than 5% impurity. In another variation, a composition of substantially pure compound, or a pharmaceutically acceptable salt, solvate, hydrate, or co-crystal thereof, or a mixture of any of the foregoing, is provided wherein the composition contains no more than 3% impurity. In still another variation, a composition of substantially pure compound, or a pharmaceutically acceptable salt, solvate, hydrate, or co-crystal thereof, or a mixture of any of the foregoing, is provided wherein the composition contains no more than 1% impurity. In a further variation, a composition of substantially pure compound, or a pharmaceutically acceptable salt, solvate, hydrate, or co-crystal thereof, or a mixture of any of the foregoing, is provided wherein the composition contains no more than 0.5% impurity. In yet other variations, a composition of substantially pure compound means that the composition contains no more than 15%, no more than 10%, no more than 5%, no more than 3%, or no more than 1% impurity, which impurity may be the compound in a different stereochemical form. For instance, and without limitation, a composition of substantially pure (S) compound means that the composition contains no more than 15% or no more than 10% or no more than 5% or no more than 3% or no more than 1% of the (R) form of the compound.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein. In some embodiments, the compounds and compositions as provided herein are sterile. Methods for sterilization known in the art may be suitable for any compounds or form thereof and compositions thereof as detailed herein.

A compound detailed herein, or a pharmaceutically acceptable salt, solvate, hydrate, or co-crystal thereof, or a mixture of any of the foregoing, may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound, or a pharmaceutically acceptable salt, solvate, hydrate, or co-crystal thereof, or a mixture of any of the foregoing, may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

A compound detailed herein, or a pharmaceutically acceptable salt, solvate, hydrate, or co-crystal thereof, or a mixture of any of the foregoing, can be used in the preparation of a formulation, such as a pharmaceutical formulation, by combining the compound or compounds, or a pharmaceutically acceptable salt, solvate, hydrate, or co-crystal thereof, or a mixture of any of the foregoing, with a pharmaceutically acceptable carrier. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, PA, 20th ed. (2000), which is incorporated herein by reference.

A compound detailed herein, or a pharmaceutically acceptable salt, solvate, hydrate, or co-crystal thereof, or a mixture of any of the foregoing, may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds, or a pharmaceutically acceptable salt, solvate, hydrate, or co-crystal thereof, or a mixture of any of the foregoing, described herein can be formulated in a tablet in any dosage form described, for example, a compound as described herein, or a pharmaceutically acceptable salt, solvate, hydrate, or co-crystal thereof, or a mixture of any of the foregoing, can be formulated as a 10 mg tablet.

Compositions comprising a compound, or a pharmaceutically acceptable salt, solvate, hydrate, or co-crystal thereof, or a mixture of any of the foregoing, provided herein are also described. In one variation, the composition comprises a compound, or a pharmaceutically acceptable salt, solvate, hydrate, or co-crystal thereof, or a mixture of any of the foregoing, and a pharmaceutically acceptable carrier or excipient. In another variation, a composition of substantially pure compound, or a pharmaceutically acceptable salt, solvate, hydrate, or co-crystal thereof, or a mixture of any of the foregoing, is provided. In some embodiments, the composition is for use as a human or veterinary medicament. In some embodiments, the composition is for use in a method described herein. In some embodiments, the composition is for use in the treatment of a disease or disorder described herein.

Compositions formulated for co-administration of a compound provided herein and one or more additional pharmaceutical agents are also described. The co-administration can be simultaneous or sequential in any order. A compound provided herein may be formulated for co-administration with the one or more additional pharmaceutical agents in the same dosage form (e.g., single tablet or single i.v.) or separate dosage forms (e.g., two separate tablets, two separate i.v., or one tablet and one i.v.). Furthermore, co-administration can be, for example, 1) concurrent delivery, through the same route of delivery (e.g., tablet or i.v.), 2) sequential delivery on the same day, through the same route or different routes of delivery, or 3) delivery on different days, through the same route or different routes of delivery.

### V. Methods of Use

Compounds and compositions detailed herein, such as a pharmaceutical composition containing a compound of formula (I) or any variation thereof provided herein, or a pharmaceutically acceptable salt, solvate, hydrate, or co-crystal thereof, or a mixture of any of the foregoing, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In one aspect, provided herein are methods of a method of treating a cancer or neoplastic disease in a human in need thereof.

In some embodiments, the cancer or neoplastic disease is associated with one or more mutations in the DNA damage response activation. In certain embodiments, the one or more mutations in the DNA damage response activation is a mutation of ATM, ATR, BAP1, BARD1, BLM, BRCA1, BRCA2, BRIP1, CDK12, CHEK1, CHEK2, CCNE1 amplification, MRE11A, NBN, PALB2, PP2R²ARAD50, RAD51, RAD51B, RAD51C, RAD51D, RAD54L, XRCC2 and the FANC family of proteins.

In some embodiments, the cancer or neoplastic disease is associated with one or more mutations in a DNA damage repair enzyme. In certain embodiments, the repair enzyme assists in the repair of single-stranded lesions including breaks, nicks, base, or nucleotide modifications (via base-excision repair (BER), nucleotide excision repair (NER) or mismatch repair (MMR)) and double-stranded breaks (via either homologous recombination (HR) or the more common non-homologous end-joining (NHEJ)).

In some embodiments, the cancer or neoplastic disease is pancreatic, prostate, bladder, endometrial, lung, colorectal, ovarian, peritoneal, or breast cancer.

In some embodiments, the method further comprises administering one or more additional pharmaceutical agents. In some embodiments, the method further comprises administering one or more pharmaceutical agents that induces DNA damage and/or replicative stress, inhibits a protein or enzyme that regulates DDR or DNA replication, or induces anti-tumor immune response, or any combination(s) thereof. In certain embodiments, the one or more pharmaceutical agents that induces DNA damage and/or replicative stress is an alkylating agent (e.g., temozolimide, nitrosoureas, mitomycin C), platinum-based anti-neoplastic agent (e.g., cisplatin, oxaliplatin, carboplatin), anti-metabolite (e.g., 5-fluorouracil (5-FU), gemcitabine, fludarabine, methotrexate), topoisomerase inhibitor (e.g., etoposide, irinotecan), ionizing radiation, radiomimetic, or other radiotherapy. In certain other embodiments, the one or more pharmaceutical agents that inhibits a protein or enzyme that regulates DDR or DNA replication is an inhibitor of ATR, ATM, DNA-PK, CHK1, CHK2, WEE1, MYT1, polθ, EGFR, ErbB2, MET, FGFR1-4, VEGFR2, MEK, ERK, PI3K, AKT, mTORC1, or mTORC2. In still certain other embodiments, the one or more pharmaceutical agents that induces anti-tumor immune response is anti-PD1 therapy, anti-PD-L1 therapy, anti-CTLA4 therapy, cancer vaccine, T-cell-based therapy, T-cell engager bi-specific antibody, or oncolytic virus.

### VI. Dosing and Method of Administration

The dose of a compound described herein, or a pharmaceutically acceptable salt, solvate, hydrate, or co-crystal thereof, or a mixture of any of the foregoing, administered to an individual (such as a human) may vary with the particular compound or salt thereof, the method of administration, and the particular cancer, such as type and stage of cancer, being treated. In some embodiments, the amount of the compound, or a pharmaceutically acceptable salt, solvate, hydrate, or co-crystal thereof, or a mixture of any of the foregoing, is a therapeutically effective amount.

The compounds provided herein, or a pharmaceutically acceptable salt, solvate, hydrate, or co-crystal thereof, or a mixture of any of the foregoing, may be administered to an individual via various routes, including, e.g., intravenous, intramuscular, subcutaneous, oral, and transdermal.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein, or a pharmaceutically acceptable salt, solvate, hydrate, or co-crystal thereof, or a mixture of any of the foregoing, and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

In some embodiments, the individual is a mammal. In some embodiments, the individual is a primate, dog, cat, rabbit, or rodent. In some embodiments, the individual is a primate. In some embodiments, the individual is a human. In some embodiments, the human is at least about or is about any of 18, 21, 30, 50, 60, 65, 70, 75, 80, or 85 years old. In some embodiments, the human is a child. In some embodiments, the human is less than about or about any of 21, 18, 15, 10, 5, 4, 3, 2, or 1 years old.

### VII. Articles of Manufacture and Kits

The present disclosure further provides articles of manufacture comprising a compound described herein, or a pharmaceutically acceptable salt, solvate, hydrate, or co-crystal thereof, or a mixture of any of the foregoing, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein, or a pharmaceutically acceptable salt, solvate, hydrate, or co-crystal thereof, or a mixture of any of the foregoing, thereof. The kits may be used for any one or more of the uses described herein, and, accordingly, may contain instructions for the treatment of any disease or described herein, for example for the treatment of cancer or neoplastic disease, such as those associated with one or more mutations in the DNA damage response activation, or those associated with one or more mutations in a DNA damage repair enzyme.

In certain embodiments, the one or more mutations in the DNA damage response activation is a mutation of ATM, ATR, BAP1, BARD1, BLM, BRCA1, BRCA2, BRIP1, CDK12, CHEK1, CHEK2, CCNE1 amplification, MRE11A, NBN, PALB2, PP2R²ARAD50, RAD51, RAD51B, RAD51C, RAD51D, RAD54L, XRCC2 and the FANC family of proteins. In certain embodiments, the repair enzyme assists in the repair of single-stranded lesions including breaks, nicks, base, or nucleotide modifications (via base-excision repair (BER), nucleotide excision repair (NER) or mismatch repair (MMR)) and double-stranded breaks (via either homologous recombination (HR) or the more common non-homologous end-joining (NHEJ)).

The kits may be used for any one or more of the uses described herein, and, accordingly, may contain instructions for the treatment of any disease or described herein, for example for the treatment of cancer or neoplastic disease, for example, wherein the cancer or neoplastic disease is pancreatic, prostate, bladder, endometrial, lung, colorectal, ovarian, peritoneal, or breast cancer.

The kits optionally further comprise a container comprising one or more additional pharmaceutical agents and which kits further comprise instructions on or in the package insert for treating the subject with an effective amount of the one or more additional pharmaceutical agents.

Kits generally comprise suitable packaging. The kits may comprise one or more containers comprising any compound described herein. Each component (if there is more than one component) can be packaged in separate containers or some components can be combined in one container where cross-reactivity and shelf-life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### ENUMERATED EMBODIMENTS

The following enumerated embodiments are representative of some aspects of the invention.
1. A compound of formula (I)

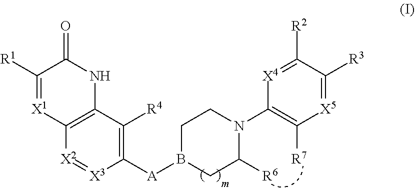

or a pharmaceutically acceptable salt, solvate, hydrate, or co-crystal thereof, or a mixture of any of the foregoing, wherein:
   X¹, X² and X³ are each independently N or C—R¹;
   X⁴ and X⁵ are each independently N or CH;
   A is —S(═O)₂—, —S(═O)(═NH)— or —S(═O)(═N—CH₃)—;
   B is N or CH;
   each R¹ is independently H, halogen, C₁-C₄ alkyl, C₁-C₄ fluoroalkyl, C₁-C₄ alkoxy, or C₃-C₆ cycloalkyl;
   R² is H, halogen, C₁-C₄ alkyl, or C₁-C₄ fluoroalkyl;
   R³ is halogen or —C(═O)NH—R⁵;
   R⁴ is H, halogen, C₁-C₄ alkyl, or C₁-C₄ fluoroalkyl; and
   each R⁵ is independently H, CD₃, C₁-C₄ alkyl, C₁-C₄ fluoroalkyl, C₁₋₄ alkoxy, C₃-C₆ cycloalkyl, or 4- to 7-membered heterocycloalkyl;
   R⁶ is H; and
   R⁷ is H, halogen, CN, C₁-C₄ alkyl, C₁-C₄ fluoroalkyl, C₁-C₄ alkoxy, or C₃-C₆ cycloalkyl; or
   R⁶ and R⁷ are taken together with the atoms to which they are attached to form a ring, wherein R⁶ is —(CH₂)ₙ— and R⁷ is E, wherein E is —O—, —S—, —NR⁵—, or —CR⁵—;
   m is an integer of 1 or 2, wherein when R⁶ is H, then m is 1; and
   n is an integer of 1 or 2.
2. The compound of embodiment 1, or a pharmaceutically acceptable salt, solvate, hydrate, or co-crystal thereof, or a mixture of any of the foregoing, wherein the compound is a compound of formula (I-1)

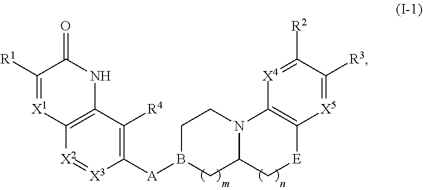

wherein:
E is —O—, —S—, —NR⁵—, or —CR⁵—;
m is an integer of 1 or 2; and
n is an integer of 1 or 2.
3. The compound of embodiment 1, or a pharmaceutically acceptable salt, solvate, hydrate, or co-crystal thereof, or a mixture of any of the foregoing, wherein the compound is a compound of formula (I-2)

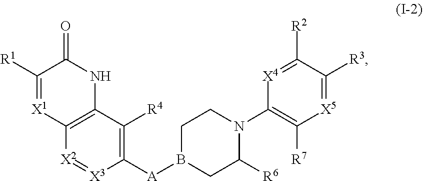

wherein:
R⁶ is H; and
R⁷ is H, halogen, CN, C₁-C₄ alkyl, C₁-C₄ fluoroalkyl, C₁-C₄ alkoxy, or C₃-C₆ cycloalkyl.
4. The compound of any one of embodiments 1 to 3, or a pharmaceutically acceptable salt, solvate, hydrate, or co-crystal thereof, or a mixture of any of the foregoing, wherein A is —S(O)₂—.
5. The compound of any one of embodiments 1 to 3, or a pharmaceutically acceptable salt, solvate, hydrate, or co-crystal thereof, or a mixture of any of the foregoing, wherein A is —S(O)(═NH)—.
6. The compound of any one of embodiments 1 to 3, or a pharmaceutically acceptable salt, solvate, hydrate, or co-crystal thereof, or a mixture of any of the foregoing, wherein A is —S(O)(═N—CH₃)—.
7. The compound of any one of embodiments 1 to 6, or a pharmaceutically acceptable salt, solvate, hydrate, or co-crystal thereof, or a mixture of any of the foregoing, wherein B is N.
8. The compound of any one of embodiments 1 to 6, or a pharmaceutically acceptable salt, solvate, hydrate, or co-crystal thereof, or a mixture of any of the foregoing, wherein B is CH.
9. The compound of embodiment 1 or 2, or a pharmaceutically acceptable salt, solvate, hydrate, or co-crystal thereof, or a mixture of any of the foregoing, wherein the compound is a compound of formula (I-1-a), (I-1-b), (I-1-c), (I-1-d), (I-1-e), or (I-1-f):

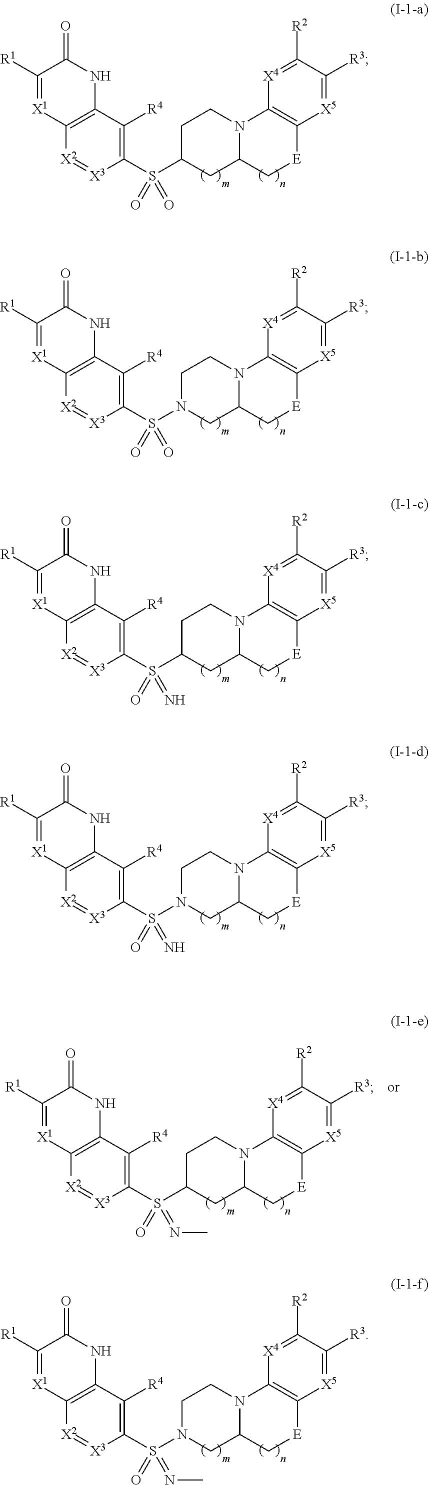

10. The compound of embodiment 1 or 3, or a pharmaceutically acceptable salt, solvate, hydrate, or co-crystal thereof, or a mixture of any of the foregoing, wherein the compound is a compound of formula (I-2-a), (I-2-b), (I-2-c), (I-2-d), (I-2-e), or (I-2-f):

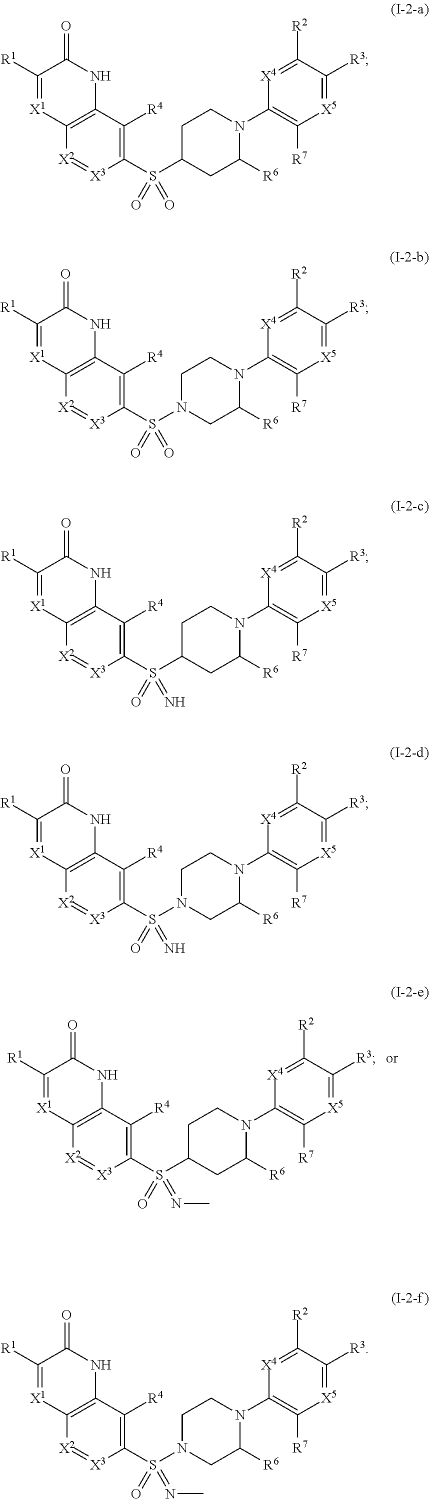

11. The compound of any one of embodiments 1 to 10, or a pharmaceutically acceptable salt, solvate, hydrate, or co-crystal thereof, or a mixture of any of the foregoing, wherein X¹ is N.
12. The compound of any one of embodiments 1 to 10, or a pharmaceutically acceptable salt, solvate, hydrate, or co-crystal thereof, or a mixture of any of the foregoing, wherein X¹ is CH.
13. The compound of any one of embodiments 1 to 12, or a pharmaceutically acceptable salt, solvate, hydrate, or co-crystal thereof, or a mixture of any of the foregoing, wherein X² is N.
14. The compound of any one of embodiments 1 to 12, or a pharmaceutically acceptable salt, solvate, hydrate, or co-crystal thereof, or a mixture of any of the foregoing, wherein X² is CH.
15. The compound of any one of embodiments 1 to 10, or a pharmaceutically acceptable salt, solvate, hydrate, or co-crystal thereof, or a mixture of any of the foregoing, wherein one of X¹ and X² is N and the other of X¹ and X² is CH.
16. The compound of any one of embodiments 1 to 15, or a pharmaceutically acceptable salt, solvate, hydrate, or co-crystal thereof, or a mixture of any of the foregoing, wherein X³ is N.
17. The compound of any one of embodiments 1 to 15, or a pharmaceutically acceptable salt, solvate, hydrate, or co-crystal thereof, or a mixture of any of the foregoing, wherein X³ is CH or CF.
18. The compound of any one of embodiments 1 to 17, or a pharmaceutically acceptable salt, solvate, hydrate, or co-crystal thereof, or a mixture of any of the foregoing, wherein X⁴ is N.
19. The compound of any one of embodiments 1 to 17, or a pharmaceutically acceptable salt, solvate, hydrate, or co-crystal thereof, or a mixture of any of the foregoing, wherein X⁴ is CH.
20. The compound of any one of embodiments 1 to 19, or a pharmaceutically acceptable salt, solvate, hydrate, or co-crystal thereof, or a mixture of any of the foregoing, wherein X⁵ is N.
21. The compound of any one of embodiments 1 to 19, or a pharmaceutically acceptable salt, solvate, hydrate, or co-crystal thereof, or a mixture of any of the foregoing, wherein X⁵ is CH.
22. The compound of any one of embodiments 1 to 10 and 15, or a pharmaceutically acceptable salt, solvate, hydrate, or co-crystal thereof, or a mixture of any of the foregoing, wherein
one of X¹ and X² is N and the other of X¹ and X² is CH;
X³ is CH or CF;
X⁴ is CH; and
X⁵ is N or CH.
23. The compound of any one of embodiments 1 to 22, or a pharmaceutically acceptable salt, solvate, hydrate, or co-crystal thereof, or a mixture of any of the foregoing, wherein R¹ is H or C₁-C₄ alkyl.
24. The compound of embodiment 23, or a pharmaceutically acceptable salt, solvate, hydrate, or co-crystal thereof, or a mixture of any of the foregoing, wherein R¹ is H, —CH₃ or —CH₂CH₃.
25. The compound of any one of embodiments 1 to 24, or a pharmaceutically acceptable salt, solvate, hydrate, or co-crystal thereof, or a mixture of any of the foregoing, wherein R² is H or halogen.
26. The compound of any one of embodiments 1 to 24, or a pharmaceutically acceptable salt, solvate, hydrate, or co-crystal thereof, or a mixture of any of the foregoing, wherein R² is C₁-C₄ alkyl or C₁-C₄ fluoroalkyl.
27. The compound of any one of embodiments 1 to 26, or a pharmaceutically acceptable salt, solvate, hydrate, or co-crystal thereof, or a mixture of any of the foregoing, wherein R³ is halogen.
28. The compound of embodiment 27, or a pharmaceutically acceptable salt, solvate, hydrate, or co-crystal thereof, or a mixture of any of the foregoing, wherein R³ is F or Cl.
29. The compound of any one of embodiments 1 to 28, or a pharmaceutically acceptable salt, solvate, hydrate, or co-crystal thereof, or a mixture of any of the foregoing, wherein R³ is —C(═O)NH—R⁵.
30. The compound of embodiment 29, or a pharmaceutically acceptable salt, solvate, hydrate, or co-crystal thereof, or a mixture of any of the foregoing, wherein R⁵ is C₁-C₄ alkyl.
31. The compound of embodiment 30, or a pharmaceutically acceptable salt, solvate, hydrate, or co-crystal thereof, or a mixture of any of the foregoing, wherein R⁵ is —CH₃.
32. The compound of any one of embodiments 1 to 31, or a pharmaceutically acceptable salt, solvate, hydrate, or co-crystal thereof, or a mixture of any of the foregoing, wherein R⁴ is H or halogen.
33. The compound of any one of embodiments 1 to 31, or a pharmaceutically acceptable salt, solvate, hydrate, or co-crystal thereof, or a mixture of any of the foregoing, wherein R⁴ is C₁-C₄ alkyl or C₁-C₄ fluoroalkyl.
34. The compound of any one of embodiments 1 to 33, or a pharmaceutically acceptable salt, solvate, hydrate, or co-crystal thereof, or a mixture of any of the foregoing, wherein R⁶ is H and R⁷ is H.
35. The compound of any one of embodiments 1 to 33, or a pharmaceutically acceptable salt, solvate, hydrate, or co-crystal thereof, or a mixture of any of the foregoing, wherein R⁶ is H and R⁷ is halogen.
36. The compound of embodiment 35, or a pharmaceutically acceptable salt, solvate, hydrate, or co-crystal thereof, or a mixture of any of the foregoing, wherein R⁷ is F.
37. A compound, wherein the compound is selected from the group consisting of:

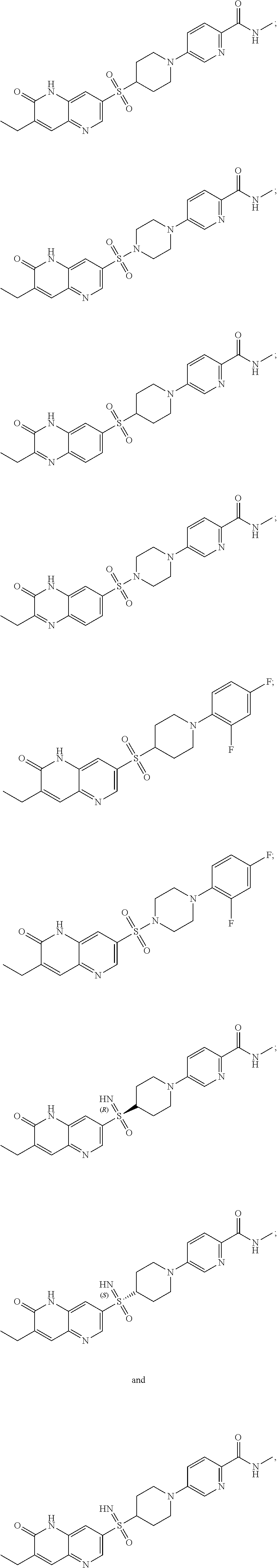

or a pharmaceutically acceptable salt, solvate, hydrate, or co-crystal thereof, or a mixture of any of the foregoing.

38. A pharmaceutical composition comprising the compound of any one of embodiments 1-37, or a pharmaceutically acceptable salt, solvate, hydrate, or co-crystal thereof, or a mixture of any of the foregoing, and one or more pharmaceutically acceptable excipients.

39. A method of inhibiting PARP1 enzymatic activity in a cell, comprising exposing the cell with an effective amount of a compound of any one of embodiments 1-37, or a pharmaceutically acceptable salt, solvate, hydrate, or co-crystal thereof, or a mixture of any of the foregoing, or a pharmaceutical composition according to embodiment 38.

40. The method of embodiment 39, wherein the cell is a cancer cell.

41. A method of treating a cancer or neoplastic disease in a human in need thereof, comprising administering to the human a compound of any one of embodiments 1-37, or a pharmaceutically acceptable salt, solvate, hydrate, or co-crystal thereof, or a mixture of any of the foregoing, or a pharmaceutical composition according to embodiment 38.
42. The method of embodiment 41, wherein the cancer or neoplastic disease is associated with one or more mutations in the DNA damage response activation.
43. The method of embodiment 42, wherein the one or more mutations in the DNA damage response activation is a mutation of ATM, ATR, BAP1, BARD1, BLM, BRCA1, BRCA2, BRIP1, CDK12, CHEK1, CHEK2, CCNE1 amplification, MRE11A, NBN, PALB2, PP2R2ARAD50, RAD51, RAD51B, RAD51C, RAD51D, RAD54L, XRCC2 and the FANC family of proteins.
44. The method of embodiment 41, wherein the cancer or neoplastic disease is associated with one or more mutations in a DNA damage repair enzyme.
45. The method of embodiment 44, wherein the repair enzyme assists in the repair of single-stranded lesions including breaks, nicks, base, or nucleotide modifications (via base-excision repair (BER), nucleotide excision repair (NER) or mismatch repair (MMR)) and double-stranded breaks (via either homologous recombination (HR) or the more common non-homologous end-joining (NHEJ)).
46. The method of any one of embodiments 41 to 45, wherein the cancer or neoplastic disease is pancreatic, prostate, bladder, endometrial, lung, colorectal, ovarian, peritoneal, or breast cancer.

### EXAMPLES

It is understood that the present disclosure has been made only by way of example, and that numerous changes in the combination and arrangement of parts can be resorted to by those skilled in the art without departing from the spirit and scope of present disclosure.

### Synthetic Examples

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

Abbreviations used in the Examples include the following: AcOH: acetic acid; ACN: acetonitrile; DCM: dichloromethane; DDQ: 2,3-Dichloro-5,6-dicyano-1,4-benzoquinone DMF: dimethylformamide; DMF-DMA: N,N-Dimethyl formamide dimethyl acetal, or 1,1-dimethoxy-N, N-dimethylmethanamine; DIEA: diisopropylethylamine; DMSO: dimethyl sulfoxide; Ephos: Dicyclohexyl(3-isopropoxy-2′,4′,6′-triisopropyl-[1,1′-biphenyl]-2-yl)phosphane; ¹H NMR: proton nuclear magnetic resonance; LCMS: liquid chromatography-mass spectrometry; m-CPBA: meta-chloroperoxybenzoic acid; MeOH: methanol or methyl alcohol; NCS: N-chlorosuccinimide; AcO or OAc: acetate; TEA: triethylamine; TFA: trifluoroacetic acid; THF: tetrahydrofuran; and TLC: thin-layer chromatography.

### Example 1. Synthesis of 5-[4-(7-ethyl-6-oxo-5H-1,5-naphthyridin-3-ylsulfonyl)piperidin-1-yl]-N-methylpyridine-2-carboxamide (Compound 2.1)

### Step 1. Synthesis of [(E)-2-(5-bromo-3-nitropyridin-2-yl)ethenyl]dimethylamine

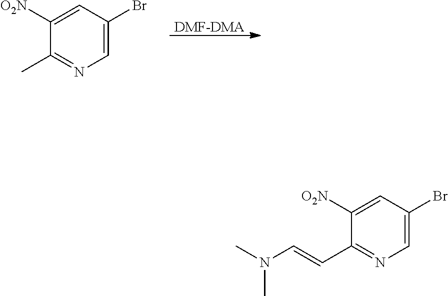

To a solution of 5-bromo-2-methyl-3-nitropyridine (10.0 g, 46.07 mmol) in DMF (250.0 mL) was added DMF-DMA (16.4 g, 138.23 mmol) at room temperature. The resulting mixture was stirred at 90° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford [(E)-2-(5-bromo-3-nitropyridin-2-yl)ethenyl]dimethylamine (10.0 g, 79%) as a red solid. LCMS (ESI, m/z): [M+H]⁺=272.0.

### Step 2. Synthesis of 5-bromo-3-nitropyridine-2-carbaldehyde

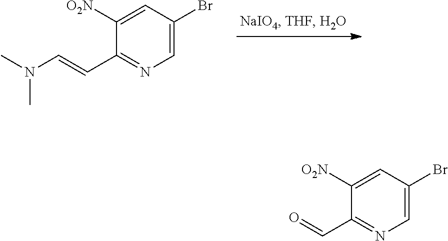

To a solution of [(E)-2-(5-bromo-3-nitropyridin-2-yl)ethenyl]dimethylamine (5.0 g, 6.01 mmol) in THF (50.0 mL)/H₂O (50.0 mL) was added NaIO₄ (11.7 g, 55.12 mmol) at room temperature. The resulting mixture was stirred at room temperature for 2 h. After the reaction was completed, the resulting mixture was concentrated under reduced pressure. The residue was purified by flash column chromatography with petroleum ether/ethyl acetate (1/1, v/v) to afford 5-bromo-3-nitropyridine-2-carbaldehyde (3.3 g, 77%) as a yellow solid. LCMS (ESI, m/z): [M+H]⁺=230.9.

### Step 3. Synthesis of ethyl (Z)-2-((5-bromo-3-nitropyridin-2-yl)methylene)butanoate

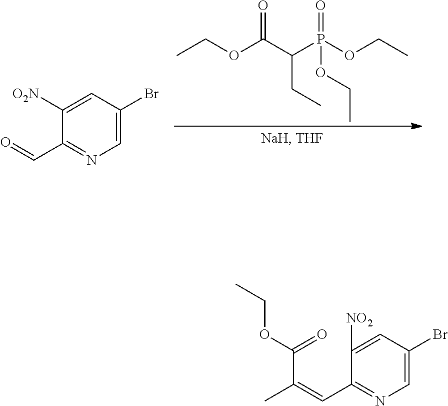

To a mixture of ethyl 2-(diethoxyphosphoryl)butanoate (7.3 g, 29.0 mmol) in THF (100.0 mL) was added NaH (1.2 g, 60%) at 0° C. under N₂. The mixture was stirred at room temperature for 1 h. Then 5-bromo-3-nitropyridine-2-carbaldehyde (2.8 g, 12.12 mmol) was added to the mixture at −78° C. The mixture was stirred at −78° C. for additional 10 min. After the reaction was completed, the reaction mixture was quenched with H₂O and then extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford ethyl (Z)-2-((5-bromo-3-nitropyridin-2-yl)methylene)butanoate (1.6 g, 40%) as a yellow oil. LCMS (ESI, m/z): [M+H]⁺=329.0.

### Step 4. Synthesis of 7-bromo-3-ethyl-1H-1,5-naphthyridin-2-one

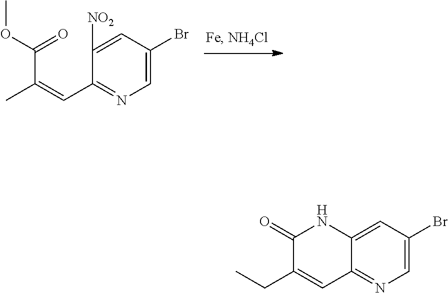 [SMILES: COC(=O)/C(C)=C\c1ncc(Br)cc1[N+](=O)[O-] ; CCc1cc2ncc(Br)cc2[nH]c1=O]

To a solution of ethyl (Z)-3-(5-bromo-3-nitropyridin-2-yl)-2-methylacrylate (1.5 g, 4.55 mmol) in AcOH (30.0 mL)/H₂O (7.5 mL) was added Fe (0.7 g, 13.67 mmol) at room temperature. The resulting mixture was stirred at 80° C. for 2 h. After the reaction was completed, the reaction mixture was filtered. The filtrate was evaporated under vacuo to afford 7-bromo-3-ethyl-1H-1,5-naphthyridin-2-one (1.5 g, crude) as a yellow solid. LCMS (ESI, m/z): [M+H]⁺=253.0.

### Step 5. Synthesis of tert-butyl 4-[(7-ethyl-6-oxo-5H-1,5-naphthyridin-3-yl)sulfanyl]piperidine-1-carboxylate

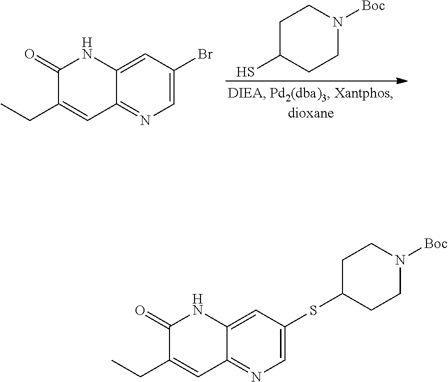 [SMILES: CCc1cc2ncc(Br)cc2[nH]c1=O ; CC(C)(C)OC(=O)N1CCC(S)CC1 ; CCc1cc2ncc(SC3CCN(C(=O)OC(C)(C)C)CC3)cc2[nH]c1=O]

To a mixture of 7-bromo-3-ethyl-1H-1,5-naphthyridin-2-one (1.0 g, 3.95 mmol) in dioxane (30.0 mL) was added tert-butyl 4-sulfanylpiperidine-1-carboxylate (1.7 g, 7.90 mmol), DIEA (1.5 g, 11.85 mmol), XantPhos (457.2 mg, 0.79 mmol) and Pd₂(dba)₃ (361.8 mg, 0.39 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford tert-butyl 4-[(7-ethyl-6-oxo-5H-1,5-naphthyridin-3-yl)sulfanyl]piperidine-1-carboxylate (600.0 mg, 38%) as a yellow solid. LCMS (ESI, m/z): [M+H]⁺=390.2.

### Step 6. Synthesis of tert-butyl 4-(7-ethyl-6-oxo-5H-1,5-naphthyridin-3-ylsulfonyl)piperidine-1-carboxylate

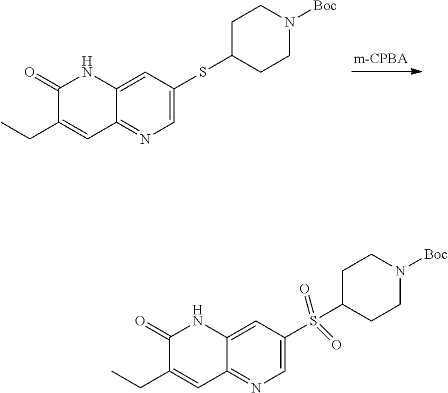 [SMILES: CCc1cc2ncc(SC3CCN(C(=O)OC(C)(C)C)CC3)cc2[nH]c1=O ; CCc1cc2ncc(S(=O)(=O)C3CCN(C(=O)OC(C)(C)C)CC3)cc2[nH]c1=O]

To a mixture of tert-butyl 4-[(7-ethyl-6-oxo-5H-1,5-naphthyridin-3-yl)sulfanyl]piperidine-1-carboxylate (600.0 mg, 1.54 mmol) in CHCl₃ (15.0 mL) was added m-CPBA (531.6 mg, 3.08 mmol) at room temperature. The resulting mixture was stirred at room temperature for 2 h. After the reaction was completed, the resulting mixture was concentrated under reduced pressure. The residue was purified by flash column chromatography with CH₂Cl₂/MeOH (10/1, v/v) to afford tert-butyl 4-(7-ethyl-6-oxo-5H-1,5-naphthyridin-3-ylsulfonyl)piperidine-1-carboxylate (350.0 mg, 53%) as a white solid. LCMS (ESI, m/z): [M+H]⁺=422.2.

### Step 7. Synthesis of 3-ethyl-7-(piperidine-4-sulfonyl)-1H-1,5-naphthyridin-2-one

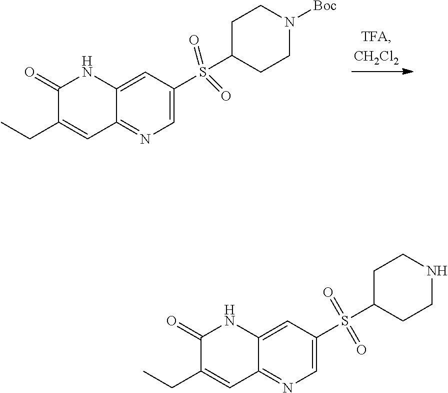 [SMILES: CCc1cc2ncc(S(=O)(=O)C3CCN(C(=O)OC(C)(C)C)CC3)cc2[nH]c1=O ; CCc1cc2ncc(S(=O)(=O)C3CCNCC3)cc2[nH]c1=O]

To a mixture of tert-butyl 4-(7-ethyl-6-oxo-5H-1,5-naphthyridin-3-ylsulfonyl)piperidine-1-carboxylate (350.0 mg, 0.83 mmol) in CH₂Cl₂ (5.0 mL) was added TFA (2.5 mL) at room temperature. The resulting mixture was stirred at room temperature for 1 h. After the reaction was completed, the mixture was basified to pH=7 with saturated NaHCO₃ (aq.). The resulting mixture was extracted with CH₂Cl₂. The combined organic layers were washed with brine, dried over anhydrous sodium sulfate and filtered. The filtrate was concentrated under reduced pressure. The residue was purified by reverse phase flash column chromatography with CH₃CN/H₂O (1/1, v/v) to afford 3-ethyl-7-(piperidine-4-sulfonyl)-1H-1,5-naphthyridin-2-one (120.0 mg, 44%) as a white solid. LCMS (ESI, m/z): [M+H]⁺=322.1.

### Step 8. Synthesis of Methyl 5-[4-(7-ethyl-6-oxo-5H-1,5-naphthyridin-3-ylsulfonyl)piperidin-1-yl]pyridine-2-carboxylate

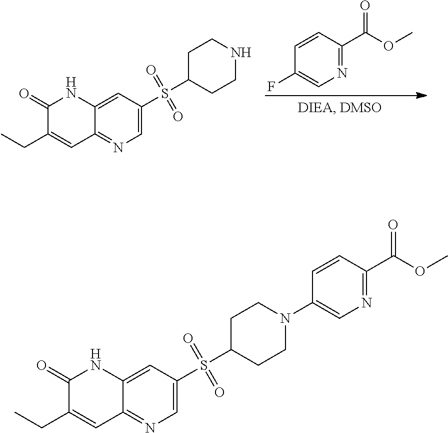

To a mixture of 3-ethyl-7-(piperidine-4-sulfonyl)-1H-1,5-naphthyridin-2-one (120.0 mg, 0.37 mmol) in DMSO (5.0 mL) was added methyl 5-fluoropyridine-2-carboxylate (115.8 mg, 0.74 mmol) and DIEA (241.2 mg, 1.86 mmol) at room temperature. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford methyl 5-[4-(7-ethyl-6-oxo-5H-1,5-naphthyridin-3-ylsulfonyl)piperidin-1-yl]pyridine-2-carboxylate (70.0 mg, 41%) as a white solid. LCMS (ESI, m/z): [M+H]⁺=457.1.

### Step 9. Synthesis of 5-[4-(7-ethyl-6-oxo-5H-1,5-naphthyridin-3-ylsulfonyl)piperidin-1-yl]-N-methylpyridine-2-carboxamide (Compound 2.1)

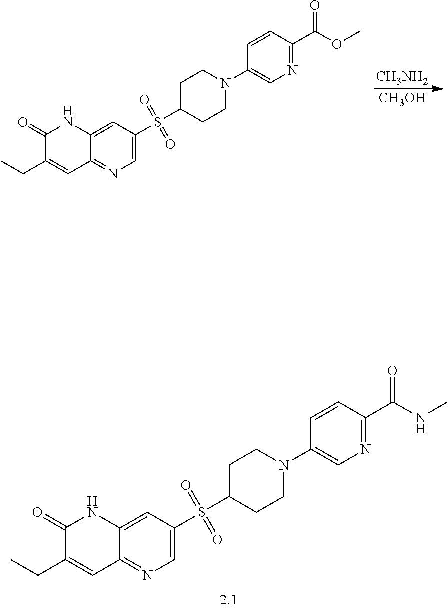

A mixture of methyl 5-[4-(7-ethyl-6-oxo-5H-1,5-naphthyridin-3-ylsulfonyl)piperidin-1-yl]pyridine-2-carboxylate (65.0 mg, 0.14 mmol) in methylamine/methanol (2.0 mL, 2 mol/L) was stirred at 70° C. for 2 h. After the reaction was completed, the resulting mixture was concentrated under reduced pressure. The residue was purified by Prep-HPLC with the following conditions: (Column: XSelect CSH Prep C18 OBD Column, 19×250 mm, 5 m; Mobile Phase A: Water (10 mmol/L NH₄HCO₃), Mobile Phase B: ACN; Flow rate: 20 mL/min; Gradient: 22% B to 27% B in 10 min; Wave Length: 254 nm) to afford 5-[4-(7-ethyl-6-oxo-5H-1,5-naphthyridin-3-ylsulfonyl)piperidin-1-yl]-N-methylpyridine-2-carboxamide (Compound 2.1, 35.7 mg, 55%) as a light yellow solid. LCMS (ESI, m/z): [M+H]⁺=456.2. ¹H NMR (400 MHz, DMSO-d₆): δ 12.18 (s, 1H), 8.79 (d, J=2.0 Hz, 1H), 8.41-8.37 (m, 1H), 8.26 (d, J=2.8 Hz, 1H), 8.06 (d, J=1.6 Hz, 1H), 7.89 (s, 1H), 7.87-7.80 (m, 1H), 7.41-7.38 (m, 1H), 4.11-4.02 (m, 2H), 3.80-3.71 (m, 1H), 2.95-2.84 (m, 2H), 2.78 (d, J=4.8 Hz, 3H), 2.63-2.58 (m, 2H), 1.97-1.93 (m, 2H), 1.70-1.60 (m, 2H), 1.22-1.19 (m, 3H).

### Example 2. Synthesis of 5-(4-((7-ethyl-6-oxo-5,6-dihydro-1,5-naphthyridin-3-yl)sulfonyl)piperazin-1-yl)-N-methylpicolinamide (Compound 2.2)

### Step 1. Synthesis of 7-(benzylthio)-3-ethyl-1,5-naphthyridin-2(1H)-one

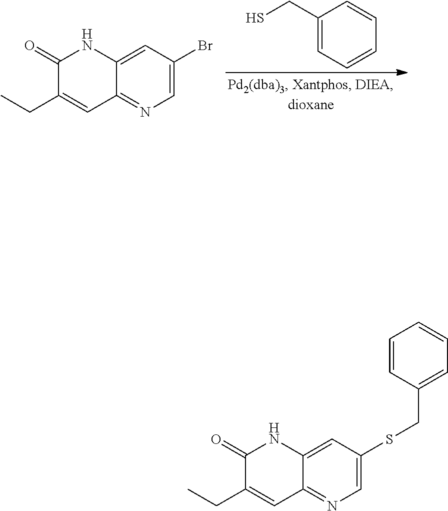 [SMILES: CCc1cc2ncc(Br)cc2[nH]c1=O ; SCc1ccccc1 ; CCc1cc2ncc(SCc3ccccc3)cc2[nH]c1=O]

To a mixture of 7-bromo-3-ethyl-1,5-naphthyridin-2(1H)-one (500.0 mg, 1.98 mmol) and phenylmethanethiol (204.4 mg, 1.65 mmol) in 1,4-dioxane (5.0 mL) were added DIEA (425.5 mg, 3.29 mmol), XantPhos (95.2 mg, 0.16 mmol) and Pd₂(dba)₃ (75.3 mg, 0.08 mmol) at room temperature under N₂. The resulting mixture was stirred at 100° C. for 16 h under N₂. After the reaction was completed, the mixture was diluted with H₂O and extracted with ethyl acetate. The combined organic layers were washed with brine, dried over anhydrous sodium sulfate and filtered. The filtrate was concentrated under reduced pressure. The residue was purified by flash column chromatography with petroleum ether/ethyl acetate (5/1, v/v) to afford 7-(benzylthio)-3-ethyl-1,5-naphthyridin-2(1H)-one (210.0 mg, 43%) as a light yellow solid. LCMS (ESI, m/z): [M+H]⁺=297.1.

### Step 2. Synthesis of 7-ethyl-6-oxo-5,6-dihydro-1,5-naphthyridine-3-sulfonyl chloride

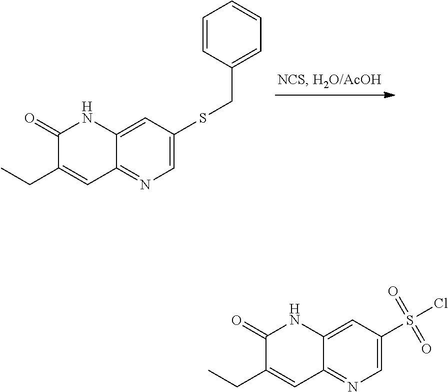 [SMILES: CCc1cc2ncc(SCc3ccccc3)cc2[nH]c1=O ; CCc1cc2ncc(S(=O)(=O)Cl)cc2[nH]c1=O]

To a solution of 7-(benzylthio)-3-ethyl-1,5-naphthyridin-2(1H)-one (210.0 mg, 0.79 mmol) in H₂O (2.0 mL)/AcOH (6.0 mL) were added NCS (397.2 mg, 2.97 mmol) at room temperature. The resulting mixture was stirred at room temperature for 2 h. After the reaction was completed, the mixture was diluted with H₂O and extracted with ethyl acetate. The combined organic layers were washed with brine, dried over anhydrous sodium sulfate and filtered. The filtrate was concentrated under reduced pressure to afford 7-ethyl-6-oxo-5,6-dihydro-1,5-naphthyridine-3-sulfonyl chloride (200 mg, crude) as a light yellow solid. LCMS (ESI, m/z): [M+H]⁺=273.0.

### Step 3. Synthesis of 5-(4-((7-ethyl-6-oxo-5,6-dihydro-1,5-naphthyridin-3-yl)sulfonyl)piperazin-1-yl)-N-methylpicolinamide (Compound 2.2)

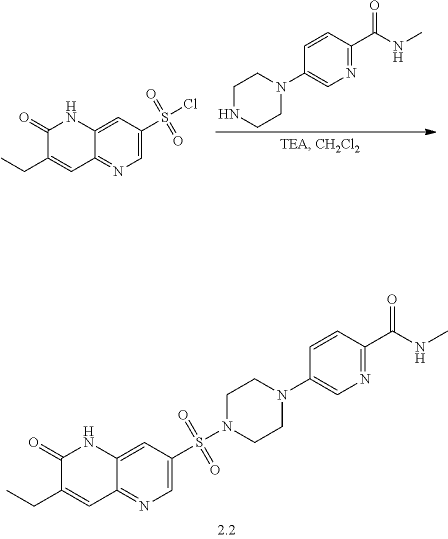 [SMILES: CCc1cc2ncc(S(=O)(=O)Cl)cc2[nH]c1=O ; CNC(=O)c1ccc(N2CCNCC2)cn1 ; CCc1cc2ncc(S(=O)(=O)N3CCN(c4ccc(C(=O)NC)nc4)CC3)cc2[nH]c1=O]

To a solution of N-methyl-5-(piperazin-1-yl)picolinamide (96.9 mg, 0.44 mmol) in CH₂Cl₂ (8.0 mL) were added 7-ethyl-6-oxo-5,6-dihydro-1,5-naphthyridine-3-sulfonyl chloride (180.0 mg, crude) and TEA (45.9 mg, 0.66 mmol) at room temperature. The resulting mixture was stirred at room temperature for 2 h. After the reaction was completed, the resulting mixture was concentrated under reduced pressure. The residue was purified by flash column chromatography with CH₂Cl₂/MeOH (10/1, v/v) to afford 5-(4-((7-ethyl-6-oxo-5,6-dihydro-1,5-naphthyridin-3-yl)sulfonyl)piperazin-1-yl)-N-methylpicolinamide (Compound 2.2, 30.2 mg, 15%) as an off white solid. LCMS (ESI, m/z): [M+H]⁺=457.1. ¹H NMR (400 MHz, DMSO-d₆): δ 12.12 (s, 1H), 8.73 (d, J=2.0 Hz, 1H), 8.41-8.39 (m, 1H), 8.23 (d, J=2.8 Hz, 1H), 7.99 (d, J=1.6 Hz, 1H), 7.86 (s, 1H), 7.81 (d, J=8.8 Hz, 1H), 7.39-7.36 (m, 1H), 3.47-3.44 (m, 4H), 3.16-3.14 (m, 4H), 2.76 (d, J=4.8 Hz, 3H), 2.60-2.57 (m, 2H), 1.21-1.17 (m, 3H).

### Example 3. Synthesis of 5-[4-(2-ethyl-3-oxo-4H-quinoxalin-6-ylsulfonyl)piperidin-1-yl]-N-methylpyridine-2-carboxamide (Compound 2.3)

### Step 1. Synthesis of 7-bromo-3-ethyl-1H-quinoxalin-2-one

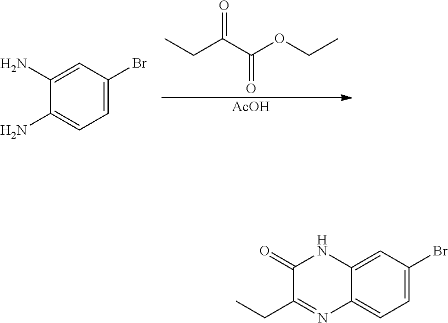

To a solution of 4-bromobenzene-1,2-diamine (10.0 g, 53.46 mmol) in AcOH (100.0 mL) was added ethyl 2-oxobutanoate (7.0 g, 53.46 mmol) at room temperature. The resulting mixture was stirred at room temperature for 0.5 h. After the reaction was completed, the reaction mixture was neutralized to pH=7 with saturated NaHCO₃ (aq.) at 0° C. The resulting mixture was diluted with H₂O and extracted with EtOAc. The combined organic layers were washed with brine and dried over anhydrous Na₂SO₄. After filtration, the filtrate was concentrated under reduced pressure. The residue was purified by flash column chromatography with petroleum ether/ethyl acetate (1/9, v/v) to afford 7-bromo-3-ethyl-1H-quinoxalin-2-one (1.5 g, 11%) as a yellow solid. LCMS (ESI, m/z): [M+H]⁺=253.1.

### Step 2. Synthesis of tert-butyl 4-[(2-ethyl-3-oxo-4H-quinoxalin-6-yl)sulfanyl]piperidine-1-carboxylate

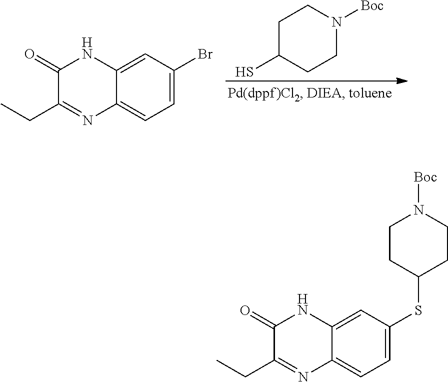 [SMILES: CCc1nc2ccc(Br)cc2[nH]c1=O ; CC(C)(C)OC(=O)N1CCC(S)CC1 ; CCc1nc2ccc(SC3CCN(C(=O)OC(C)(C)C)CC3)cc2[nH]c1=O]

To a mixture of 7-bromo-3-ethyl-1H-quinoxalin-2-one (700.0 mg, 2.77 mmol) and tert-butyl 4-sulfanylpiperidine-1-carboxylate (601.1 mg, 2.77 mmol) in toluene (10.0 mL) was added DIEA (714.9 mg, 5.53 mmol) and Pd(dppf)Cl₂ (404.7 mg, 0.55 mmol) at room temperature under N₂. The resulting mixture was stirred at 110° C. for 2 h under N₂. After the reaction was completed, the reaction mixture was concentrated under reduced pressure. The residue was purified by flash column chromatography with petroleum ether/ethyl acetate (1/1, v/v) to afford tert-butyl 4-[(2-ethyl-3-oxo-4H-quinoxalin-6-yl)sulfanyl]piperidine-1-carboxylate (500.0 mg, 46%) as a light yellow solid. LCMS (ESI, m/z): [M+H]⁺=390.1.

### Step 3. Synthesis of tert-butyl 4-(2-ethyl-3-oxo-4H-quinoxalin-6-ylsulfonyl)piperidine-1-carboxylate

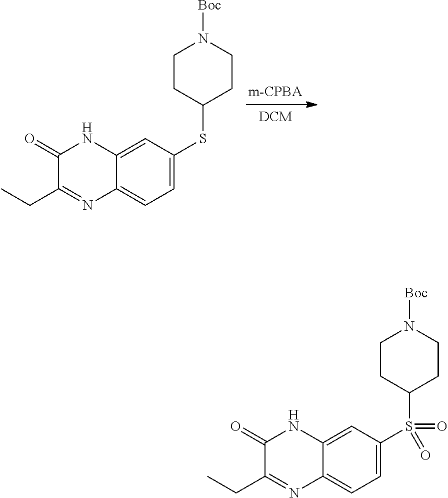 [SMILES: CCc1nc2ccc(SC3CCN(C(=O)OC(C)(C)C)CC3)cc2[nH]c1=O ; CCc1nc2ccc(S(=O)(=O)C3CCN(C(=O)OC(C)(C)C)CC3)cc2[nH]c1=O]

To a mixture of tert-butyl 4-[(2-ethyl-3-oxo-4H-quinoxalin-6-yl)sulfanyl]piperidine-1-carboxylate (500.0 mg, 1.28 mmol) in DCM (5.0 mL) was added m-CPBA (332.3 mg, 1.93 mmol) at room temperature. The resulting mixture was stirred at room temperature for 2 h. To the above mixture was added a new batch of m-CPBA (221.5 mg, 1.28 mmol) at room temperature. The resulting mixture was stirred at room temperature for additional 1 h. After the reaction was completed, the reaction mixture was diluted with water and extracted with DCM. The combined organic layers were washed with brine, dried over anhydrous Na₂SO₄ and filtered. The filtrate was concentrated under reduced pressure. The residue was purified by flash column chromatography with petroleum ether/ethyl acetate (1/1, v/v) to afford tert-butyl 4-(2-ethyl-3-oxo-4H-quinoxalin-6-ylsulfonyl)piperidine-1-carboxylate (300.0 mg, 55%) as a yellow solid. LCMS (ESI, m/z): [M+H]⁺=422.2.

### Step 4. Synthesis of 3-ethyl-7-(piperidine-4-sulfonyl)-1H-quinoxalin-2-one

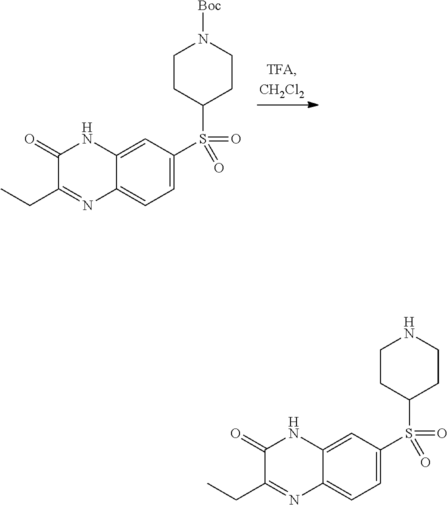 [SMILES: CCc1nc2ccc(S(=O)(=O)C3CCN(C(=O)OC(C)(C)C)CC3)cc2[nH]c1=O ; CCc1nc2ccc(S(=O)(=O)C3CCNCC3)cc2[nH]c1=O]

To a mixture of tert-butyl 4-(2-ethyl-3-oxo-4H-quinoxalin-6-ylsulfonyl)piperidine-1-carboxylate (300.0 mg, 0.71 mmol) in DCM (4.0 mL) was added TFA (2.0 mL) at room temperature. The resulting mixture was stirred at room temperature for 0.5 h. After the reaction was completed, the mixture was neutralized to pH=7 with saturated NaHCO₃ (aq.). The resulting mixture was extracted with CH₂Cl₂. The combined organic layers were washed with brine, dried over anhydrous Na₂SO₄ and filtered. The filtrate was concentrated under reduced pressure. The residue was purified by reverse phase flash column chromatography with ACN/H₂O (50/50, v/v) to afford 3-ethyl-7-(piperidine-4-sulfonyl)-1H-quinoxalin-2-one (80.0 mg, 34%) as a light yellow solid. LCMS (ESI, m/z): [M+H]⁺=322.2.

### Step 5. Synthesis of methyl 5-[4-(2-ethyl-3-oxo-4H-quinoxalin-6-ylsulfonyl)piperidin-1-yl]pyridine-2-carboxylate

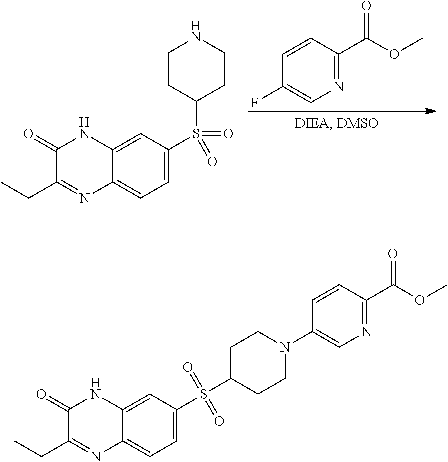

To a mixture of 3-ethyl-7-(piperidine-4-sulfonyl)-1H-quinoxalin-2-one (80.0 mg, 0.25 mmol) and methyl 5-fluoropyridine-2-carboxylate (38.6 mg, 0.25 mmol) in DMSO (2.0 mL) was added DIEA (96.5 mg, 0.75 mmol) at room temperature. The resulting mixture was stirred at 120° C. for 16 h. After the reaction was completed, the resulting mixture was purified by reverse phase flash column chromatography with ACN/H₂O (60/40, v/v) to afford methyl 5-[4-(2-ethyl-3-oxo-4H-quinoxalin-6-ylsulfonyl)piperidin-1-yl]pyridine-2-carboxylate (50.0 mg, 44%) as a light yellow solid. LCMS (ESI, m/z): [M+H]⁺=457.1.

### Step 6. Synthesis of 5-[4-(2-ethyl-3-oxo-4H-quinoxalin-6-ylsulfonyl)piperidin-1-yl]-N-methylpyridine-2-carboxamide (Compound 2.3)

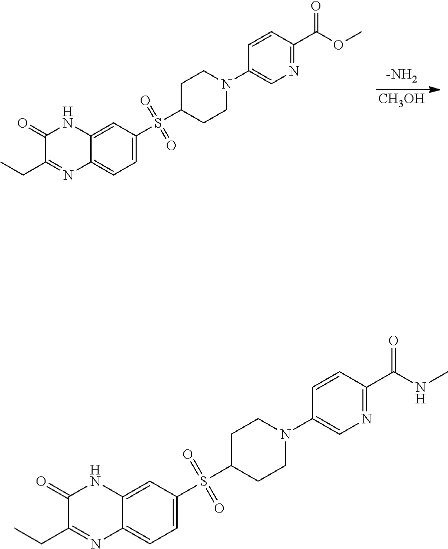

A solution of methyl 5-[4-(2-ethyl-3-oxo-4H-quinoxalin-6-ylsulfonyl)piperidin-1-yl]pyridine-2-carboxylate (50.0 mg, 0.11 mmol) in methanamine (1.0 mL, 33% in MeOH) was stirred at room temperature for 16 h. After the reaction was completed, the mixture was concentrated under reduced pressure. The residue was purified by Prep-HPLC with the following conditions: (Column: XBridge Prep OBD C18 Column, 30×150 mm, 5 m; Mobile Phase A: Water (10 mmol/L NH₄HCO₃), Mobile Phase B: ACN; Flow rate: 6 mL/min; Gradient: 24% B to 32% B in 8 min; Wave Length: 220 nm) to afford 5-[4-(2-ethyl-3-oxo-4H-quinoxalin-6-ylsulfonyl)piperidin-1-yl]-N-methylpyridine-2-carboxamide (Compound 2.3, 21.2 mg, 41%) as a light yellow solid. LCMS (ESI, m/z): [M+H]⁺=456.1. ¹H NMR (400 MHz, DMSO-d₆): δ 12.53 (s, 1H), 8.40-8.36 (m, 1H), 8.26 (d, J=2.8 Hz, 1H), 7.99 (d, J=8.4 Hz, 1H), 7.81 (d, J=8.8 Hz, 1H), 7.76 (d, J=1.6 Hz, 1H), 7.69-7.66 (m, 1H), 7.40-7.37 (m, 1H), 4.04-4.00 (m, 2H), 3.67-3.61 (m, 1H), 2.90-2.83 (m, 4H), 2.78 (d, J=4.8 Hz, 3H), 1.94-1.90 (m, 2H), 1.67-1.57 (m, 2H), 1.26-1.22 (m, 3H).

### Example 4. Synthesis of 5-(4-((2-ethyl-3-oxo-3,4-dihydroquinoxalin-6-yl)sulfonyl)piperazin-1-yl)-N-methylpicolinamide (Compound 2.4)

### Step 1. Synthesis of 7-(benzylthio)-3-ethylquinoxalin-2(1H)-one

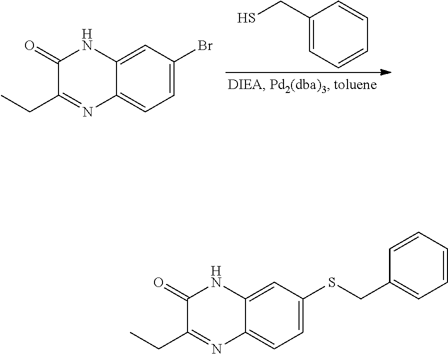 [SMILES: CCc1nc2ccc(Br)cc2[nH]c1=O ; SCc1ccccc1 ; CCc1nc2ccc(SCc3ccccc3)cc2[nH]c1=O]

To a solution of 7-bromo-3-ethylquinoxalin-2(1H)-one (1.0 g, 3.95 mmol) in toluene (15.0 mL) was added phenylmethanethiol (0.9 g, 7.89 mmol), DIEA (1.0 g, 7.90 mmol), XantPhos (0.9 g, 1.58 mmol) and Pd₂(dba)₃ (0.7 g, 0.79 mmol) at room temperature under N₂. The resulting mixture was stirred at 110° C. for 3 h under N₂. After the reaction was completed, the resulting mixture was concentrated under reduced pressure. The residue was purified by flash column chromatography with ethyl acetate/petroleum ether (1/1, v/v) to afford 7-(benzylthio)-3-ethylquinoxalin-2(1H)-one (700.0 mg, 59%) as a yellow solid. LCMS (ESI, m/z): [M+H]⁺=297.1.

### Step 2. Synthesis of 2-(1-chloroethyl)-3-oxo-3,4-dihydroquinoxaline-6-sulfonyl chloride

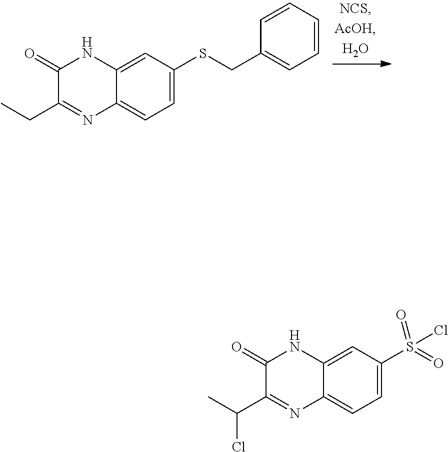 [SMILES: CCc1nc2ccc(SCc3ccccc3)cc2[nH]c1=O ; CC(Cl)c1nc2ccc(S(=O)(=O)Cl)cc2[nH]c1=O]

To a mixture of 7-(benzylthio)-3-ethylquinoxalin-2(1H)-one (200.0 mg, 0.67 mmol) in AcOH (3.0 mL) and H₂O (1.0 mL) was added NCS (360.4 mg, 2.70 mmol) at 0° C. The resulting mixture was stirred at room temper for 2 h. After the reaction was completed, the resulting mixture was concentrated under reduced pressure to afford 2-(1-chloroethyl)-3-oxo-4H-quinoxaline-6-sulfonyl chloride (210.0 mg, crude) as a brown solid. LCMS (ESI, m/z): [M−H]⁻=305.0.

### Step 3. Synthesis of 5-(4-((2-(1-chloroethyl)-3-oxo-3,4-dihydroquinoxalin-6-yl)sulfonyl)piperazin-1-yl)-N-methylpicolinamide

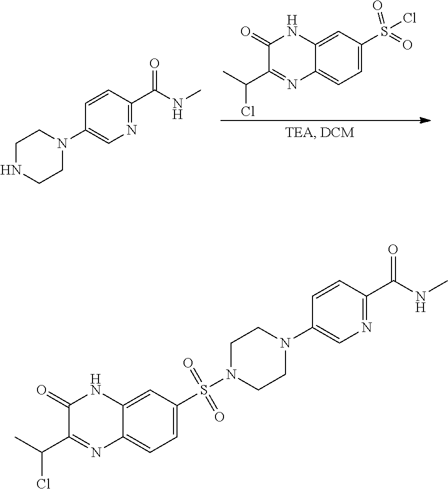

To a mixture of N-methyl-5-(piperazin-1-yl)picolinamide (100.0 mg, 0.45 mmol) in DCM (3.0 mL) was added 2-(1-chloroethyl)-3-oxo-3,4-dihydroquinoxaline-6-sulfonyl chloride (209.1 mg, crude) and TEA (229.7 mg, 2.27 mmol) at room temperature under N₂. The resulting mixture was stirred at room temperature for 2 h under N₂. After the reaction was completed, the resulting mixture was concentrated under reduced pressure. The residue was purified by flash column chromatography with petroleum ether/ethyl acetate (1/1, v/v) to afford 5-{4-[2-(1-chloroethyl)-3-oxo-4H-quinoxalin-6-ylsulfonyl]piperazin-1-yl}-N-methylpyridine-2-carboxamide (89.0 mg, 40%) as a light yellow solid. LCMS (ESI, m/z): [M+H]⁺=491.1.

### Step 4. Synthesis of 5-(4-((2-ethyl-3-oxo-1,2,3,4-tetrahydroquinoxalin-6-yl)sulfonyl)piperazin-1-yl)-N-methylpicolinamide

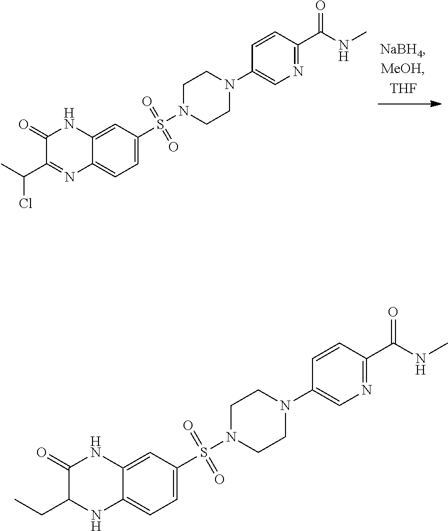

To a mixture of 5-(4-((2-(1-chloroethyl)-3-oxo-3,4-dihydroquinoxalin-6-yl)sulfonyl)piperazin-1-yl)-N-methylpicolinamide (80.0 mg, 0.16 mmol) in THF (3.0 mL) and MeOH (3.0 mL) were added NaBH₄ (15.4 mg, 0.40 mmol) at 0° C. under N₂. The resulting mixture was stirred at room temperature for 2 h under N₂. After the reaction was completed, the reaction mixture was quenched with H₂O at 0° C. and then extracted with ethyl acetate. The combined organic layer was washed with brine, dried over anhydrous sodium sulfate and filtered. The filtrate was concentrated under reduced pressure to afford 5-(4-((2-ethyl-3-oxo-1,2,3,4-tetrahydroquinoxalin-6-yl)sulfonyl)piperazin-1-yl)-N-methylpicolinamide (90.0 mg, crude) as a white solid. LCMS (ESI, m/z): [M+H]⁺=459.2.

### Step 5. Synthesis of 5-(4-((2-ethyl-3-oxo-3,4-dihydroquinoxalin-6-yl)sulfonyl)piperazin-1-yl)-N-methylpicolinamide (Compound 2.4)

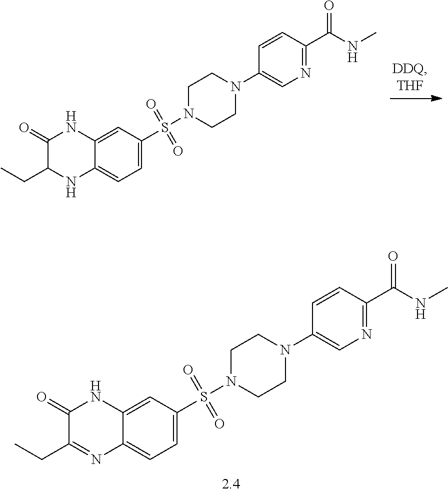

To a mixture of 5-(4-((2-ethyl-3-oxo-1,2,3,4-tetrahydroquinoxalin-6-yl)sulfonyl)piperazin-1-yl)-N-methylpicolinamide (80.0 mg, crude) in THF (3.0 mL) was added DDQ (39.6 mg, 0.17 mmol) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: (Column: XSelect CSH Prep C18 OBD Column, 19×250 mm, 5 m; Mobile Phase A: Water (10 mmol/L NH₄HCO₃), Mobile Phase B: ACN; Flow rate: 25 mL/min; Gradient: 30% B to 40% B in 10 min; Wave Length: 254 nm) to afford 5-[4-(2-ethyl-3-oxo-4H-quinoxalin-6-ylsulfonyl)piperazin-1-yl]-N-methylpyridine-2-carboxamide (Compound 2.4, 11.9 mg, 14%) as a white solid. LCMS (ESI, m/z): [M+H]⁺=457.2. ¹H NMR (400 MHz, DMSO-d₆): δ 12.49 (s, 1H), 8.41-8.37 (m, 1H), 8.23 (d, J=2.8 Hz, 1H), 7.96 (d, J=8.4 Hz, 1H), 7.81 (d, J=8.8 Hz, 1H), 7.68 (d, J=2.0 Hz, 1H), 7.60-7.57 (m, 1H), 7.39-7.36 (m, 1H), 3.46-3.43 (m, 4H), 3.11-3.08 (m, 4H), 2.87-2.82 (m, 2H), 2.76 (d, J=4.8 Hz, 3H), 1.24-1.20 (m, 3H).

### Example 5. Synthesis of 7-((1-(2,4-difluorophenyl)piperidin-4-yl)sulfonyl)-3-ethyl-1,5-naphthyridin-2(1H)-one (Compound 2.5)

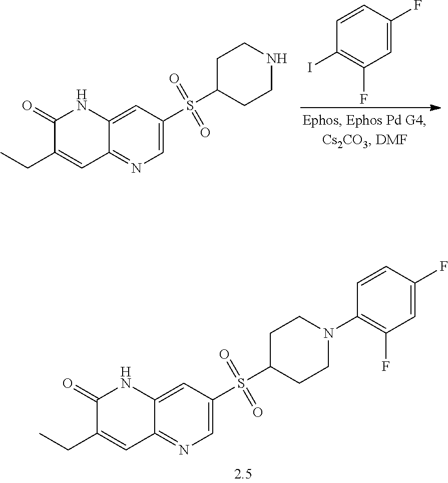

To a solution of 3-ethyl-7-(piperidin-4-ylsulfonyl)-1,5-naphthyridin-2(1H)-one (290.0 mg, 0.90 mmol) in DMF (10.0 mL) were added 2,4-difluoro-1-iodobenzene (148.0 mg, 0.76 mmol), EPhos (193.0 mg, 0.36 mmol), EPhos Pd G4 (165.7 mg, 0.18 mmol) and Cs₂CO₃ (881.9 mg, 2.70 mmol) at room temperature under N₂. The resulting mixture was stirred at 120° C. for 16 h under N₂. After the reaction was completed, the resulting mixture was cooled to room temperature and diluted with H₂O. The mixture was extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (74/26, v/v) and then purified by Prep-HPLC with the following conditions (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 39% B to 49% B in 10 min; Wave Length: 220 nm) to afford 7-((1-(2,4-difluorophenyl)piperidin-4-yl)sulfonyl)-3-ethyl-1,5-naphthyridin-2(1H)-one (Compound 2.5, 20.6 mg, 5%) as a white solid. LCMS (ESI, m/z): [M+H]⁺=434.1. ¹H NMR (400 MHz, DMSO-d₆): δ 12.13 (s, 1H), 8.81 (s, 1H), 8.09 (s, 1H), 7.89 (s, 1H), 7.20-6.95 (m, 3H), 3.62-3.56 (m, 1H), 3.35-3.31 (m, 2H), 2.76-2.58 (m, 4H), 2.08-1.98 (m, 2H), 1.77-1.67 (m, 2H), 1.23-1.19 (m 3H).

### Example 6. Synthesis of 7-((4-(2,4-difluorophenyl)piperazin-1-yl)sulfonyl)-3-ethyl-1,5-naphthyridin-2(1H)-one (Compound 2.6)

### Step 1. Synthesis of tert-butyl 4-(2,4-difluorophenyl)piperazine-1-carboxylate

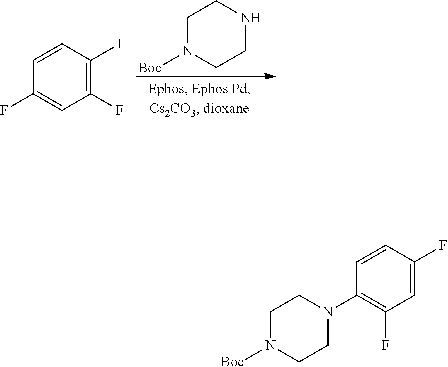

To a mixture of 2,4-difluoro-1-iodobenzene (700.0 mg, 2.91 mmol) in dioxane (10.0 mL) was added tert-butyl piperazine-1-carboxylate (814.9 mg, 4.37 mmol), K₂CO₃ (1209.3 mg, 8.75 mmol), EPhos (623.9 mg, 1.16 mmol) and EPhos Pd G4 (535.8 mg, 0.58 mmol) at room temperature under N₂. The resulting mixture was stirred at 100° C. for 12 h under N₂. After the reaction was completed, the resulting mixture was concentrated under reduced pressure. The residue was purified by flash column chromatography with petroleum ether/ethyl acetate (3/1, v/v) to afford tert-butyl 4-(2,4-difluorophenyl)piperazine-1-carboxylate (600.0 mg, 68%) as a white solid. LCMS (ESI, m/z): [M+H]⁺=299.1.

### Step 2. Synthesis of 1-(2,4-difluorophenyl)piperazine

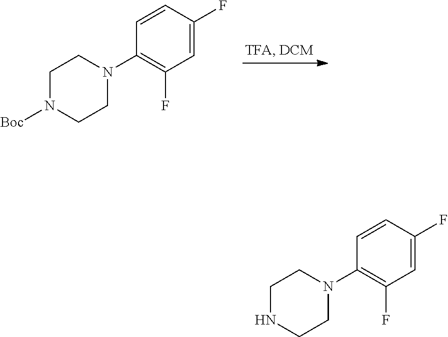 [SMILES: CC(C)(C)OC(=O)N1CCN(c2ccc(F)cc2F)CC1 ; Fc1ccc(N2CCNCC2)c(F)c1]

To a mixture of tert-butyl 4-(2,4-difluorophenyl)piperazine-1-carboxylate (600.0 mg, 1.54 mmol) in DCM (6.0 mL) was added TFA (3.0 mL) at room temperature. The resulting mixture was stirred at room temperature for 30 min. After the reaction was completed, the mixture was basified to pH=8 with saturated NaHCO₃ (aq.). The resulting mixture was extracted with DCM. The combined organic layers were washed with brine, dried over anhydrous sodium sulfate and filtered. The filtrate was concentrated under reduced pressure. The residue was purified by flash column chromatography with CH₂Cl₂/MeOH (10/1, v/v) to afford 1-(2,4-difluorophenyl)piperazine (390.0 mg, 98%) as a white solid. LCMS (ESI, m/z): [M+H]⁺=199.1.

### Step 3. Synthesis of 7-((4-(2,4-difluorophenyl)piperazin-1-yl)sulfonyl)-3-ethyl-1,5-naphthpyridin-2(1H)-one (Compound 2.6)

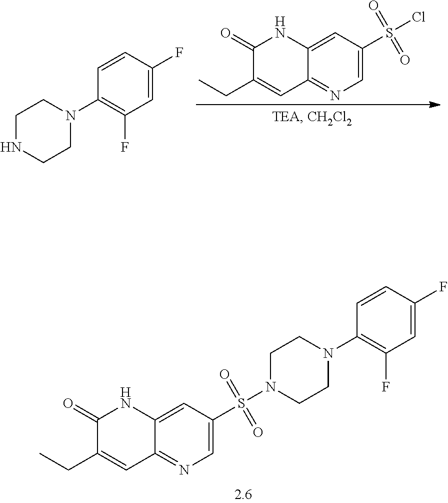

To a solution of 1-(2,4-difluorophenyl)piperazine (200.0 mg, 1.00 mmol) in DCM (7.0 mL) was added TEA (510.5 mg, 5.04 mmol) and 7-ethyl-6-oxo-5,6-dihydro-1,5-naphthyridine-3-sulfonyl chloride (412.7 mg, crude) at 0° C. under N₂. The resulting mixture was stirred at room temperature for 2 h. After the reaction was completed, the resulting mixture was concentrated under reduced pressure. The residue was purified by Prep-HPLC with the following conditions: (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 43% B to 53% B in 10 min; Wave Length: 220 nm) to afford 7-((4-(2,4-difluorophenyl)piperazin-1-yl)sulfonyl)-3-ethyl-1,5-naphthyridin-2(1H)-one (Compound 2.6, 15.4 mg, 3%) as a white solid. LCMS (ESI, m/z): [M+H]⁺=435.1. ¹H NMR (400 MHz, DMSO-d₆): δ 12.00 (s, 1H), 8.73 (s, 1H), 8.00 (s, 1H), 7.88 (s, 1H), 7.19-7.00 (m, 3H), 3.15-3.01 (m, 8H), 2.61-2.57 (m, 2H), 1.24-1.18 (m, 3H).

### Example 7. Synthesis of (R)-5-(4-(7-ethyl-6-oxo-5,6-dihydro-1,5-naphthyridine-3-sulfonimidoyl)piperidin-1-yl)-N-methylpicolinamide (Compound 2.7) and (S)-5-(4-(7-ethyl-6-oxo-5,6-dihydro-1,5-naphthyridine-3-sulfonimidoyl)piperidin-1-yl)-N-methylpicolinamide (Compound 2.8)

### Step 1. Synthesis of 3-ethyl-7-(piperidin-4-ylsulfanyl)-1H-1,5-naphthyridin-2-one

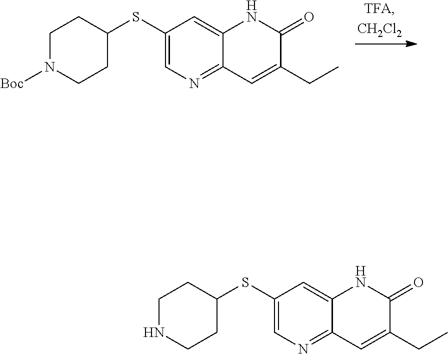 [SMILES: CCc1cc2ncc(SC3CCN(C(=O)OC(C)(C)C)CC3)cc2[nH]c1=O ; CCc1cc2ncc(SC3CCNCC3)cc2[nH]c1=O]

To a solution of tert-butyl 4-[(7-ethyl-6-oxo-5H-1,5-naphthyridin-3-yl)sulfanyl]piperidine-1-carboxylate (1.4 g, 3.59 mmol) in DCM (10.0 mL) was added TFA (3.0 mL) at room temperature. The resulting mixture was stirred at room temperature for 30 min. After the reaction was completed, the resulting mixture was concentrated under reduced pressure. The pH value of the residue was adjusted to 8.0 with saturated NaHCO₃ (aq.). The mixture was extracted with CH₂Cl₂. The combined organic layer was washed with brine, dried over anhydrous sodium sulfate and filtered. The filtrate was concentrated under vacuum to afford 3-ethyl-7-(piperidin-4-ylsulfanyl)-1H-1,5-naphthyridin-2-one (1.2 g, crude) as a yellow oil. LCMS (ESI, m/z): [M+H]⁺=290.1.

### Step 2. Synthesis of methyl 5-{4-[(7-ethyl-6-oxo-5H-1,5-naphthyridin-3-yl)sulfanyl]piperidin-1-yl}pyridine-2-carboxylate

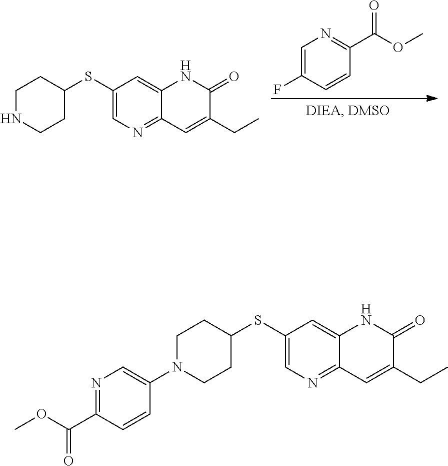

To a solution of 3-ethyl-7-(piperidin-4-ylsulfanyl)-1H-1,5-naphthyridin-2-one (200.0 mg, crude) in DMSO (5.0 mL) was added methyl 5-fluoropyridine-2-carboxylate (160.0 mg, 1.03 mmol) and DIEA (270.0 mg, 2.09 mmol) at room temperature. The resulting mixture was stirred at 100° C. for 16 h. After the reaction was completed, the resulting mixture was cooled to room temperature and diluted with H₂O. The mixture was extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (50/50, v/v) to afford methyl 5-{4-[(7-ethyl-6-oxo-5H-1,5-naphthyridin-3-yl)sulfanyl]piperidin-1-yl}pyridine-2-carboxylate (230.0 mg, 78%) as a yellow solid. LCMS (ESI, m/z): [M+H]⁺=425.1.

### Step 3. Synthesis of 5-{4-[(7-ethyl-6-oxo-5H-1,5-naphthyridin-3-yl)sulfanyl]piperidin-1-yl}-N-methylpyridine-2-carboxamide

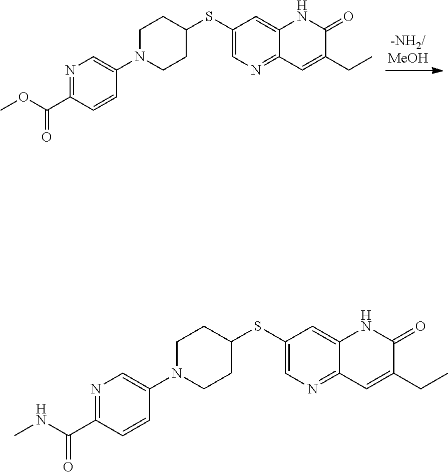

A solution of methyl 5-{4-[(7-ethyl-6-oxo-5H-1,5-naphthyridin-3-yl)sulfanyl]piperidin-1-yl}pyridine-2-carboxylate (500.0 mg, 1.18 mmol) in methylamine/methanol (40.0 mL, 2.0 mol/L) was stirred at 70° C. for 2 h. After the reaction was completed, the resulting mixture was concentrated under reduced pressure. The residue was purified by flash column chromatography with CH₂Cl₂/MeOH (90/10, v/v) to afford 5-{4-[(7-ethyl-6-oxo-5H-1,5-naphthyridin-3-yl)sulfanyl]piperidin-1-yl}-N-methylpyridine-2-carboxamide (170.0 mg, 34%) as a yellow solid. LCMS (ESI, m/z): [M+H]⁺=424.2.

### Step 4. Synthesis of 5-(4-(7-ethyl-6-oxo-5,6-dihydro-1,5-naphthyridine-3-sulfonimidoyl)piperidin-1-yl)-N-methylpicolinamide

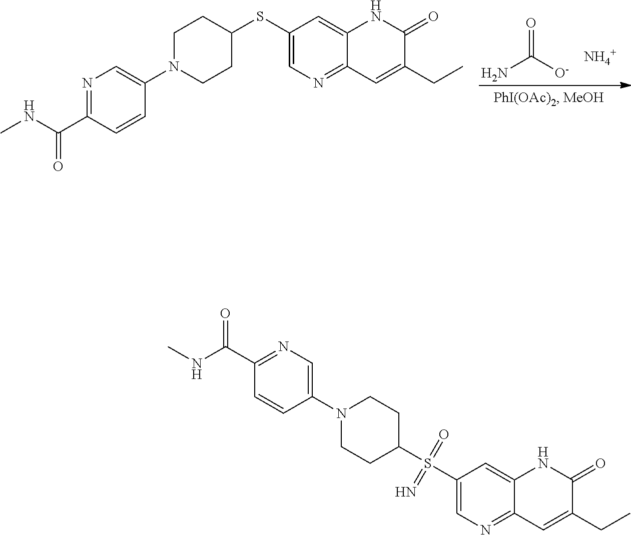

To a solution of 5-{4-[(7-ethyl-6-oxo-5H-1,5-naphthyridin-3-yl)sulfanyl]piperidin-1-yl}-N-methylpyridine-2-carboxamide (200.0 mg, 0.47 mmol) in MeOH (10.0 mL) was added iodosobenzene acetate (424.2 mg, 1.32 mmol) and ammonium carbamate (210.9 mg, 2.20 mmol) at room temperature. The resulting mixture was stirred at room temperature for 30 min. After the reaction was completed, the resulting mixture was filtered. The filtrate was concentrated under reduced pressure. The residue was purified by flash column chromatography with CH₂Cl₂/MeOH (92/8, v/v) to afford 5-(4-(7-ethyl-6-oxo-5,6-dihydro-1,5-naphthyridine-3-sulfonimidoyl)piperidin-1-yl)-N-methylpicolinamide (100.0 mg, 47%) as a white solid. LCMS (ESI, m/z): [M+H]⁺=455.2.

### Step 5. Chiral Separation of (R)-5-(4-(7-ethyl-6-oxo-5,6-dihydro-1,5-naphthyridine-3-sulfonimidoyl)piperidin-1-yl)-N-methylpicolinamide (Compound 2.7) and (S)-5-(4-(7-ethyl-6-oxo-5,6-dihydro-1,5-naphthyridine-3-sulfonimidoyl)piperidin-1-yl)-N-methylpicolinamide (Compound 2.8)

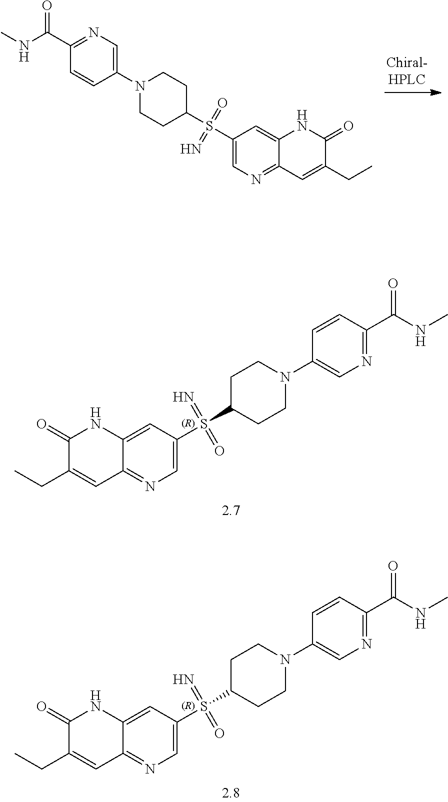

The racemic product of 5-(4-(7-ethyl-6-oxo-5,6-dihydro-1,5-naphthyridine-3-sulfonimidoyl)piperidin-1-yl)-N-methylpicolinamide (100.0 mg, 0.24 mmol) was separated by Prep-Chiral-HPLC with the following conditions: (Column: CHIRAL ART Cellulose-SC, 2×25 cm, 5 m; Mobile Phase A: Hex (0.2% FA)—HPLC, Mobile Phase B: MeOH:DCM=1:1—HPLC; Flow rate: 20 mL/min; Gradient: 80% B to 80% B in 14 min; Wave Length: 254/220 nm; RT1 (min): 10.10; RT2 (min): 13.01) to afford 5-(4-(7-ethyl-6-oxo-5,6-dihydro-1,5-naphthyridine-3-sulfonimidoyl)piperidin-1-yl)-N-methylpicolinamide Isomer A (retention time 10.10 minutes, 27.5 mg, 55%) as a white solid and 5-(4-(7-ethyl-6-oxo-5,6-dihydro-1,5-naphthyridine-3-sulfonimidoyl)piperidin-1-yl)-N-methylpicolinamide Isomer B (retention time 13.01 minutes, 27.5 mg, 55%) as a white solid.

The absolute stereochemistry of Isomers A and B was not assigned. The two isomeric structures that could be obtained from chiral separation of the isomeric mixtures as described above are shown as Compounds 2.7 and 2.8 in Table 1.

5-(4-(7-ethyl-6-oxo-5,6-dihydro-1,5-naphthyridine-3-sulfonimidoyl)piperidin-1-yl)-N-methylpicolinamide Isomer A: RT1 (min): 10.10; LCMS (ESI, m/z): [M+H]⁺=455.1. ¹H NMR (400 MHz, DMSO-d₆): δ 12.15 (s, 1H), 8.77 (d, J=2.0 Hz, 1H), 8.39-8.35 (m, 1H), 8.25 (d, J=2.4 Hz, 1H), 8.10 (d, J=1.6 Hz, 1H), 7.87 (s, 1H), 7.82-7.79 (m, 1H), 7.39-7.36 (m, 1H), 4.76 (s, 1H), 4.07-4.00 (m, 2H), 3.48-3.42 (m, 1H), 2.87-2.77 (m, 5H), 2.63-2.56 (m, 2H), 2.00-1.91 (m, 2H), 1.69-1.54 (m, 2H), 1.22-1.19 (m, 3H).

5-(4-(7-ethyl-6-oxo-5,6-dihydro-1,5-naphthyridine-3-sulfonimidoyl)piperidin-1-yl)-N-methylpicolinamide Isomer B: RT2 (min): 13.01; LCMS (ESI, m/z): [M+H]⁺=455.2. ¹H NMR (400 MHz, DMSO-d₆): δ 12.15 (s, 1H), 8.77 (d, J=1.6 Hz, 1H), 8.37-8.35 (m, 1H), 8.25 (d, J=2.4 Hz, 1H), 8.10 (s, J=1.6 Hz, 1H), 7.87 (s, 1H), 7.82-7.79 (m, 1H), 7.39-7.36 (m, 1H), 4.76 (s, 1H), 4.04-4.01 (m, 2H), 3.49-3.40 (m, 1H), 2.87-2.77 (m, 5H), 2.63-2.56 (m, 2H), 2.01-1.90 (m, 2H), 1.69-1.54 (m, 2H) 1.24-1.19 (m, 3H).

### Biological Examples

### Example B1. PARP1 FP Assay

Compounds were dissolved and 4-fold serially diluted in DMSO. Compounds were transferred by an Echo to the assay plate to reach final concentrations ranging from 10 μM to 0.01 nM.

Assay buffer was composed of 50 mM Tris pH 8.0, 10 mM MgCl₂, 150 mM NaCl and 0.001% Triton X100. PARP1 (BPS Bioscience, Cat #80501) was diluted in the assay buffer to make 40 nM (2×final concentration) enzyme solution. 5 μl of the enzyme solution was then added to each well with compound and the plate was incubated at room temperature for 10 minutes. Probe (TOCRIS, Cat #6461) was diluted in the assay buffer to make 6 nM (2×final concentration) probe solution and 5 μl of the probe solution was added to the wells to start the reaction. The plates were incubated for 4 hours at room temperature.

Fluorescence polarization assays (FP) were performed to measure the activity of kinase. Fluorescent polarization of the PARP1 sample was measured by exciting samples at 480 nm and detecting emission at 535 nm in both parallel and perpendicular channels on Envision (Perkin Elmer). The percentage inhibition was calculated by using the formula: % Inhibition=100×(mP_{HC}−mPₛₐₘₚₗₑ)/(mP_{HC}−mP_{LC}). The low and high control values (‘LC’ and ‘HC’) were generated from wells with only assay buffer or with enzyme and probe treated with 0.1% DMSO, respectively. IC₅₀ values were calculated using XLFit software using a nonlinear regression model with a sigmoidal dose response. The results are shown in Table 2.

TABLE 2

| | Average |
|---|---|
| Cmpd No. | IC₅₀ (nM) |
| 2.1 | 6.18 |
| 2.2 | 27.1 |
| 2.3 | 3.66 |
| 2.4 | 23.9 |
| 2.5 | 3.65 |
| 2.6 | >10.0E+03 |
| 2.7 | 6.89 |
| 2.8 | 8.66 |

### Example B2. PARP2 FP Assay

Compounds were dissolved and 4-fold serially diluted in DMSO. Compounds were transferred by an Echo to the assay plate to reach final concentrations ranging from 10 μM to 0.01 nM.

Assay buffer was composed of 50 mM Tris pH 8.0, 10 mM MgCl₂, 150 mM NaCl and 0.001% Triton X100. PARP2 (BPS Bioscience, Cat #80502) was diluted in the assay buffer to make 40 nM (2×final concentration) enzyme solution. 5 μl of the enzyme solution was then added to each well with compound and the plate was incubated at room temperature for 10 minutes. Probe (TOCRIS, Cat #6461) was diluted in the assay buffer to make 6 nM (2×final concentration) probe solution and 5 μl of the probe solution was added to the wells to start the reaction. The plates were incubated for 4 hours at room temperature.

Fluorescence polarization assays (FP) were performed to measure the activity of kinase. Fluorescent polarization of the PARP2 sample was measured by exciting samples at 480 nm and detecting emission at 535 nm in both parallel and perpendicular channels on Envision (Perkin Elmer). The percentage inhibition was calculated by using the formula: % Inhibition=100×(mP_{HC}−mPₛₐₘₚₗₑ)/(mP_{HC}−mP_{LC}). The low and high control values (‘LC’ and ‘HC’) were generated from wells with only assay buffer or with enzyme and probe treated with 0.1% DMSO, respectively. IC₅₀ values were calculated using XLFit software using a nonlinear regression model with a sigmoidal dose response. The results are shown in Table 3.

TABLE 3

| | Average |
|---|---|
| Cmpd No. | IC₅₀ (nM) |
| 2.1 | >10.0E+03 |
| 2.2 | >10.0E+03 |
| 2.3 | >10.0E+03 |
| 2.4 | >10.0E+03 |
| 2.5 | >10.0E+03 |
| 2.6 | >10.0E+03 |
| 2.7 | >10.0E+03 |
| 2.8 | >10.0E+03 |

### Example B3. PARPylation MSD Assay on MDA-MB-436

MDA-MB-436 cells were grown in the growth medium (DMEM supplemented with 10% FBS and 1× Penicillin-Streptomycin) and harvested at 70-80% confluence. MDA-MB-436 cells were seeded into a 96-well at a density of 1.0*10⁶ cells/100 μl/well. The plates were placed in a 37° C., 5% CO₂ incubator overnight in order to allow cells to adhere.

Compounds were dissolved and 4-fold serially diluted in DMSO. The compounds were then added, mixed, and incubated for 4 hours at 37° C., 5% CO₂. Compounds were added using four-fold dilutions at final concentrations ranging from 1 μM to 0.0038 nM.

Following the four-hour incubation with compounds, cells were washed by 100 μl PBS followed by the removal of PBS. 35 μL of cell lysis buffer (Boston BioProducts, cat #BP-115D) supplemented with 1× protease/phosphatase inhibitor cocktail (Thermo Scientific™, cat #78447) was then added to the washed cells. Cells were incubated with lysis buffer for 20 minutes at 4° C., and then stored at −80° C.

An electrochemiluminescent-based (MSD) assay was perform to detect the PARylation level in MDA-MB-436. A capture antibody able to detect poly- and mono-ADP ribose (CST, Cat #83732) was 1:300 diluted in PBS and then added 40 μL/well to MSD plate and incubated at 4° C. overnight. The next day, plates were washed with 150 μL of 1×TBST (CST, cat #9997S) for 3 times. 150 μL of the 3% Blocker A blocking solution (MSD, cat R⁹³BA-4) was then added for 1 hour at room temperature, with shaking. Plates were washed with 150 μL of 1×TBST for 3 times. Cell lysates were thawed and 35 μL of lysate was added to the MSD plate. The plates were incubated for 1 hour at room temperature, with shaking. MSD plates were then washed with 150 μL of 1×TBST for 3 times followed by addition of 25 μL of the primary detection antibody (1:300 diluted in the Blocker A blocking solution) which was able to detect poly-ADP ribose) (Trevigen, cat 4335-MC-100). Plates were incubated for 1 hour at room temperature, with shaking. Plates were then washed with 150 μL of 1×TBST for 3 times and 25 μL of the secondary detection antibody (1:1000 diluted in the Blocker A blocking solution), which detected mouse antibody (MSD, R32AC-1), was added. Plates were incubated for 1 hour at room temperature, with shaking. Plates were washed with 150 μL of 1×TBST for 3 times. 150 μL of 1×MSD read buffer (MSD, cat R92TC-1) was added to the plates and the plates were read with MSD Sector S600.

The compound inhibition activity was calculated by using formula: % vehicle=100×(ReadoutSample−ReadoutLC)/(ReadoutHC−ReadoutLC). The low and high control values (‘LC’ and ‘HC’) were generated from lysate from wells without cells or with cells treated with 0.1% DMSO, respectively. IC₅₀ values were calculated using XLFit software using a nonlinear regression model with a sigmoidal dose response. The results are shown in Table 4.

TABLE 4

| | Average |
|---|---|
| Cmpd No. | IC₅₀ (nM) |
| 2.1 | 4.97 |
| 2.2 | 180 |
| 2.3 | 9.11 |
| 2.4 | 890 |
| 2.5 | 151 |
| 2.6 | >1.00E+03 |
| 2.7 | 10.8 |
| 2.8 | 0.515 |

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

What is claimed is:

1. A compound of formula (I) 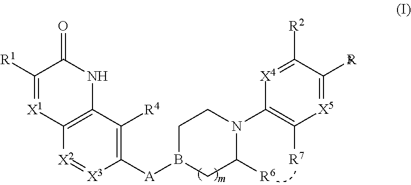
or a pharmaceutically acceptable salt, solvate, hydrate, or co-crystal thereof, or a mixture of any of the foregoing, wherein:
X¹, X² and X³ are each independently N or C—R¹;
X⁴ and X⁵ are each independently N or CH;
A is —S(═O)₂—, —S(═O)(═NH)— or —S(═O)(═N—CH₃)—;
B is N or CH;
each R¹ is independently H, halogen, C₁-C₄ alkyl, C₁-C₄ fluoroalkyl, C₁-C₄ alkoxy, or C₃-C₆ cycloalkyl;
R² is H, halogen, C₁-C₄ alkyl, or C₁-C₄ fluoroalkyl;
R³ is halogen or —C(═O)NH—R⁵;
R⁴ is H, halogen, C₁-C₄ alkyl, or C₁-C₄ fluoroalkyl; and
each R⁵ is independently H, CD₃, C₁-C₄ alkyl, C₁-C₄ fluoroalkyl, C₁₋₄ alkoxy, C₃-C₆ cycloalkyl, or 4- to 7-membered heterocycloalkyl;
R⁶ is H; and
R⁷ is H, halogen, CN, C₁-C₄ alkyl, C₁-C₄ fluoroalkyl, C₁-C₄ alkoxy, or C₃-C₆ cycloalkyl; or
R⁶ and R⁷ are taken together with the atoms to which they are attached to form a ring, wherein R⁶ is —(CH₂)ₙ— and R⁷ is E, wherein E is —O—, —S—, —NR⁵—, or —CR⁵—;
m is an integer of 1 or 2, wherein when R⁶ is H, then m is 1; and
n is an integer of 1 or 2.

2. The compound of claim 1, or a pharmaceutically acceptable salt, solvate, hydrate, or co-crystal thereof, or a mixture of any of the foregoing, wherein the compound is a compound of formula (I-1) 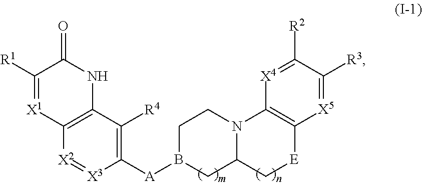
wherein:
E is —O—, —S—, —NR¹—, or —CR⁵—;
m is an integer of 1 or 2; and
n is an integer of 1 or 2.

3. The compound of claim 1, or a pharmaceutically acceptable salt, solvate, hydrate, or co-crystal thereof, or a mixture of any of the foregoing, wherein the compound is a compound of formula (I-2) 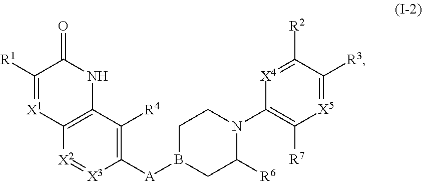
wherein:
R⁶ is H; and
R⁷ is H, halogen, CN, C₁-C₄ alkyl, C₁-C₄ fluoroalkyl, C₁-C₄ alkoxy, or C₃-C₆ cycloalkyl.

4. The compound of claim 1, or a pharmaceutically acceptable salt, solvate, hydrate, or co-crystal thereof, or a mixture of any of the foregoing, wherein A is —S(O)₂—.

5. The compound of claim 1, or a pharmaceutically acceptable salt, solvate, hydrate, or co-crystal thereof, or a mixture of any of the foregoing, wherein A is —S(O)(═NH)—.

6. The compound of claim 1, or a pharmaceutically acceptable salt, solvate, hydrate, or co-crystal thereof, or a mixture of any of the foregoing, wherein A is —S(O)(═N—CH₃)—.

7. The compound of claim 1, or a pharmaceutically acceptable salt, solvate, hydrate, or co-crystal thereof, or a mixture of any of the foregoing, wherein B is N.

8. The compound of claim 1, or a pharmaceutically acceptable salt, solvate, hydrate, or co-crystal thereof, or a mixture of any of the foregoing, wherein B is CH.

9. The compound of claim 1, or a pharmaceutically acceptable salt, solvate, hydrate, or co-crystal thereof, or a mixture of any of the foregoing, wherein the compound is a compound of formula (I-1-a), (I-1-b), (I-1-c), (I-1-d), (I-1-e), or (I-1-f):

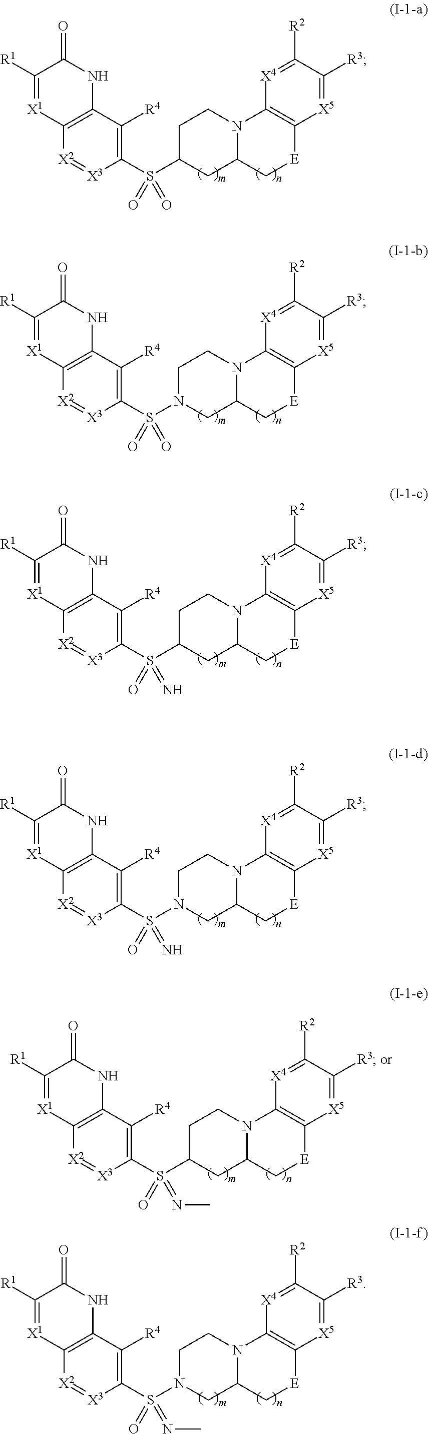

10. The compound of claim 1, or a pharmaceutically acceptable salt, solvate, hydrate, or co-crystal thereof, or a mixture of any of the foregoing, wherein the compound is a compound of formula (I-2-a), (I-2-b), (I-2-c), (I-2-d), (I-2-e), or (I-2-f):

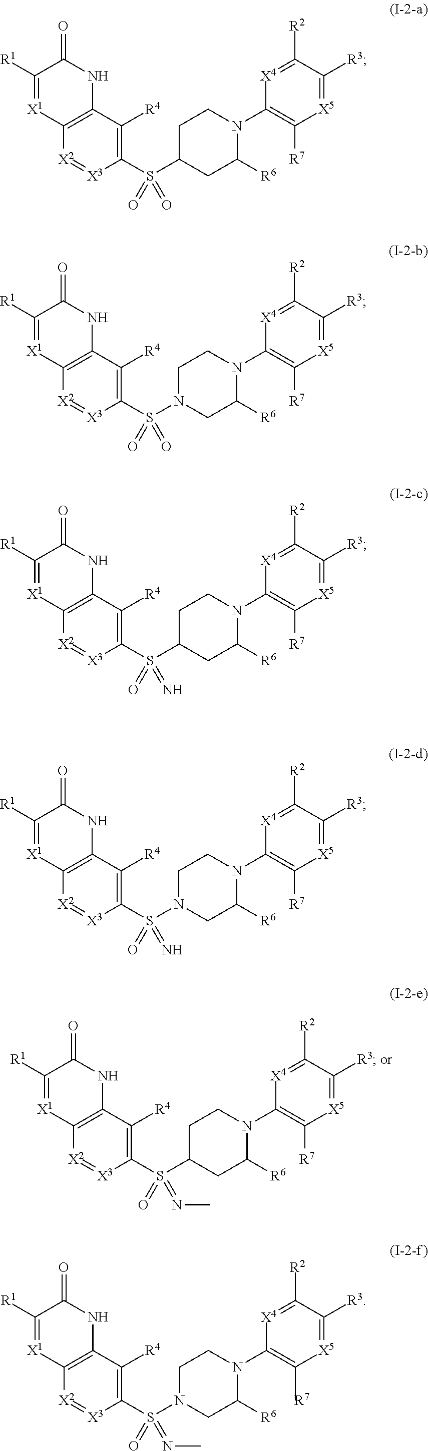

11. The compound of claim 1, or a pharmaceutically acceptable salt, solvate, hydrate, or co-crystal thereof, or a mixture of any of the foregoing, wherein the compound is selected from Table 1.
